(19) United States
(12) Patent Application Publication
(10) Pub. No.: US 2002/0055478 A1
(43) Pub. Date: May 9, 2002

(54) GTP-binding protein useful in treatment and detection of cancer

(76) Inventors: Mary Faris, Los Angeles, CA (US); Pia M. Challita-Eid, Encino, CA (US); Arthur B. Raitano, Los Angeles, CA (US); Steve Chappell Mitchell, Santa Monica, CA (US); Daniel E.H. Afar, Brisbane, CA (US); Aya Jakobovits, Beverly Hills, CA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 09/834,765

(22) Filed: Apr. 12, 2001

Related U.S. Application Data
(60) Provisional application No. 60/196,647, filed on Apr. 12, 2000.

(51) Int. Cl. A61K 48/00; C12Q 1/68; G01N 33/574

(52) U.S. Cl. 514/44; 435/6; 435/7.23

(57) ABSTRACT

A novel gene (designated 103P3E8) and its encoded protein are described. While 103P3E8 exhibits tissue specific expression in normal adult tissue, it is aberrantly expressed in multiple cancers including prostate, bladder, kidney, colon, lung, breast, rectal and stomach cancers. Consequently, 103P3E8 provides a diagnostic and/or therapeutic target for cancers, and the 103P3E8 gene or fragment thereof, or its encoded protein or a fragment thereof can be used to elicit an immune response.

This application claims the benefit of U.S. provisional patent application Ser. No. 60/196,647, filed Apr. 12, 2000, the entire contents of which are incorporated herein by reference.

### FIELD OF THE INVENTION

The invention described herein relates to a novel gene and its encoded protein, termed 103P3E8, and to diagnostic and therapeutic methods and compositions useful in the management of various cancers that express 103P3E8.

### BACKGROUND OF THE INVENTION

Cancer is the second leading cause of human death next to coronary disease. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise. In the early part of the next century, cancer is predicted to become the leading cause of death.

Worldwide, several cancers stand out as the leading killers. In particular, carcinomas of the lung, prostate, breast, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Furthermore, many cancer patients experience a recurrence.

Worldwide, prostate cancer is the fourth most prevalent cancer in men. In North America and Northern Europe, it is by far the most common cancer in males and is the second leading cause of cancer death in men. In the United States alone, well over 30,000 men die annually of this disease—second only to lung cancer. Despite the magnitude of these Figures, there is still no effective treatment for metastatic prostate cancer. Surgical prostatectomy, radiation therapy, hormone ablation therapy, surgical castration and chemotherapy continue to be the main treatment modalities. Unfortunately, these treatments are ineffective for many and are often associated with undesirable consequences.

On the diagnostic front, the lack of a prostate tumor marker that can accurately detect early-stage, localized tumors remains a significant limitation in the diagnosis and management of this disease. Although the serum prostate specific antigen (PSA) assay has been a very useful tool, however its specificity and general utility is widely regarded as lacking in several important respects. Progress in identifying additional specific markers for prostate cancer has been improved by the generation of prostate cancer xenografts that can recapitulate different stages of the disease in mice.

The LAPC (Los Angeles Prostate Cancer) xenografts are prostate cancer xenografts that have survived passage in severe combined immune deficient (SCID) mice and have exhibited the capacity to mimic the transition from androgen dependence to androgen independence (Klein et al., 1997, Nat. Med. 3:402). More recently identified prostate cancer markers include PCTA-1 (Su et al., 1996, Proc. Natl. Acad. Sci. USA 93: 7252), prostate-specific membrane (PSM) antigen (Pinto et al., Clin Cancer Res Sep. 2, 1996 (9): 1445-51), STEAP (Hubert, et al., Proc Natl Acad Sci U S A. Dec. 7, 1999; 96(25): 14523-8) and prostate stem cell antigen (PSCA) (Reiter et al., 1998, Proc. Natl. Acad. Sci. USA 95: 1735).

While previously identified markers such as PSA, PSM, PCTA and PSCA have facilitated efforts to diagnose and treat prostate cancer, there is need for the identification of additional markers and therapeutic targets for prostate and related cancers in order to further improve diagnosis and therapy.

Renal cell carcinoma (RCC) accounts for approximately 3 percent of adult malignancies. Once adenomas reach a diameter of 2 to 3 cm, malignant potential exists. In the adult, the two principal malignant renal tumors are renal cell adenocarcinoma and transitional cell carcinoma of the renal pelvis or ureter. The incidence of renal cell adenocarcinoma is estimated at more than 29,000 cases in the United States, and more than 11,600 patients died of this disease in 1998. Transitional cell carcinoma is less frequent, with an incidence of approximately 500 cases per year in the United States.

Surgery has been the primary therapy for renal cell adenocarcinoma for many decades. Until recently, metastatic disease has been refractory to any systemic therapy. With recent developments in systemic therapies, particularly immunotherapies, metastatic renal cell carcinoma may be approached aggressively in appropriate patients with a possibility of durable responses. Nevertheless, there is a remaining need for effective therapies for these patients.

Of all new cases of cancer in the United States, bladder cancer represents approximately 5 percent in men (fifth most common neoplasm) and 3 percent in women (eight most common neoplasm). The incidence is increasing slowly, concurrent with an increasing older population. In 1998, there was an estimated 54,500 cases, including 39,500 in men and 15,000 in women. The age-adjusted incidence in the United States is 32 per 100,000 for men and 8 per 100,000 in women. The historic male/female ratio of 3:1 may be decreasing related to smoking patterns in women. There were an estimated 11,000 deaths from bladder cancer in 1998 (7,800 in men and 3,900 in women). Bladder cancer incidence and mortality strongly increase with age and will be an increasing problem as the population becomes more elderly.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

An estimated 130,200 cases of colorectal cancer occurred in 2000 in the United States, including 93,800 cases of colon cancer and 36,400 of rectal cancer. Colorectal cancers are the third most common cancers in men and women. Incidence rates declined significantly during 992-1996 (−2.1% per year). Research suggests that these declines have been due to increased screening and polyp removal, preventing progression of polyps to invasive cancers. There were an estimated 56,300 deaths (47,700 from colon cancer, 8,600 from rectal cancer) in 2000, accounting for about 11% of all U.S. cancer deaths.

At present, surgery is the most common form of therapy for colorectal cancer, and for cancers that have not spread, it is frequently curative. Chemotherapy, or chemotherapy plus radiation is given before or after surgery to most patients whose cancer has deeply perforated the bowel wall or has spread to the lymph nodes. A permanent colostomy (creation of an abdominal opening for elimination of body wastes) is occasionally needed for colon cancer and is infrequently required for rectal cancer. There continues to be a need for effective diagnostic and treatment modalities for colorectal cancer.

There were an estimated 164,100 new cases of lung and bronchial cancer in 2000, accounting for 14% of all U.S. cancer diagnoses. The incidence rate of lung and bronchial cancer is declining significantly in men, from a high of 86.5 per 100,000 in 1984 to 70.0 in 1996. In the 1990s, the rate of increase among women began to slow. In 1996, the incidence rate in women was 42.3 per 100,000.

Lung and bronchial cancer caused an estimated 156,900 deaths in 2000, accounting for 28% of all cancer deaths. During 1992-1996, mortality from lung cancer declined significantly among men (−1.7% per year) while rates for women were still significantly increasing (0.9% per year). Since 1987, more women have died each year of lung cancer than breast cancer, which, for over 40 years, was the major cause of cancer death in women. Decreasing lung cancer incidence and mortality rates most likely resulted from decreased smoking rates over the previous 30 years; however, decreasing smoking patterns among women lag behind those of men. Of concern, although the declines in adult tobacco use have slowed, tobacco use in youth is increasing again.

Treatment options for lung and bronchial cancer are determined by the type and stage of the cancer and include surgery, radiation therapy, and chemotherapy. For many localized cancers, surgery is usually the treatment of choice. Because the disease has usually spread by the time it is discovered, radiation therapy and chemotherapy are often needed in combination with surgery. Chemotherapy alone or combined with radiation is the treatment of choice for small cell lung cancer; on this regimen, a large percentage of patients experience remission, which in some cases is long lasting. There is however, an ongoing need for effective treatment and diagnostic approaches for lunch and bronchial cancers.

An estimated 182,800 new invasive cases of breast cancer were expected to occur among women in the United States during 2000. Additionally, about 1,400 new cases of breast cancer were expected to be diagnosed in men in 2000. After increasing about 4% per year in the 1980s, breast cancer incidence rates in women have leveled off in the 1990s to about 110.6 cases per 100,000.

In the U.S. alone, there were an estimated 41,200 deaths (40,800 women, 400 men) in 2000 due to breast cancer. Breast cancer ranks second among cancer deaths in women. According to the most recent data, mortality rates declined significantly during 1992-1996 with the largest decreases in younger women, both white and black. These decreases were probably the result of earlier detection and improved treatment.

Taking into account the medical circumstances and the patient's preferences, treatment of breast cancer may involve lumpectomy (local removal of the tumor) and removal of the lymph nodes under the arm; mastectomy (surgical removal of the breast) and removal of the lymph nodes under the arm; radiation therapy; chemotherapy; or hormone therapy. Often, two or more methods are used in combination. Numerous studies have shown that, for early stage disease, long-term survival rates after lumpectomy plus radiotherapy are similar to survival rates after modified radical mastectomy. Significant advances in reconstruction techniques provide several options for breast reconstruction after mastectomy. Recently, such reconstruction has been done at the same time as the mastectomy.

Local excision of ductal carcinoma in situ (DCIS) with adequate amounts of surrounding normal breast tissue may prevent the local recurrence of the DCIS. Radiation to the breast and/or tamoxifen may reduce the chance of DCIS occurring in the remaining breast tissue. This is important because DCIS, if left untreated, may develop into invasive breast cancer. Nevertheless, there are serious side effects or sequelae to these treatments. There is, therefore, a need for efficacious breast cancer treatments.

There were an estimated 23,100 new cases of ovarian cancer in the United States in 2000. It accounts for 4% of all cancers among women and ranks second among gynecologic cancers. During 1992-1996, ovarian cancer incidence rates were significantly declining. Consequent to ovarian cancer, there were an estimated 14,000 deaths in 2000. Ovarian cancer causes more deaths than any other cancer of the female reproductive system.

Surgery, radiation therapy, and chemotherapy are treatment options for ovarian cancer. Surgery usually includes the removal of one or both ovaries, the fallopian tubes (salpingo-oophorectomy), and the uterus (hysterectomy). In some very early tumors, only the involved ovary will be removed, especially in young women who wish to have children. In advanced disease, an attempt is made to remove all intra-abdominal disease to enhance the effect of chemotherapy. There continues to be an important need for effective treatment options for ovarian cancer.

There were an estimated 28,300 new cases of pancreatic cancer in the United States in 2000. Over the past 20 years, rates of pancreatic cancer have declined in men. Rates among women have remained approximately constant but may be beginning to decline. Pancreatic cancer caused an estimated 28,200 deaths in 2000 in the United States. Over the past 20 years, there has been a slight but significant decrease in mortality rates among men (about −0.9% per year) while rates have increased slightly among women.

Surgery, radiation therapy, and chemotherapy are treatment options for pancreatic cancer. These treatment options can extend survival and/or relieve symptoms in many patients but are not likely to produce a cure for most. There is a significant need for additional therapeutic and diagnostic options for pancreatic cancer.

### SUMMARY OF THE INVENTION

The present invention relates to a novel gene, designated 103P3E8, that is over-expressed in multiple cancers listed in Table I. Northern blot expression analysis of 103P3E8 gene expression in normal tissues shows a restricted expression pattern in adult tissues. The nucleotide (FIG. 2 or FIG. 4) and amino acid (FIG. 2, FIG. 3 and FIG. 4) sequences of 103P3E8 are provided. The tissue-related profile of 103P3E8 in normal adult tissues, combined with the over-expression observed in prostate and other tumors, shows that 103P3E8 is aberrantly over-expressed in at least some cancers, and thus serves as a useful diagnostic and/or therapeutic target for cancers of the tissues such as those listed in Table I.

The invention provides polynucleotides corresponding or complementary to all or part of the 103P3E8 genes, mRNAs, and/or coding sequences, preferably in isolated form, including polynucleotides encoding 103P3E8-related proteins and fragments of 4, 5, 6, 7, 8, 9, 10, 11, 12, 13, 14, 15, 16, 17, 18, 19, 20, 21, 22, 23, 24, 25, or more than 25 contiguous amino acids; at least 30, 35, 40, 45, 50, 55, 60, 65, 70, 80, 85, 90, 95, 100 or more than 100 contiguous amino acids of a 103P3E8-related protein, as well as the peptides/proteins themselves; DNA, RNA, DNA/RNA hybrids, and related molecules, polynucleotides or oligonucleotides complementary or having at least a 90% homology to the 103P3E8 genes or mRNA sequences or parts thereof, and polynucleotides or oligonucleotides that hybridize to the 103P3E8 genes, mRNAs, or to 103P3E8-encoding polynucleotides. Also provided are means for isolating cDNAs and the genes encoding 103P3E8. Recombinant DNA molecules containing 103P3E8 polynucleotides, cells transformed or transduced with such molecules, and host-vector systems for the expression of 103P3E8 gene products are also provided. The invention further provides antibodies that bind to 103P3E8 proteins and polypeptide fragments thereof, including polyclonal and monoclonal antibodies, murine and other mammnalian antibodies, chimeric antibodies, humanized and fully human antibodies, and antibodies labeled with a detectable marker.

The invention further provides methods for detecting the presence and status of 103P3E8 polynucleotides and proteins in various biological samples, as well as methods for identifying cells that express 103P3E8. A typical embodiment of this invention provides methods for monitoring 103P3E8 gene products in a tissue or hematology sample having or suspected of having some form of growth dysregulation such as cancer.

The invention further provides various immunogenic or therapeutic compositions and strategies for treating cancers that express 103P3E8 such as prostate cancers, including therapies aimed at inhibiting the transcription, translation, processing or function of 103P3E8 as well as cancer vaccines.

### BRIEF DESCRIPTION OF THE FIGURES

FIG. 1 shows the 103P3E8 suppression subtractive hybridization (SSH) DNA sequence that is 367 nucleotides in length.

FIG. 2A-B. The cDNA and amino acid sequence of 103P3E8.

FIG. 3. The amino acid sequence encoded by the open reading frame of the nucleic acid sequence set forth in FIG. 2. Underlined region (amino acids 179-798) corresponds to the amino acid sequence present in clone 7, FIG. 4A and FIG. 4B.

FIG. 4A-D. A 2251 base pair portion of the nucleic (and corresponding 528 amino acid sequence) of FIG. 2. FIG. 4A-B: 103P3E8 clone 7 cDNA sequence and translation. FIG. 4C-D: 103P3E8 clone 7 cDNA renumbered to coincide with FIG. 2.

FIG. 5 A-C. Results of a Northern blot analysis of 103P3E8 expression in various normal human tissues (using the 103P3E8 SSH fragment as a probe) and LAPC xenografts. Two multiple tissue northern blots (Clontech) with 2 μg of mRNA/lane, and LAPC xenograft northern blots with 10 μg of total RNA/lane were probed with the 103P3E8 SSH fragment. Size standards in kilobases (kb) are indicated on the side. The results show predominant expression of a 5 kb 103P3E8 transcript in prostate and the prostate cancer xenografts. FIG. 5A: Lanes represent 1. Heart, 2. Brain, 3. Placenta, 4. Lung, 5. Liver, 6. Skeletal Muscle, 7. Kidney, 8. Pancreas. FIG. 5B: Lanes represent 1) Spleen, 2) Thymus, 3) Prostate, 4) Testis, 5) Ovary, 6) Small Intestine, 7) Colon, 8) Leukocytes. FIG. 5C: Lanes represent 1) Prostate, 2) LAPC-4 AD, 3) LAPC-4 AI, 4) LAPC-9 AD, 5) LAPC-9 AI.

FIG. 6. Expression of 103P3E8 in LAPC xenografts. RNA was extracted from the LAPC xenografts that were grown subcutaneously (sc) or intra-tibially (it) within the mouse bone. Northern blots with 10 μg of total RNA/lane were probed with the 103P3E8 SSH fragment. Size standards in kilobases (kb) are indicated on the side. Lanes represent: 1) LAPC4 AD sc, 2) LAPC-4 AD sc, 3) LAPC-4 AD sc, 4) LAPC-4 AD it, 5) LAPC-4 AD it, 6) LAPC-4 AD it, 7) LAPC-4 AD 2,8) LAPC-9 AD sc, 9) LAPC-9 AD sc, 10) LAPC-9 AD it, 11) LAPC-9 AD it, 12) LAPC-9 AD it, 13) LAPC-3 AI sc, 14) LAPC-3 AI sc.

FIG. 7A-C. Northern blot analysis of 103P3E8 expression in prostate and multiple cancer cell lines. RNA was extracted from the LAPC xenograft and a number of cancer cell lines. Northern blots with 10 μg of total RNA/lane were probed with the 103P3E8 SSH fragment. Size standards in kilobases (kb) are indicated on the side. Lanes represent: 1) LAPC-4 AD, 2) LAPC-4 AI, 3) LAPC-9 AD, 4) LAPC-9 AI, 5) TSUPR-1, 6) DU145, 7) LNCaP, 8) PC-3, 9) LAPC-4 CL, 10) PrEC, 11) HT1197, 12) SCABER, 13) UM-UC-3, 14) TCCSUP, 15) J82, 16) 5637, 17) 293T, 18) RD-ES, 19) PANC-1, 20) BxPC-3, 21) HPAC, 22) Capan-1, 23) CaCo-2, 24) LoVo, 25) T84, 26) Colo-205, 27) KCL 22, 28) PFSK-1, 29) T98G, 30) SK-ES-1, 31) HOS, 32) U2-OS, 33) RD-ES, 34) CALU-1, 35) A427, 36) NCI-H82, 37) NCI-H146, 38) 769-P, 39) A498, 40) CAKI-1, 41) SW839, 42) BT20, 43) CAMA-1, 44) DU4475, 45) MCF-7, 46) MDA-MB-435s, 47) NTERRA-2, 48) NCCIT, 49) TERA-1, 50) TERA-2, 51) A431, 52) HeLa, 53) OV-1063, 54) PA-1, 55) SW626,56) CAOV-3.

FIG. 8. Northern blot analysis of 103P3E8 expression in prostate cancer patient samples. RNA was extracted from the prostate tumors and their normal adjacent tissue derived from prostate cancer patients. Northern blots with 10 μg of total RNA/lane were probed with the 103P3E8 SSH fragment. Size standards in kilobases (kb) are indicated on the side. Lanes represent: 1) Patient 1, normal adjacent tissue;

2) Patient 1, Gleason 9 tumor; 3) Patient 2, normal adjacent tissue; 4) Patient 2, Gleason 7 tumor; 5) Patient 3, normal adjacent tissue ; 6) Patient 3, Gleason 7 tumor.

FIG. 9. Expression of 103P3E8 in human cancers by RT-PCR. First strand cDNA was prepared from vital pool 1 (VP 1: liver, lung and kidney), vital pool 2 (VP2, pancreas, spleen and stomach), LAPC xenograft pool (LAPC-4AD, LAPC-4AI, LAPC-9AD and LAPC-9AI), prostate cancer pool, bladder cancer pool, kidney cancer pool, colon cancer pool and lung cancer pool. Normalization was performed by PCR using primers to actin and GAPDH. Semi-quantitative PCR, using primers to 103P3E8, was performed at 26 and 30 cycles of amplification. Results show expression of 103P3E8 in all tumor pools tested, prostate, bladder, kidney, colon and lung. Lanes represent: 1) VP1, 2) VP2, 3) Xenograft pool, 4) Prostate cancer pool, 5) Bladder cancer pool, 6) Kidney cancer pool, 7) Colon cancer pool, 8) Lung cancer pool.

FIG. 10. Expression of 103P3E8 in colon cancer patient specimens. RNA was extracted from colon tumors (T) and their normal adjacent tissue (NAT) derived from colon cancer patients. Northern blots with 10 μg of total RNA/lane were probed with 103P3E8 sequences. Size standards in kilobases (kb) are indicated on the side. Results show expression of 103P3E8 in samples derived from all five patients. Also, 103P3E8 is expressed in 2 of the 3 cell lines tested, Colo 205 and LoVo. Pt.1, stage I; Pt.2, stage II; Pt.3, stage III; Pt.4, stage IV; Pt.5, stage IV. CL=Cell lines (Colo 205, LoVo, SK-CO-1).

FIG. 11. Expression of 103P3E8 in human patient cancer specimens and cancer cell lines.

Expression of 103P3E8 was assayed in a panel of human cancers (T) and their respective matched normal tissues (N) on RNA dot blots. 103P3E8 expression was seen in kidney, breast, prostate, colon, stomach and rectum cancers. The expression detected in normal adjacent tissues (isolated from diseased tissues), but not in normal tissues (isolated from healthy donors), may indicate that these tissues are not fully normal and that 103P3E8 may be expressed in early stage tumors. 103P3E8 was also found to be highly expressed in the lung cancer cell line A549. Cancer cell lines are: (from left to right) HeLa (cervical carcinoma), Daudi (Burkitt's lyniphoma), K562 (CML), HL-60 (PML), G361 (melanoma), A549 (lung carcinoma), MOLT-4 (lymnphoblastic leuk.), SW480 (colorectal carcinoma), Raji (Burkitt's lymphoma).

FIG. 12. Expression of 103P3E8 protein in 293T cells with recognition by an anti-103P3E8 polyclonal antibody.

FIG. 13A-B. Western analysis results that show expression of 103P3E8 in colon, ovarian, and kidney cancer.

FIG. 14A-D. Sequence alignment of 103P3E8 with G proteins and an intermediate filament protein using the BLAST function (NCBI). 14A: Alignment of 103P3E8 ORF with the C. elegans G protein AAB04568. 14B: Alignment of 103P3E8 ORF with the human G protein RAB8. 14C: Alignment of 103P3E8 ORF with the C. elegans intermediate filament protein AAB04569. 14D: Alignment of 103P3E8 ORF with the C. elegans EF-hand calcium binding protein.

### DETAILED DESCRIPTION OF THE INVENTION

Outline of Sections

I.) Definitions

II.) Properties of 103P3E8.

III.) 103P3E8 Polynucleotides

III.A.) Uses of 103P3E8 Polynucleotides

III.A.1.) Monitoring of Genetic Abnormalities

III.A.2.) Antisense Embodiments

III.A.3.) Primers and Primer Pairs

III.A.4.) Isolation of 103P3E8-Encoding Nucleic Acid Molecules

III.A.5.) Recombinant Nucleic Acid Molecules and Host-Vector Systems

IV.) 103P3E8-related Proteins

IV.A.) Motif-bearing Protein Embodiments

IV.B.) Expression of 103P3E8-related Proteins

IV.C.) Modifications of 103P3E8-related Proteins

IV.D.) Uses of 103P3E8-related Proteins

V.) 103P3E8 Antibodies

VI.) 103P3E8 Transgenic Animals

VII.) Methods for the Detection of 103P3E8

VIII.) Methods for Monitoring the Status of 103P3E8-related Genes and Their Products

IX.) Identification of Molecules That Interact With 103P3E8

X.) Therapeutic Methods and Compositions

X.A.) 103P3E8 as a Target for Antibody-Based Therapy

XB.) Anti-Cancer Vaccines

XI.) Inhibition of 103P3E8 Protein Function

XI.A.) Inhibition of 103P3E8 With Intracellular Antibodies

XI.B.) Inhibition of 103P3E8 with Recombinant Proteins

XI.C.) Inhibition of 103P3E8 Transcription or Translation

XI.D.) General Considerations for Therapeutic Strategies

XII.) KITS

### I.) Definitions:

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asyrmmetry or induration above the prostate base. Locally advanced prostate cancer is presently diagnosed pathologically following radical prostatectomy if the tumor invades or penetrates the prostatic capsule, extends into the surgical margin, or invades the semninal vesicles.

“Altering the native glycosylation pattern” is intended for purposes herein to mean deleting one or more carbohydrate moieties found in native sequence 103P3E8 (either by removing the underlying glycosylation site or by deleting the glycosylation by chemical and/or enzymatic means), and/or adding one or more glycosylation sites that are not present in the native sequence 103P3E8. In addition, the phrase includes qualitative changes in the glycosylation of the native proteins, involving a change in the nature and proportions of the various carbohydrate moieties present.

The term “analog” refers to a molecule which is structurally similar or shares similar or corresponding attributes with another molecule (e.g. a 103P3E8-related protein). For example an analog of the 103P3E8 protein can be specifically bound by an antibody or T cell that specifically binds to 103P3E8.

The term “antibody” is used in the broadest sense. Therefore an “antibody” can be naturally occurring or man-made such as monoclonal antibodies produced by conventional hybridoma technology. Anti-103P3E8 antibodies comprise monoclonal and polyclonal antibodies as well as fragments containing the antigen-binding domain and/or one or more complementarity determining regions of these antibodies.

As used herein, an “antibody fragment”is defined as at least a portion of the variable region of the immunoglobulin molecule that binds to its target, i.e., the antigen-binding region. In one embodiment it specifically covers single anti-103P3E8 antibodies and clones thereof (including agonist, antagonist and neutralizing antibodies) and anti-103P3E8 antibody compositions with polyepitopic specificity.

The term “codon optimized sequences” refers to nucleotide sequences that have been optimized for a particular host species by replacing any codons having a usage frequency of less than about 20%. Nucleotide sequences that have been optimized for expression in a given host species by elimination of spurious polyadenylation sequences, elimination of exon/intron splicing signals, elimination of transposon-like repeats and/or optimization of GC content in addition to codon optimization are referred to herein as an “expression enhanced sequences.”

The term “cytotoxic agent” as used herein refers to a substance that inhibits or prevents the function of cells and/or causes destruction of cells. The term is intended to include radioactive isotopes chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, ytrium, bismuth ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹², P³² and radioactive isotopes of Lu. Antibodies may also be conjugated to an anti-cancer pro-drug activating enzyme capable of converting the pro-drug to its active form.

The term “homolog” refers to a molecule which exhibits homology to another molecule, by for example, having sequences of chemical residues that are the same or similar at corresponding positions.

As used herein, the terms “hybridize”, “hybridizing”, “hybridizes” and the like, used in the context of polynucleotides, are meant to refer to conventional hybridization conditions, preferably such as hybridization in 50% formamide/6XSSC/0.1% SDS/100 μg/ml ssDNA, in which temperatures for hybridization are above 37 degrees C and temperatures for washing in 0.lXSSC/0.1% SDS are above 55 degrees C.

As used herein, a polynucleotide is said to be “isolated” when it is substantially separated from contaminant polynucleotides that correspond or are complementary to genes other than the 103P3E8 gene or that encode polypeptides other than 103P3E8 gene product or fragments thereof. A skilled artisan can readily employ nucleic acid isolation procedures to obtain an isolated 103P3E8 polynucleotide.

As used herein, a protein is said to be “isolated”when physical, mechanical or chemical methods are employed to remove the 103P3E8 protein from cellular constituents that are normally associated with the protein. A skilled artisan can readily employ standard purification methods to obtain an isolated 103P3E8 protein. Alternatively, an isolated protein can be prepared by chemical means.

The term “mammal” as used herein refers to any organism classified as a mammal, including mice, rats, rabbits, dogs, cats, cows, horses and humans. In one embodiment of the invention, the mammal is a mouse. In another embodiment of the invention, the mammal is a human.

As used herein, the terms “metastatic prostate cancer” and “metastatic disease” mean prostate cancers that have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy is a preferred treatment modality. Patients with metastatic prostate cancer eventually develop an androgen-refractory state within 12 to 18 months of treatment initiation. Approximately half of these androgen-refractory patients die within 6 months after developing that status. The most common site for prostate cancer metastasis is bone. Prostate cancer bone metastases are often osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

The term “monoclonal antibody” as used herein refers to an antibody obtained from a population of substantially homogeneous antibodies, i.e., the antibodies comprising the population are identical except for possible naturally occurring mutations that are present in minor amounts.

As used herein “motif” as in biological motif of an 103P3E8-related protein, refers to any pattern of amino acids forming part of the primary sequence of a protein, that is associated with a particular function (e.g. protein-protein interaction, protein-DNA interaction, etc) or modification (e.g. that is phosphorylated, glycosylated or amidated), or localization (e.g. secretory sequence, nuclear localization sequence, etc.) or a sequence that is correlated with being immunogenic, either humorally or cellularly. A motif can be either contiguous or capable of being aligned to certain positions that are generally correlated with a certain function or property.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T) (as shown for example in SEQ ID NO: 1) can also be uracil (U); this definition pertains to the differences between the chemical structures of DNA and RNA, in particular the observation that one of the four major bases in RNA is uracil (U) instead of thymidine (T).

As used herein, the term “polypeptide” means a polymer of at least about 4, 5, 6, 7, or 8 amino acids. Throughout the specification, standard three letter or single letter designations for amino acids are used. In the art, this term is often used interchangeably with “peptide” or “protein”.

As used herein, a “recombinant” DNA or RNA molecule is a DNA or RNA molecule that has been subjected to molecular manipulation in vitro.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are identified by, but not limited to, those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

A “transgenic animal” (e.g., a mouse or rat) is an animal having cells that contain a transgene, which transgene was introduced into the animal or an ancestor of the animal at a prenatal, e.g., an embryonic stage. A “transgene” is a DNA that is integrated into the genome of a cell from which a transgenic animal develops.

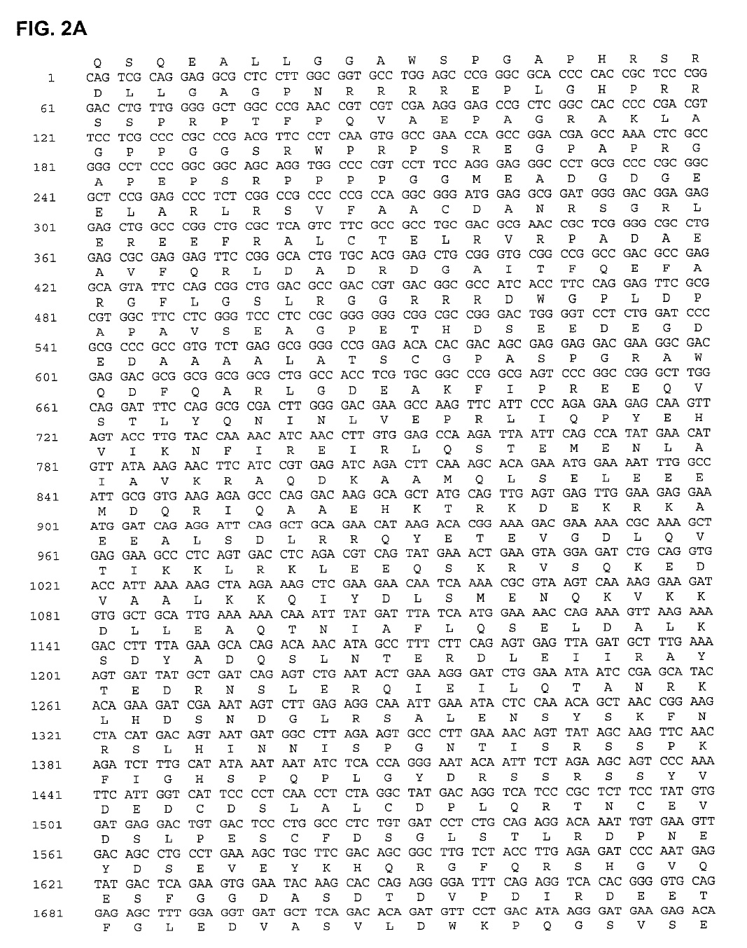

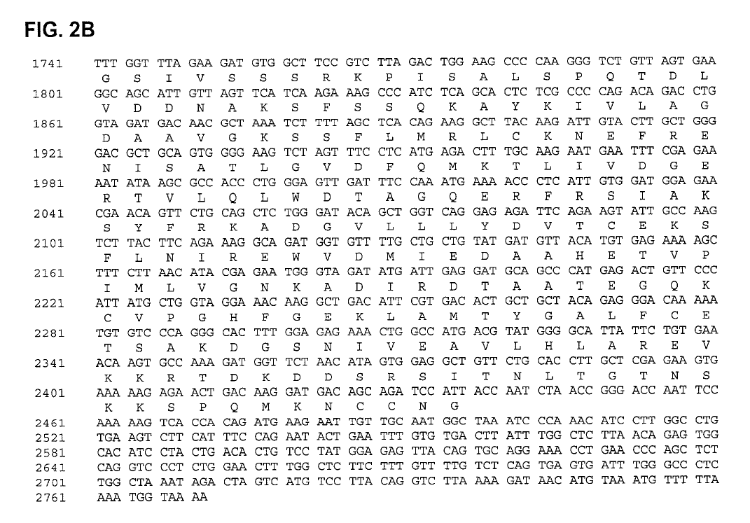

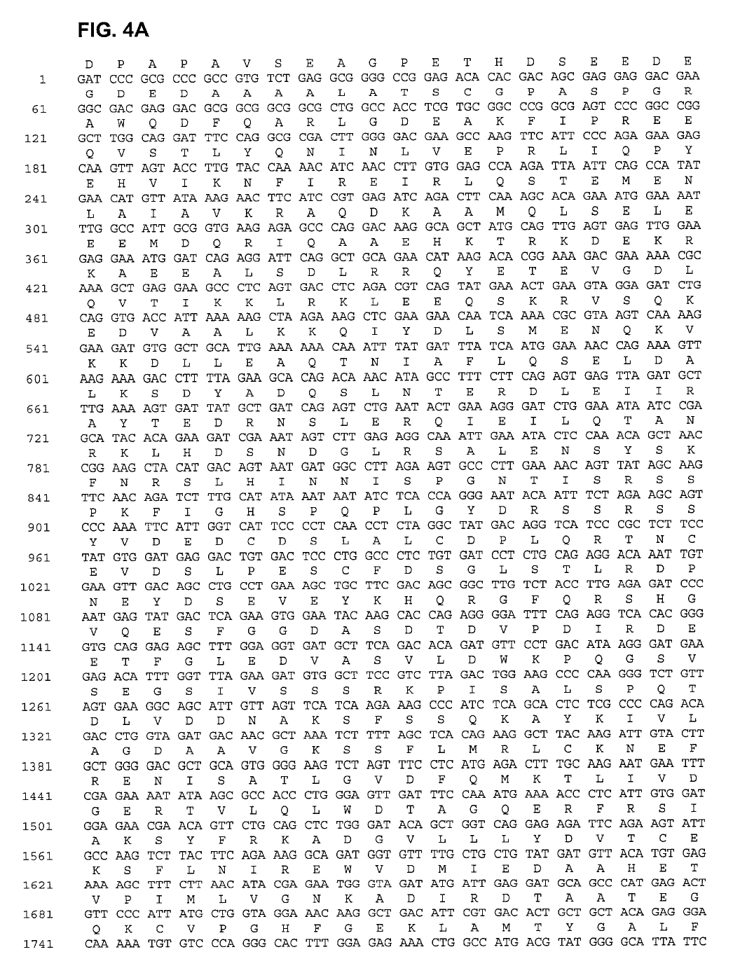

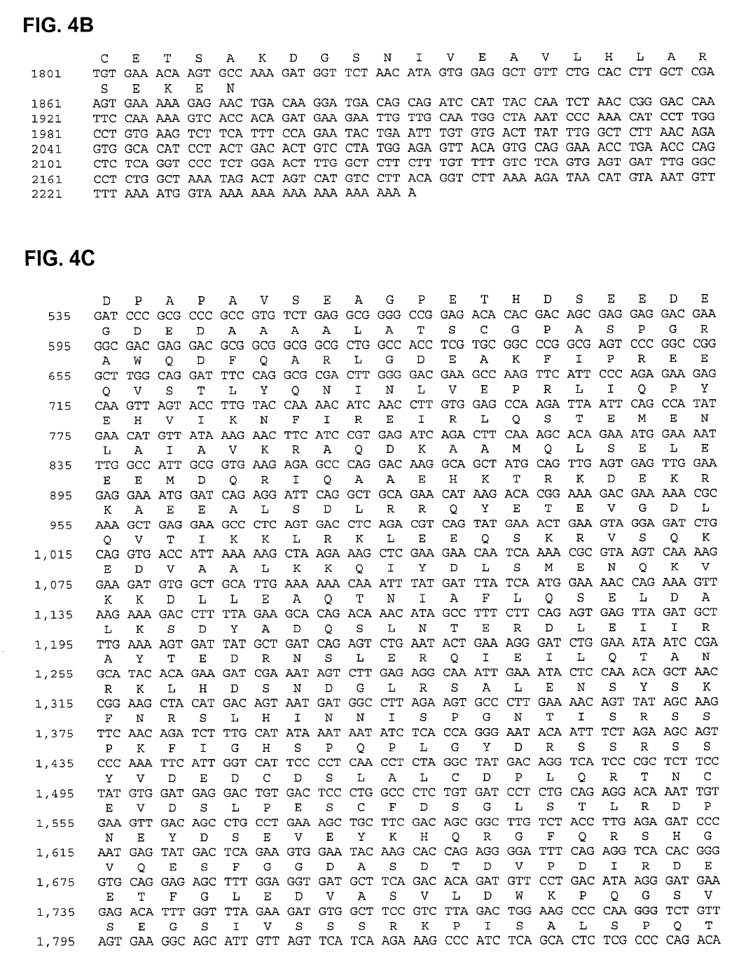

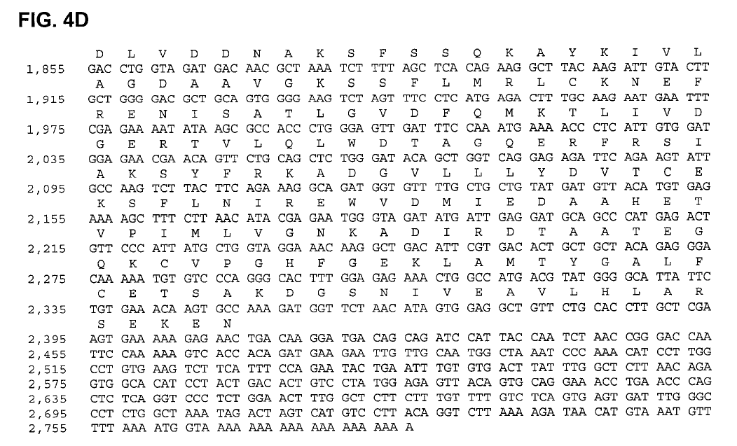

The term “variant” refers to a molecule that exhibits a variation from a described type or norm, such as a protein that has one or more different amino acid residues in the corresponding position(s) of a specifically described protein (e.g. the 103P3E8 protein shown in FIG. 2 or FIG. 4). An analog is an example of a variant protein.

As used herein, the 103P3E8-related gene and 103P3E8-related protein includes the 103P3E8 genes and proteins specifically described herein, as well as structurally and/or functionally similar variants or analog of the foregoing. 103P3E8 peptide analogs generally share at least about 50%, 60%, 70%, 80%, 90% or more amino acid homology (using BLAST criteria). 103P3E8 nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

The 103P3E8-related proteins of the invention include those specifically identified herein, as well as allelic variants, conservative substitution variants, analogs and homologs that can be isolated/generated and characterized without undue experimentation following the methods outlined herein or readily available in the art. Fusion proteins that combine parts of different 103P3E8 proteins or fragments thereof, as well as fusion proteins of a 103P3E8 protein and a heterologous polypeptide are also included. Such 103P3E8 proteins are collectively referred to as the 103P3E8-related proteins, the proteins of the invention, or 103P3E8. As used herein, the term “103P3E8-related protein” refers to a polypeptide fragment or an 103P3E8 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100 or more than 100 amino acids.

### II.) Properties of 103P3E8.

As disclosed herein, 103P3E8 exhibits specific properties that are analogous to those found in a family of molecules whose polynucleotides, polypeptides, reactive cytotoxic T cells (CTL), reactive helper T cells (HTL) and anti-polypeptide antibodies are used in well known diagnostic assays that examine conditions associated with dysregulated cell growth such as cancer, in particular prostate cancer (see, e.g., both its highly specific pattern of tissue expression as well as its overexpression in certain cancers as described for example in Example 4). The best-known member of this class is PSA, the archetypal marker that has been used by medical practitioners for years to identify and monitor the presence of prostate cancer (see, e.g., Merrill et al., J. Urol. 163(2): 503-5120 (2000); Polascik et al., J. Urol. Aug; 162(2):293-306 (1999) and Fortier et al., J. Nat. Cancer Inst. 91(19): 1635-1640(1999)). A variety of other diagnostic markers are also used in this context including p53 and K-ras (see, e.g., Tulchinsky et al., Int J Mol Med Jul. 4, 1999 (1):99-102 and Minimoto et al., Cancer Detect Prev 2000;24(1): 1-12). Therefore, this disclosure of the 103P3E8 polynucleotides and polypeptides (as well as the 103P3E8 polynucleotide probes and anti-103P3E8 antibodies used to identify the presence of these molecules) and their properties allows skilled artisans to utilize these molecules in methods that are analogous to those used, for example, in a variety of diagnostic assays directed to examining conditions associated with cancer.

Typical embodiments of diagnostic methods which utilize the 103P3E8 polynucleotides, polypeptides, reactive T cells and antibodies are analogous to those methods from well-established diagnostic assays which employ, e.g., PSA polynucleotides, polypeptides, reactive T cells and antibodies. For example, just as PSA polynucleotides are used as probes (for example in Northern analysis, see, e.g., Sharief et al., Biochem. Mol. Biol. Int. 33(3):567-74(1994)) and primers (for example in PCR analysis, see, e.g., Okegawa et al., J. Urol. 163(4): 1189-1190 (2000)) to observe the presence and/or the level of PSA mRNAs in methods of monitoring PSA overexpression or the metastasis of prostate cancers, the 103P3E8 polynucleotides described herein can be utilized in the same way to detect 103P3E8 overexpression or the metastasis of prostate and other cancers expressing this gene. Alternatively, just as PSA polypeptides are used to generate antibodies specific for PSA which can then be used to observe the presence and/or the level of PSA proteins in methods to monitor PSA protein overexpression (see, e.g., Stephan et al., Urology 55(4):560-3 (2000)) or the metastasis of prostate cells (see, e.g., Alanen et al., Pathol. Res. Pract. 192(3):233-7 (1996)), the 103P3E8 polypeptides described herein can be utilized to generate antibodies for use in detecting 103P3E8 overexpression or the metastasis of prostate cells and cells of other cancers expressing this gene.

Specifically, because metastases involves the movement of cancer cells from an organ of origin (such as the lung or prostate gland etc.) to a different area of the body (such as a lymph node), assays which examine a biological sample for the presence of cells expressing 103P3E8 polynucleotides and/or polypeptides can be used to provide evidence of metastasis. For example, when a biological sample from tissue that does not normally contain 103P3E8-expressing cells (lymph node) is found to contain 103P3E8-expressing cells such as the 103P3E8 expression seen in LAPC4 and LAPC9, xenografts isolated from lymph node and bone metastasis, respectively, this finding is indicative of metastasis.

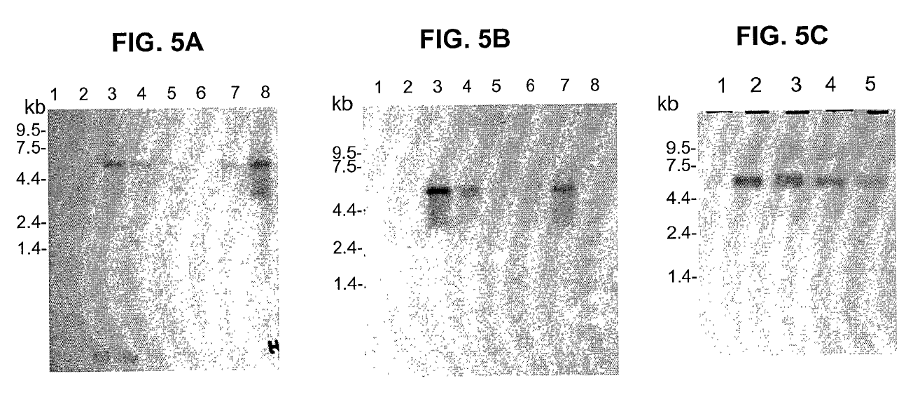

Alternatively 103P3E8 polynucleotides and/or polypeptides can be used to provide evidence of cancer, for example, when cells in a biological sample that do not normally express 103P3E8 or express 103P3E8 at a different level are found to express 103P3E8 or have an increased expression of 103P3E8 (see, e.g., the 103P3E8 expression in kidney, lung and colon cancer cells and in patient samples etc. shown in FIGS. 5-11). In such assays, artisans may further wish to generate supplementary evidence of metastasis by testing the biological sample for the presence of a second tissue restricted marker (in addition to 103P3E8) such as PSA, PSCA etc. (see, e.g., Alanen et al., Pathol. Res. Pract. 192(3): 233-237 (1996)).

Just as PSA polynucleotide fragments and polynucleotide variants are employed by skilled artisans for use in methods of monitoring PSA, 103P3E8 polynucleotide fragments and polynucleotide variants are used in an analogous manner. In particular, typical PSA polynucleotides used in methods of monitoring PSA are probes or primers which consist of fragments of the PSA cDNA sequence. Illustrating this, primers used to PCR amplify a PSA polynucleotide must include less than the whole PSA sequence to function in the polymerase chain reaction. In the context of such PCR reactions, skilled artisans generally create a variety of different polynucleotide fragments that can be used as primers in order to amplify different portions of a polynucleotide of interest or to optimize amplification reactions (see, e.g., Caetano-Anolles, G. Biotechniques 25(3): 472-476, 478-480 (1998); Robertson et al., Methods Mol. Biol. 98:121-154 (1998)). An additional illustration of the use of such fragments is provided in Example 4, where a 103P3E8 polynucleotide fragment is used as a probe to show the expression of 103P3E8 RNAs in cancer cells. In addition, variant polynucleotide sequences are typically used as primers and probes for the corresponding mRNAs in PCR and Northern analyses (see, e.g., Sawai et al., Fetal Diagn. Ther. Nov.-Dec. 11, 1996 (6):407-13 and Current Protocols In Molecular Biology, Volume 2, Unit 2, Frederick M. Ausubel et al. eds., 1995)). Polynucleotide fragments and variants are useful in this context where they are capable of binding to a target polynucleotide sequence (e.g. the 103P3E8 polynucleotide shown in SEQ ID NO: 1) under conditions of high stringency.

Furthermore, PSA polypeptides which contain an epitope that can be recognized by an antibody or T cell that specifically binds to that epitope are used in methods of monitoring PSA. 103P3E8 polypeptide fragments and polypeptide analogs or variants can also be used in an analogous manner. This practice of using polypeptide fragments or polypeptide variants to generate antibodies (such as anti-PSA antibodies or T cells) is typical in the art with a wide variety of systems such as fusion proteins being used by practitioners (see, e.g., Current Protocols In Molecular Biology, Volume 2, Unit 16, Frederick M. Ausubel et al. eds., 1995). In this context, each epitope(s) functions to provide the architecture with which an antibody or T cell is reactive. Typically, skilled artisans create a variety of different polypeptide fragments that can be used in order to generate immune responses specific for different portions of a polypeptide of interest (see, e.g., U.S. Pat. No. 5,840,501 and U.S. Pat. No. 5,939,533). For example it may be preferable to utilize a polypeptide comprising one of the 103P3E8 biological motifs discussed herein or available in the art. Polypeptide fragments, variants or analogs are typically useful in this context as long as they comprise an epitope capable of generating an antibody or T cell specific for a target polypeptide sequence (e.g. the 103P3E8 polypeptide shown in SEQ ID NO: 2).

As shown herein, the 103P3E8 polynucleotides and polypeptides (as well as the 103P3E8 polynucleotide probes and anti-103P3E8 antibodies or T cells used to identify the presence of these molecules) exhibit specific properties that make them useful in diagnosing cancers of the prostate. Diagnostic assays that measure the presence of 103P3E8 gene products, in order to evaluate the presence or onset of a disease condition described herein, such as prostate cancer, are used to identify patients for preventive measures or further monitoring, as has been done so successfully with PSA. Moreover, these materials satisfy a need in the art for molecules having similar or complementary characteristics to PSA in situations where, for example, a definite diagnosis of metastasis of prostatic origin cannot be made on the basis of a test for PSA alone (see, e.g., Alanen et al., Pathol. Res. Pract. 192(3): 233-237 (1996)), and consequently, materials such as 103P3E8 polynucleotides and polypeptides (as well as the 103P3E8 polynucleotide probes and anti-103P3E8 antibodies used to identify the presence of these molecules) must be employed to confirm metastases of prostatic origin.

Finally, in addition to their use in diagnostic assays, the 103P3E8 polynucleotides disclosed herein have a number of other specific utilities such as their use in the identification of oncogenetic associated chromosomal abnormalities in the chromosomal region to which the 103P3E8 gene maps (see Example 3 below). Moreover, in addition to their use in diagnostic assays, the 103P3E8-related proteins and polynucleotides disclosed herein have other utilities such as their use in the forensic analysis of tissues of unknown origin (see, e.g., Takahama K Forensic Sci Int Jun. 28, 1996; 80(1-2): 63-9).

Additionally, 103P3E8-related proteins or polynucleotides of the invention can be used to treat a pathologic condition characterized by the over-expression of 103P3E8. For example, the amino acid or nucleic acid sequence of FIG. 2, FIG. 4, or fragments of either, can be used to generate an immune response to the 103P3E8 antigen. Antibodies or other molecules that react with 103P3E8 can be used to modulate the function of this molecule, and thereby provide a therapeutic benefit.

### III.) 103P3E8 Polynucleotides

One aspect of the invention provides polynucleotides corresponding or complementary to all or part of an 103P3E8 gene, mRNA, and/or coding sequence, preferably in isolated form, including polynucleotides encoding an 103P3E8-related protein and fragments thereof, DNA, RNA, DNA/RNA hybrid, and related molecules, polynucleotides or oligonucleotides complementary to an 103P3E8 gene or mRNA sequence or a part thereof, and polynucleotides or oligonucleotides that hybridize to an 103P3E8 gene, mRNA, or to an 103P3E8 encoding polynucleotide (collectively, “103P3E8 polynucleotides”). In all instances when referred to in this section, T can also be U in FIG. 2 or FIG. 4.

Embodiments of a 103P3E8 polynucleotide include: a 103P3E8 polynucleotide having the sequence shown in FIG. 2 or FIG. 4, the nucleotide sequence of 103P3E8 as shown in FIG. 2 or FIG. 4, wherein T is U; at least 10 contiguous nucleotides of a polynucleotide having the sequence as shown in FIG. 2 or FIG. 4; or, at least 10 contiguous nucleotides of a polynucleotide having the sequence as shown in FIG. 2 or FIG. 4 where T is U. Further 103P3E8 nucleotides comprise, where T can be U:

(a) a polynucleotide of at least 10 bases of the sequence as shown in FIG. 2 (SEQ ID NO: 1), comprising that portion which encodes amino acids 1 to 178, 1 to 179, 179 to 276, 180 to 276, 277 to 798, 799 to 832, 1-832, 179 to 798, 180 to 798, 471 to 832, or 93 to 832; and

(b) a polynucleotide that selectively hybridizes under stringent conditions to a polynucleotide of (a).

As used herein, a range is understood to specifically disclose all whole unit positions thereof. Moreover, a peptide that is encoded by any of the foregoing is also within the scope of the invention. An alternative embodiment comprises a polynucleotide of the invention with a proviso that the nucleic acid does not include one or more of the specified positions or ranges.

Also within the scope of the invention is a nucleotide, as well as any peptide encoded thereby, that starts at any of the following positions and ends at a higher position or range, the positions corresponding to the nucleotides encoding the following 103P3E8 amino acid residues: 1 to 178, 1 to 179, 179 to 276, 180 to 276, 277 to 798, 799 to 832, 1-832, 179 to 798, 180 to 798, 471 to 832, and 93 to 832; wherein a range as used in this section is understood to specifically disclose all whole unit positions thereof.

Another embodiment of the invention comprises a polynucleotide that encodes a 103P3E8-related protein whose sequence is encoded by the cDNA contained in the plasmid deposited with American Type Culture Collection (ATCC) as plasmid p103P3E8-7, assigned Designation No. PTA-1262. Another embodiment comprises a polynucleotide that hybridizes under stringent hybridization conditions, to the human 103P3E8 cDNA shown in FIG. 2 or FIG. 4 or to a polynucleotide fragment of either.

Typical embodiments of the invention disclosed herein include 103P3E8 polynucleotides that encode specific portions of the 103P3E8 mRNA sequence (and those which are complementary to such sequences) such as those that encode the protein and fragments thereof, for example of 4, 5, 6, 7, 8, 9, 10, 11, 12, 13, 14, 15 or more contiguous amino acids.

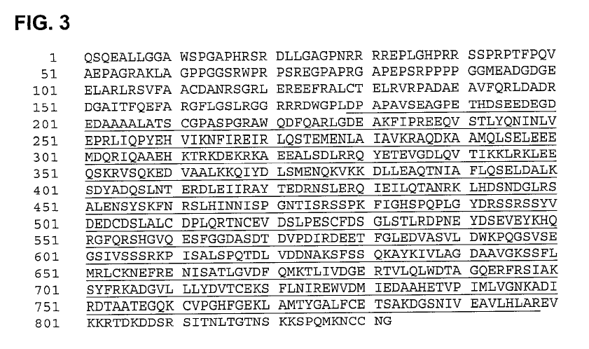

For example, representative embodiments of the invention disclosed herein include: polynucleotides and their encoded peptides themselves encoding about amino acid 1 to about amino acid 10 of the 103P3E8 protein shown in FIG. 2, FIG. 3 or FIG. 4, polynucleotides encoding about amino acid 10 to about amino acid 20 of the 103P3E8 protein shown in FIG. 2, FIG. 3 or FIG. 4, polynucleotides encoding about amino acid 20 to about amino acid 30 of the 103P3E8 protein shown in FIG. 2, FIG. 3 or FIG. 4, polynucleotides encoding about amino acid 30 to about amino acid 40 of the 103P3E8 protein shown in FIG. 2, FIG. 3 or FIG. 4, polynucleotides encoding about amino acid 40 to about amino acid 50 of the 103P3E8 protein shown in FIG. 2, FIG. 3 or FIG. 4, polynucleotides encoding about amino acid 50 to about amino acid 60 of the 103P3E8 protein shown in FIG. 2, FIG. 3 or FIG. 4, polynucleotides encoding about amino acid 60 to about amino acid 70 of the 103P3E8 protein shown in FIG. 2, FIG. 3 or FIG. 4, polynucleotides encoding about amino acid 70 to about amino acid 80 of the 103P3E8 protein shown in FIG. 2, FIG. 3 or FIG. 4, polynucleotides encoding about amino acid 80 to about amino acid 90 of the 103P3E8 protein shown in FIG. 2, FIG. 3 or FIG. 4, polynucleotides encoding about amino acid 90 to about amino acid 100 of the 103P3E8 protein shown in FIG. 2, FIG. 3 or FIG. 4, in increments of about 10 amino acids, ending at the carboxyl terminal amino acid set forth in FIG. 2, FIG. 3 or FIG. 4. Accordingly polynucleotides encoding portions of the amino acid sequence (of about 10 amino acids), of amino acids 100 through the carboxyl terminal amino acid of the 103P3E8 protein are embodiments of the invention. Wherein it is understood that each particular amino acid position discloses that position plus or minus five amino acid residues.

Polynucleotides encoding relatively long portions of the 103P3E8 protein are also within the scope of the invention. For example, polynucleotides encoding from about amino acid 1 (or 20 or 30 or 40 etc.) to about amino acid 20, (or 30, or 40 or 50 etc.) of the 103P3E8 protein shown in FIG. 2, FIG. 3 or FIG. 4 can be generated by a variety of techniques well known in the art. These polynucleotide fragments can include any portion of the 103P3E8 sequence as shown in FIG. 2, 3, or 4.

Additional illustrative embodiments of the invention disclosed herein include 103P3E8 polynucleotide fragments encoding one or more of the biological motifs contained within the 103P3E8 protein sequence, including one or more of the motif-bearing subsequences of the 103P3E8 protein set forth in Tables V-XIX. In another embodiment, typical polynucleotide fragments of the invention encode one or more of the regions of 103P3E8 that exhibit homology to a known molecule. In another embodiment of the invention, typical polynucleotide fragments can encode one or more of the 103P3E8 N-glycosylation sites, cAMP and cGMP-dependent protein kinase phosphorylation sites, casein kinase II phosphorylation sites or N-myristoylation site and amidation sites.

III.A.) Uses of 103P3E8 Polynucleotides

III.A.1.) Monitoring of Genetic Abnormalities

The polynucleotides of the preceding paragraphs have a number of different specific uses. The human 103P3E8 gene maps to the chromosomal location set forth in Example 3. For example, because the 103P3E8 gene maps to this chromosome, polynucleotides that encode different regions of the 103P3E8 protein are used to characterize cytogenetic abnormalities of this chromosomal locale, such as abnormalities that are identified as being associated with various cancers. In certain genes, a variety of chromosomal abnormalities including rearrangements have been identified as frequent cytogenetic abnormalities in a number of different cancers (see e.g. Krajinovic et al., Mutat. Res. 382(3-4): 81-83 (1998); Johansson et al., Blood 86(10): 3905-3914 (1995) and Finger et al., P.N.A.S. 85(23): 9158-9162 (1988)). Thus, polynucleotides encoding specific regions of the 103P3E8 protein provide new tools that can be used to delineate, with greater precision than previously possible, cytogenetic abnormalities in the chromosomal region that encodes 103P3E8 that may contribute to the malignant phenotype. In this context, these polynucleotides satisfy a need in the art for expanding the sensitivity of chromosomal screening in order to identify more subtle and less common chromosomal abnormalities (see e.g. Evans et al., Am. J. Obstet. Gynecol 171(4): 1055-1057 (1994)).

Furthermore, as 103P3E8 was shown to be highly expressed in prostate and other cancers, 103P3E8 polynucleotides are used in methods assessing the status of 103P3E8 gene products in normal versus cancerous tissues. Typically, polynucleotides that encode specific regions of the 103P3E8 protein are used to assess the presence of perturbations (such as deletions, insertions, point mutations, or alterations resulting in a loss of an antigen etc.) in specific regions of the 103P3E8 gene, such as such regions containing one or more motifs. Exemplary assays include both RT-PCR assays as well as single-strand conformation polymorphism (SSCP) analysis (see, e.g., Marrogi et al., J. Cutan. Pathol. 26(8): 369-378 (1999), both of which utilize polynucleotides encoding specific regions of a protein to examine these regions within the protein.

III.A.2.) Antisense Embodiments

Other specifically contemplated nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of 103P3E8. For example, antisense molecules can be RNAs or other molecules, including peptide nucleic acids (PNAs) or non-nucleic acid molecules such as phosphorothioate derivatives, that specifically bind DNA or RNA in a base pair-dependent manner. A skilled artisan can readily obtain these classes of nucleic acid molecules using the 103P3E8 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, e.g., 103P3E8. See for example, Jack Cohen, Oligodeoxynucleotides, Antisense Inhibitors of Gene Expression, CRC Press, 1989; and Synthesis 1:1-5 (1988). The 103P3E8 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (0-oligo) in which a nonbridging oxygen atom of the phosphate group is replaced by a sulfur atom. The S-oligos of the present invention can be prepared by treatment of the corresponding 0-oligos with 3H- 1,2-benzodithiol-3-one-1,1-dioxide, which is a sulfur transfer reagent. See Iyer, R. P. et al, J. Org. Chem. 55:4693-4698 (1990); and Iyer, R. P. et al., J. Am. Chem. Soc. 112:1253-1254 (1990). Additional 103P3E8 antisense oligonucleotides of the present invention include morpholino antisense oligonucleotides known in the art (see, e.g., Partridge et al., 1996, Antisense & Nucleic Acid Drug Development 6: 169-175).

The 103P3E8 antisense oligonucleotides of the present invention typically can be RNA or DNA that is complementary to and stably hybridizes with the first 100 5′ codons or last 100 3′ codons of the 103P3E8 genomic sequence or the corresponding mRNA. Absolute complementarity is not required, although high degrees of complementarity are preferred. Use of an oligonucleotide complementary to this region allows for the selective hybridization to 103P3E8 MRNA and not to mRNA specifying other regulatory subunits of protein kinase. In one embodiment, 103P3E8 antisense oligonucleotides of the present invention are 15 to 30-mer fragments of the antisense DNA molecule that have a sequence that hybridizes to 103P3E8 mRNA. Optionally, 103P3E8 antisense oligonucleotide is a 30-mer oligonucleotide that is complementary to a region in the first 10 5′ codons or last 10 3′ codons of 103P3E8. Alternatively, the antisense molecules are modified to employ ribozymes in the inhibition of 103P3E8 expression, see, e.g., L. A. Couture & D. T. Stinchcomb; Trends Genet 12: 510-515 (1996).

III.A.3.) Primers and Primer Pairs

Further specific embodiments of this nucleotides of the invention include primers and primer pairs, which allow the specific amplification of polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers are used to detect the presence of a 103P3E8 polynucleotide in a sample and as a means for detecting a cell expressing a 103P3E8 protein.

Examples of such probes include polypeptides comprising all or part of the human 103P3E8 cDNA sequence shown in FIG. 2 or FIG. 4. Examples of primer pairs capable of specifically amplifying 103P3E8 mRNAs are also described in the Examples. As will be understood by the skilled artisan, a great many different primers and probes can be prepared based on the sequences provided herein and used effectively to amplify and/or detect a 103P3E8 mRNA.

The 103P3E8 polynucleotides of the invention are useful for a variety of purposes, including but not limited to their use as probes and primers for the amplification and/or detection of the 103P3E8 gene(s), mRNA(s), or fragments thereof; as reagents for the diagnosis and/or prognosis of prostate cancer and other cancers; as coding sequences capable of directing the expression of 103P3E8 polypeptides; as tools for modulating or inhibiting the expression of the 103P3E8 gene(s) and/or translation of the 103P3E8 transcript(s); and as therapeutic agents.

III.A.4.) Isolation of 103P3E8-Encoding Nucleic Acid Molecules

The 103P3E8 cDNA sequences described herein enable the isolation of other polynucleotides encoding 103P3E8 gene product(s), as well as the isolation of polynucleotides encoding 103P3E8 gene product homologs, alternatively spliced isoforms, allelic variants, and mutant forms of the 103P3E8 gene product as well as polynucleotides that encode analogs of 103P3E8-related proteins. Various molecular cloning methods that can be employed to isolate full length cDNAs encoding an 103P3E8 gene are well known (see, for example, Sambrook, J. et al., Molecular Cloning: A Laboratory Manual, 2d edition, Cold Spring Harbor Press, New York, 1989; Current Protocols in Molecular Biology. Ausubel et al., Eds., Wiley and Sons, 1995). For example, lambda phage cloning methodologies can be conveniently employed, using commercially available cloning systems (e.g., Lambda ZAP Express, Stratagene). Phage clones containing 103P3E8 gene cDNAs can be identified by probing with a labeled 103P3E8 cDNA or a fragment thereof. For example, in one embodiment, the 103P3E8 cDNA (FIG. 2 or FIG. 4) or a portion thereof can be synthesized and used as a probe to retrieve overlapping and full-length cDNAs corresponding to a 103P3E8 gene. The 103P3E8 gene itself can be isolated by screening genomic DNA libraries, bacterial artificial chromosome libraries (3ACs), yeast artificial chromosome libraries (YACs), and the like, with 103P3E8 DNA probes or primers.

III.A.5.) Recombinant Nucleic Acid Molecules and Host-Vector Systems

The invention also provides recombinant DNA or RNA molecules containing an 103P3E8 polynucleotide, fragment, analog or homologue thereof, including but not limited to phages, plasmids, phagemids, cosmids, YACs, BACs, as well as various viral and non-viral vectors well known in the art, and cells transformed or transfected with such recombinant DNA or RNA molecules. Methods for generating such molecules are well known (see, for example, Sambrook et al, 1989, supra).

The invention further provides a host-vector system comprising a recombinant DNA molecule containing a 103P3E8 polynucleotide, fragment, analog or homologue thereof within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells). More particularly, a polynucleotide comprising the coding sequence of 103P3E8 or a fragment, analog or homolog thereof can be used to generate 103P3E8 proteins or fragments thereof using any number of host-vector systems routinely used and widely known in the art.

A wide range of host-vector systems suitable for the expression of 103P3E8 proteins or fragments thereof are available, see for example, Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSRαtkneo (Muller et al., 1991, MCB 11:1785). Using these expression vectors, 103P3E8 can be expressed in several prostate cancer and non-prostate cell lines, including for example 293, 293T, rat-1, NIH 3T3 and TsuPr1. The host-vector systems of the invention are useful for the production of a 103P3E8 protein or fragment thereof. Such host-vector systems can be employed to study the functional properties of 103P3E8 and 103P3E8 mutations or analogs.

Recombinant human 103P3E8 protein or an analog or homolog or fragment thereof can be produced by mammalian cells transfected with a construct encoding a 103P3E8-related nucleotide. For example, 293T cells can be transfected with an expression plasmid encoding 103P3E8 or fragment, analog or homolog thereof, the 103P3E8 or related protein is expressed in the 293T cells, and the recombinant 103P3E8 protein is isolated using standard purification methods (e.g., affinity purification using anti-103P3E8 antibodies). In another embodiment, a 103P3E8 coding sequence is subcloned into the retroviral vector pSRαMSVtkneo and used to infect various mammalian cell lines, such as NIH 3T3, TsuPr1, 293 and rat-1 in order to establish 103P3E8 expressing cell lines. Various other expression systems well known in the art can also be employed. Expression constructs encoding a leader peptide joined in frame to the 103P3E8 coding sequence can be used for the generation of a secreted form of recombinant 103P3E8 protein.

As discussed herein, redundancy in the genetic code permits variation in 103P3E8 gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables available on the INTERNET such as: http:H/www.dna.affrc.go.jp/nakamura/codon.html.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell Biol, 9:5073-5080 (1989). Skilled artisans understand that the general rule that eukaryotic ribosomes initiate translation exclusively at the 5′ proximal AUG codon is abrogated only under rare conditions (see, e.g., Kozak PNAS 92(7): 2662-2666, (1995) and Kozak NAR 15(20): 8125-8148 (1987)).

### IV.) 103P3E8-related Proteins

Another aspect of the present invention provides 103P3E8-related proteins. Specific embodiments of 103P3E8 proteins comprise a polypeptide having all or part of the amino acid sequence of human 103P3E8 as shown in FIG. 2, FIG. 3 or FIG. 4. Alternatively, embodiments of 103P3E8 proteins comprise variant, homolog or analog polypeptides that have alterations in the amino acid sequence of 103P3E8 shown in FIG. 2, FIG. 3 or FIG. 4.

In general, naturally occurring allelic variants of human 103P3E8 share a high degree of structural identity and homology (e.g., 90% or more homology). Typically, allelic variants of the 103P3E8 protein contain conservative amino acid substitutions within the 103P3E8 sequences described herein or contain a substitution of an amino acid from a corresponding position in a homologue of 103P3E8. One class of 103P3E8 allelic variants are proteins that share a high degree of homology with at least a small region of a particular 103P3E8 amino acid sequence, but further contain a radical departure from the sequence, such as a non-conservative substitution, truncation, insertion or frame shift. In comparisons of protein sequences, the terms, similarity, identity, and homology each have a distinct meaning as appreciated in the field of genetics. Moreover, orthology and paralogy can be important concepts describing the relationship of members of a given protein family in one organism to the members of the same family in other organisms.

Amino acid abbreviations are provided in Table II. Conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and argimine (R) are frequently interchangeable in locations in which the significant feature of the amino acid residue is its charge and the differing pK's of these two amino acid residues are not significant. Still other changes can be considered “conservative” in particular environments (see, e.g. Table III herein; pages 13-15 “Biochemistry” 2^{nd} ED. Lubert Stryer ed (Stanford University); Henikoff et al., PNAS 1992 Vol 89 10915-10919; Lei et al., J Biol Chem May 19, 1995; 270(20):11882-6).

Embodiments of the invention disclosed herein include a wide variety of art-accepted variants or analogs of 103P3E8 proteins such as polypeptides having amino acid insertions, deletions and substitutions. 103P3E8 variants can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the 103P3E8 variant DNA.

Scanning amino acid analysis can also be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of variant, an isosteric amino acid can be used.

As defined herein, 103P3E8 variants, analogs or homologs, have the distinguishing attribute of having at least one epitope that is “cross reactive” with a 103P3E8 protein having the amino acid sequence of SEQ ID NO: 2. As used in this sentence, “cross reactive” means that an antibody or T cell that specifically binds to an 103P3E8 variant also specifically binds to the 103P3E8 protein having the amino acid sequence of SEQ ID NO: 2. A polypeptide ceases to be a variant of the protein shown in SEQ ID NO: 2 when it no longer contains any epitope capable of being recognized by an antibody or T cell that specifically binds to the 103P3E8 protein. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9):865-73; Schwartz et al., J Immunol (1985) 135(4):2598-608.

Another class of 103P3E8-related protein variants share 70%, 75%, 80%, 85% or 90% or more similarity with the amino acid sequence of SEQ ID NO: 2 or a fragment thereof. Another specific class of 103P3E8 protein variants or analogs comprise one or more of the 103P3E8 biological motifs described herein or presently known in the art. Thus, encompassed by the present invention are analogs of 103P3E8 fragments (nucleic or amino acid) that have altered functional (e.g. immunogenic) properties relative to the starting fragment. It is to be appreciated that motifs now or which become part of the art are to be applied to the nucleic or amino acid sequences of FIG. 2 or FIG. 4.

As discussed herein, embodiments of the claimed invention include polypeptides containing less than the full amino acid sequence of the 103P3E8 protein shown in FIG. 2, FIG. 3, or FIG. 4. For example, representative embodiments of the invention comprise peptides/proteins having any 4, 5, 6, 7, 8, 9, 10, 11, 12, 13, 14, 15 or more contiguous amino acids of the 103P3E8 protein shown in FIG. 2, FIG. 3, or FIG. 4.

Moreover, representative embodiments of the invention disclosed herein include polypeptides consisting of about amino acid 1 to about amino acid 10 of the 103P3E8 protein shown in FIG. 2, FIG. 3 or FIG. 4, polypeptides consisting of about amino acid 10 to about amino acid 20 of the 103P3E8 protein shown in FIG. 2, FIG. 3 or FIG. 4, polypeptides consisting of about amino acid 20 to about amino acid 30 of the 103P3E8 protein shown in FIG. 2, FIG. 3 or FIG. 4, polypeptides consisting of about amino acid 30 to about amino acid 40 of the 103P3E8 protein shown in FIG. 2, FIG. 3 or FIG. 4, polypeptides consisting of about amino acid 40 to about amino acid 50 of the 103P3E8 protein shown in FIG. 2, FIG. 3 or FIG. 4, polypeptides consisting of about amino acid 50 to about amino acid 60 of the 103P3E8 protein shown in FIG. 2, FIG. 3 or FIG. 4, polypeptides consisting of about amino acid 60 to about amino acid 70 of the 103P3E8 protein shown in FIG. 2, FIG. 3 or FIG. 4, polypeptides consisting of about amino acid 70 to about amino acid 80 of the 103P3E8 protein shown in FIG. 2, FIG. 3 or FIG. 4, polypeptides consisting of about amino acid 80 to about amino acid 90 of the 103P3E8 protein shown in FIG. 2, FIG. 3 or FIG. 4, polypeptides consisting of about amino acid 90 to about amino acid 100 of the 103P3E8 protein shown in FIG. 2, FIG. 3 or FIG. 4, etc. throughout the entirety of the 103P3E8 amino acid sequence. Moreover, polypeptides consisting of about amino acid 1 (or 20 or 30 or 40 etc.) to about amino acid 20, (or 130, or 140 or 150 etc.) of the 103P3E8 protein shown in FIG. 2, FIG. 3 or FIG. 4 are embodiments of the invention. It is to be appreciated that the starting and stopping positions in this paragraph refer to the specified position as well as that position plus or minus 5 residues.

103P3E8-related proteins are generated using standard peptide synthesis technology or using chemical cleavage methods well known in the art. Alternatively, recombinant methods can be used to generate nucleic acid molecules that encode a 103P3E8-related protein. In one embodiment, nucleic acid molecules provide a means to generate defined fragments of the 103P3E8 protein (or variants, homologs or analogs thereof).

IV.A.) Motif-bearing Protein Embodiments

Additional illustrative embodiments of the invention disclosed herein include 103P3E8 polypeptides comprising the amino acid residues of one or more of the biological motifs contained within the 103P3E8 polypeptide sequence set forth in FIG. 2, FIG. 3 or FIG. 4. Various motifs are known in the art, and a protein can be evaluated for the presence of such motifs by a number of publicly available sites (see, e.g.: http://pfam.wustl.edu/; http://searchlauncher.bcm.tmc.edu/seq-search/struc-predict.html http://psort.ims.u-tokyo.acjp/; http://www.cbs.dtu.dk/; http://www.ebi.ac.uk/interpro/scan.html; http://www.expasy.ch/tools/scnpsitl.html; Epiratrix™ and Epimer™, Brown University, http://www.brown.edu/Research/TB-HIV Lab/epimatrix/epimatrix.html; and BIMAS, http://bimas.dcrt.nih.gov/.). Motif bearing subsequences of the 103P3E8 protein are set forth and identified in Table XIX.

Table XX sets forth several frequently occurring motifs based on pfam searches (http://pfam.wustl.edu/). The columns of Table XX list (1) motif name abbreviation, (2) percent identity found amongst the different member of the motif family, (3) motif name or description and (4) most common function; location information is included if the motif is relevant for location.

Polypeptides comprising one or more of the 103P3E8 motifs discussed above are useful in elucidating the specific characteristics of a malignant phenotype in view of the observation that the 103P3E8 motifs discussed above are associated with growth dysregulation and because 103P3E8 is overexpressed in certain cancers (See, e.g., Table I). Casein kinase II, cAMP and camp-dependent protein kinase, and Protein Kinase C, for example, are enzymes known to be associated with the development of the malignant phenotype (see e.g. Chen et al., Lab Invest., 78(2): 165-174 (1998); Gaiddon et al., Endocrinology 136(10): 4331-4338 (1995); Hall et al., Nucleic Acids Research 24(6): 1119-1126 (1996); Peterziel et al., Oncogene 18(46): 6322-6329 (1999) and O'Brian, Oncol. Rep. 5(2): 305-309 (1998)). Moreover, both glycosylation and myristoylation are protein modifications also associated with cancer and cancer progression (see e.g. Dennis et al., Biochem. Biophys. Acta 1473(1):21-34 (1999); Raju et al., Exp. Cell Res. 235(1): 145-154 (1997)). Amidation is another protein modification also associated with cancer and cancer progression (see e.g. Treston et al., J. Natl. Cancer Inst. Monogr. (13): 169-175 (1992)).

In another embodiment, proteins of the invention comprise one or more of the immunoreactive epitopes identified in accordance with art-accepted methods, such as the peptides set forth in Tables V-XVIII. CTL epitopes can be determined using specific algorithms to identify peptides within an 103P3E8 protein that are capable of optimally binding to specified HLA alleles (e.g., Table IV (A) and Table IV (13); Epimatrix™ and Epimer™, Brown University, http://www.brown.edu/Research/TB-HIV_Lab/epimatrix/epimatrix.html; and BIMAS, http://bimas.dcrt.nih.gov/. Moreover, processes for identifying peptides that have sufficient binding affinity for HLA molecules and which are correlated with being immunogenic epitopes, are well known in the art, and are carried out without undue experimentation. In addition, processes for identifying peptides that are immunogenic epitopes, are well known in the art, and are carried out without undue experimentation either in vitro or in vivo.

Also known in the art are principles for creating analogs of such epitopes in order to modulate immunogenicity. For example, one begins with an epitope that bears a CTL or HTL motif (see, e.g., the HLA Class I motifs or Table IV (A) and the HTL motif of Table IV (B)). The epitope is analoged by substituting out an amino acid at one of the specified positions, and replacing it with another amino acid specified for that position.

A variety of references reflect the art regarding the identification and generation of epitopes in a protein of interest as well as analogs thereof. See, for example, WO 9733602 to Chesnut et al.; Sette, Immunogenetics 1999 50(3-4): 201-212; Sette et al., J. Immunol. 2001 166(2): 1389-1397; Sidney et al., Hum. Immunol. 1997 58(1): 12-20; Kondo et al., Immunogenetics 1997 45(4): 249-258; Sidney et al., J. Immunol. 1996 157(8): 3480-90; and Falk et al., Nature 351: 290-6 (1991); Hunt et al., Science 255:1261-3 (1992); Parker et al., J. Immunol. 149:3580-7 (1992); Parker et al., J. Immunol. 152:163-75 (1994)); Kast et al., 1994 152(8): 3904-12; Borras-Cuesta et al., Hum. Immunol. 2000 61(3): 266-278; Alexander et al., J. Immunol. 2000 164(3); 164(3): 1625-1633; Alexander et al., PMID: 7895164, UI: 95202582; O'Sullivan et al., J. Immunol. 1991 147(8): 2663-2669; Alexander et al., Immunity 1994 1(9): 751-761 and Alexander et al., Immunol. Res. 1998 18(2): 79-92.

Related embodiments of the inventions include polypeptides comprising combinations of the different motifs set forth in Table XIX, and/or, one or more of the predicted CTL epitopes of Table V through Table XVIII, and/or, one or more of the T cell binding motifs known in the art. Preferred embodiments contain no insertions, deletions or substitutions either within the motifs or the intervening sequences of the polypeptides. In addition, embodiments which include a number of either N-terminal and/or C-terminal amino acid residues on either side of these motifs may be desirable (to, for example, include a greater portion of the polypeptide architecture in which the motif is located). Typically the number of N-terminal and/or C-terminal amino acid residues on either side of a motif is between about 1 to about 100 amino acid residues, preferably 5 to about 50 amino acid residues.

103P3E8-related proteins are embodied in many forms, preferably in isolated form. A purified 103P3E8 protein molecule will be substantially free of other proteins or molecules that impair the binding of 103P3E8 to antibody, T cell or other ligand. The nature and degree of isolation and purification will depend on the intended use. Embodiments of a 103P3E8-related proteins include purified 103P3E8-related proteins and functional, soluble 103P3E8-related proteins. In one embodiment, a functional, soluble 103P3E8 protein or fragment thereof retains the ability to be bound by antibody, T cell or other ligand.

The invention also provides 103P3E8 proteins comprising biologically active fragments of the 103P3E8 amino acid sequence shown in FIG. 2, FIG. 3 or FIG. 4. Such proteins exhibit properties of the 103P3E8 protein, such as the ability to elicit the generation of antibodies that specifically bind an epitope associated with the 103P3E8 protein; to be bound by such antibodies; to elicit the activation of HTL or CTL; and/or, to be recognized by HTL or CTL.

103P3E8-related polypeptides that contain particularly interesting structures can be predicted and/or identified using various analytical techniques well known in the art, including, for example, the methods of Chou-Fasman, Garnier-Robson, Kyte-Doolittle, Eisenberg, Karplus-Schultz or Jameson-Wolf analysis, or on the basis of immunogenicity. Fragments that contain such structures are particularly useful in generating subunit-specific anti-103P3E8 antibodies, or T cells or in identifying cellular factors that bind to 103P3E8.

CTL epitopes can be determined using specific algorithms to identify peptides within an 103P3E8 protein that are capable of optimally binding to specified HLA alleles (e.g., Table IV (A) and Table IV (B); Epimatrix™ and Epimer™, Brown University (http://www.brown.edu/Research/TB-HIV_Lab/epimatrix/epimatrix.html); and BIMAS, http://bimas.dcrt.nih.gov/). Illustrating this, peptide epitopes from 103P3E8 that are presented in the context of human MHC class I molecules HLA-A1, A2, A3, All, A24, B7 and B35 were predicted (Tables V-XVIII). Specifically, the complete amino acid sequence of the 103P3E8 protein was entered into the HLA Peptide Motif Search algorithm found in the Bioinformatics and Molecular Analysis Section (BIMAS) web site listed above. The HLA peptide motif search algorithm was developed by Dr. Ken Parker based on binding of specific peptide sequences in the groove of HLA Class I molecules and specifically HLA-A2 (see, e.g., Falk et al., Nature 351: 290-6 (1991); Hunt et al., Science 255:1261-3 (1992); Parker et al., J. Immunol. 149:3580-7 (1992); Parker et al., J. Immunol. 152:163-75 (1994)). This algorithm allows location and ranking of 8-mer, 9-mer, and 10-mer peptides from a complete protein sequence for predicted binding to HLA-A2 as well as numerous other HLA Class I molecules. Many HLA class I binding peptides are 8-, 9-, 10 or 11-mers. For example, for class I HLA-A2, the epitopes preferably contain a leucine (L) or methionine (M) at position 2 and a valine (V) or leucine (L) at the C-terminus (see, e.g., Parker et al., J. Immunol. 149:3580-7 (1992)). Selected results of 103P3E8 predicted binding peptides are shown in Tables V-XVIII herein. In Tables V-XVIII, the top 50 ranking candidates, 9-mers and 10-mers, for each family member are shown along with their location, the amino acid sequence of each specific peptide, and an estimated binding score. The binding score corresponds to the estimated half time of dissociation of complexes containing the peptide at 37° C. at pH 6.5. Peptides with the highest binding score are predicted to be the most tightly bound to HLA Class I on the cell surface for the greatest period of time and thus represent the best immunogenic targets for T-cell recognition.

Actual binding of peptides to an HLA allele can be evaluated by stabilization of HLA expression on the antigen-processing defective cell line T2 (see, e.g., Xue et al., Prostate 30:73-8 (1997) and Peshwa et al., Prostate 36:129-38 (1998)). Immunogenicity of specific peptides can be evaluated in vitro by stimulation of CD8+ cytotoxic T lymphocytes (CTL) in the presence of antigen presenting cells such as dendritic cells.

It is to be appreciated that every epitope predicted by the BIMAS site, Epimer™ and Epimatrix™ sites, or specified by the HLA class I or class I motifs available in the art or which become part of the art such as set forth in Table IV (A) and Table IV (B) are to be “applied” to the 103P3E8 protein. As used in this context “applied” means that the 103P3E8 protein is evaluated, e.g., visually or by computer-based patterns finding methods, as appreciated by those of skill in the relevant art. Every subsequence of the 103P3E8 of 8, 9, 10, or 11 amino acid residues that bears an HLA Class I motif, or a subsequence of 9 or more amino acid residues that bear an HLA Class II motif are within the scope of the invention.

IV.B.) Expression of 103P3E8-related Proteins

In an embodiment described in the examples that follow, 103P3E8 can be conveniently expressed in cells (such as 293T cells) transfected with a commercially available expression vector such as a CMV-driven expression vector encoding 103P3E8 with a C-terminal 6XHis and MYC tag (pcDNA3.1/mycHIS, Invitrogen or Tag5, GenHunter Corporation, Nashville Tenn.). The Tag5 vector provides an IgGK secretion signal that can be used to facilitate the production of a secreted 103P3E8 protein in transfected cells. The secreted HIS-tagged 103P3E8 in the culture media can be purified, e.g., using a nickel column using standard techniques.

IV.C.) Modifications of 103P3E8-related Proteins

Modifications of 103P3E8-related proteins such as covalent modifications are included within the scope of this invention. One type of covalent modification includes reacting targeted amino acid 30 residues of a 103P3E8 polypeptide with an organic derivatizing agent that is capable of reacting with selected side chains or the N- or C- terminal residues of the 103P3E8. Another type of covalent modification of the 103P3E8 polypeptide included within the scope of this invention comprises altering the native glycosylation pattern of a protein of the invention. Another type of covalent modification of 103P3E8 comprises linking the 103P3E8 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The 103P3E8-related proteins of the present invention can also be modified to form a chimeric molecule comprising 103P3E8 fused to another, heterologous polypeptide or amino acid sequence. Such a chimeric molecule can be synthesized chemically or recombinantly. A chimeric molecule can have a protein of the invention fused to another tumor-associated antigen or fragment thereof. Alternatively, a protein in accordance with the invention can comprise a fusion of fragments of the 103P3E8 sequence (amino or nucleic acid) such that a molecule is created that is not, through its length, directly homologous to the amino or nucleic acid sequences respectively of FIG. 2 or FIG. 4. Such a chimeric molecule can comprise multiples of the same subsequence of 103P3E8. A chimeric molecule can comprise a fusion of a 103P3E8-related protein with a polyhistidine epitope tag, which provides an epitope to which immobilized nickel can selectively bind, with cytokines or with growth factors. The epitope tag is generally placed at the amino- or carboxyl- terminus of the 103P3E8. In an alternative embodiment, the chimeric molecule can comprise a fusion of a 103P3E8-related protein with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a 103P3E8 polypeptide in place of at least one variable region within an Ig molecule. In a preferred embodiment, the immunoglobulin fusion includes the hinge, CH2 and CH3, or the hinge, CH1, CH2 and CH3 regions of an IgGI molecule. For the production of immunoglobulin fusions see, e.g., U.S. Pat. No. 5,428,130 issued Jun. 27, 1995.

IV.D.) Uses of 103P3E8-related Proteins

The proteins of the invention have a number of different specific uses. As 103P3E8 is highly expressed in prostate and other cancers, 103P3E8-related proteins are used in methods that assess the status of 103P3E8 gene products in normal versus cancerous tissues, thereby elucidating the malignant phenotype. Typically, polypeptides from specific regions of the 103P3E8 protein are used to assess the presence of perturbations (such as deletions, insertions, point mutations etc.) in those regions (such as regions containing one or more motifs). Exemplary assays utilize antibodies or T cells targeting 103P3E8-related proteins comprising the amino acid residues of one or more of the biological motifs contained within the 103P3E8 polypeptide sequence in order to evaluate the characteristics of this region in normal versus cancerous tissues or to elicit an immune response to the epitope. Alternatively, 103P3E8-related proteins that contain the amino acid residues of one or more of the biological motifs in the 103P3E8 protein are used to screen for factors that interact with that region of 103P3E8.

103P3E8 protein fragments/subsequences are particularly useful in generating and characterizing domain-specific antibodies (e.g., antibodies recognizing an extracellular or intracellular epitope of an 103P3E8 protein), for identifying agents or cellular factors that bind to 103P3E8 or a particular structural domain thereof, and in various therapeutic and diagnostic contexts, including but not limited to diagnostic assays, cancer vaccines and methods of preparing such vaccines.

Proteins encoded by the 103P3E8 genes, or by analogs, homologs or fragments thereof, have a variety of uses, including but not limited to generating antibodies and in methods for identifying ligands and other agents and cellular constituents that bind to an 103P3E8 gene product. Antibodies raised against an 103P3E8 protein or fragment thereof are useful in diagnostic and prognostic assays, and imaging methodologies in the management of human cancers characterized by expression of 103P3E8 protein, such as those listed in Table I. Such antibodies can be expressed intracellularly and used in methods of treating patients with such cancers. 103P3E8-related nucleic acids or proteins are also used in generating HTL or CTL responses.

Various immunological assays useful for the detection of 103P3E8 proteins are used, including but not limited to various types of radioimmunoassays, enzyme-linked immunosorbent assays (ELISA), enzyme-linked immunofluorescent assays (ELIFA), immunocytochemical methods, and the like. Antibodies can be labeled and used as immunological imaging reagents capable of detecting 103P3E8-expressing cells (e.g., in radioscintigraphic imaging methods). 103P3E8 proteins are also particularly useful in generating cancer vaccines, as further described herein.

### V.) 103P3E8 Antibodies

Another aspect of the invention provides antibodies that bind to 103P3E8-related proteins. Preferred antibodies specifically bind to a 103P3E8-related protein and do not bind (or bind weakly) to peptides or proteins that are not 103P3E8-related proteins. For example, antibodies bind 103P3E8 can bind 103P3E8-related proteins such as the homologs or analogs thereof.

103P3E8 antibodies of the invention are particularly useful in prostate cancer diagnostic and prognostic assays, and imaging methodologies. Similarly, such antibodies are useful in the treatment, diagnosis, and/or prognosis of other cancers, to the extent 103P3E8 is also expressed or overexpressed in these other cancers. Moreover, intracellularly expressed antibodies (e.g., single chain antibodies) are therapeutically useful in treating cancers in which the expression of 103P3E8 is involved, such as advanced or metastatic prostate cancers.

The invention also provides various immunological assays useful for the detection and quantification of 103P3E8 and mutant 103P3E8-related proteins. Such assays can comprise one or more 103P3E8 antibodies capable of recognizing and binding a 103P3E8-related protein, as appropriate. These assays are performed within various immunological assay formats well known in the art, including but not limited to various types of radioimmunoassays, enzyme-linked immunosorbent assays (ELISA), enzyme-linked immunofluorescent assays (ELIFA), and the like.

Immunological non-antibody assays of the invention also comprise T cell immunogenicity assays (inhibitory or stimulatory) as well as major histocompatibility complex (MHC) binding assays.

In addition, immunological imaging methods capable of detecting prostate cancer and other cancers expressing 103P3E8 are also provided by the invention, including but not limited to radioscintigraphic imaging methods using labeled 103P3E8 antibodies. Such assays are clinically useful in the detection, monitoring, and prognosis of 103P3E8 expressing cancers such as prostate cancer.

103P3E8 antibodies are also used in methods for purifying a 103P3E8-related protein and for isolating 103P3E8 homologues and related molecules. For example, a method of purifying a 103P3E8-related protein comprises incubating an 103P3E8 antibody, which has been coupled to a solid matrix, with a lysate or other solution containing a 103P3E8-related protein under conditions that permit the 103P3E8 antibody to bind to the 103P3E8-related protein; washing the solid matrix to eliminate impurities; and eluting the 103P3E8-related protein from the coupled antibody. Other uses of the 103P3E8 antibodies of the invention include generating anti-idiotypic antibodies that mimic the 103P3E8 protein.

Various methods for the preparation of antibodies are well known in the art. For example, antibodies can be prepared by immunizing a suitable mammalian host using a 103P3E8-related protein, peptide, or fragment, in isolated or immunoconjugated form (Antibodies: A Laboratory Manual, CSH Press, Eds., Harlow, and Lane (1988); Harlow, Antibodies, Cold Spring Harbor Press, N.Y. (1989)). In addition, fusion proteins of 103P3E8 can also be used, such as a 103P3E8 GST-fusion protein. In a particular embodiment, a GST fusion protein comprising all or most of the amino acid sequence of FIG. 2, FIG. 3 or FIG. 4 is produced, then used as an immunogen to generate appropriate antibodies. In another embodiment, a 103P3E8-related protein is synthesized and used as an immunogen.

In addition, naked DNA immunization techniques known in the art are used (with or without purified 103P3E8-related protein or 103P3E8 expressing cells) to generate an immune response to the encoded immunogen (for review, see Donnelly et al., 1997, Ann. Rev. Immunol. 15: 617-648).

The amino acid sequence of 103P3E8 as shown in FIG. 2, FIG. 3 or FIG. 4 can be analyzed to select specific regions of the 103P3E8 protein for generating antibodies. For example, hydrophobicity and hydrophilicity analyses of the 103P3E8 amino acid sequence are used to identify hydrophilic regions in the 103P3E8 structure. Regions of the 103P3E8 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Thus, each region identified by any of these programs or methods is within the scope of the present invention. Methods for the generation of 103P3E8 antibodies are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents such as those supplied by Pierce Chemical Co., Rockford, Ill., are effective. Administration of a 103P3E8 immunogen is often conducted by injection over a suitable time period and with use of a suitable adjuvant, as is understood in the art. During the immunization schedule, titers of antibodies can be taken to determine adequacy of antibody formation.

103P3E8 monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a 103P3E8-related protein. When the appropriate immortalized cell culture is identified, the cells can be expanded and antibodies produced either from in vitro cultures or from ascites fluid.

The antibodies or fragments of the invention can also be produced, by recombinant means. Regions that bind specifically to the desired regions of the 103P3E8 protein can also be produced in the context of chimeric or complementarity determining region (CDR) grafted antibodies of multiple species origin. Humanized or human 103P3E8 antibodies can also be produced, and are preferred for use in therapeutic contexts. Methods for humanizing murine and other non-human antibodies, by substituting one or more of the non-human antibody CDRs for corresponding human antibody sequences, are well known (see for example, Jones et al., 1986, Nature 321: 522-525; Riechmann et al., 1988, Nature 332: 323-327; Verhoeyen et al., 1988, Science 239: 1534-1536). See also, Carter et al., 1993, Proc. Natl. Acad. Sci. USA 89: 4285 and Sims et al., 1993, J. Immunol. 151: 2296.

Methods for producing fully human monoclonal antibodies include phage display and transgenic methods (for review, see Vaughan et al., 1998, Nature Biotechnology 16: 535-539). Fully human 103P3E8 monoclonal antibodies can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man, Clark, M. (Ed.), Nottingham Academic, pp 45-64 (1993); Burton and Barbas, Human Antibodies from combinatorial libraries. Id., pp 65-82). Fully human 103P3E8 monoclonal antibodies can also be produced using transgenic mice engineered to contain human immunoglobulin gene loci as described in PCT Patent Application WO98/24893, Kucherlapati and Jakobovits et al., published Dec. 3, 1997 (see also, Jakobovits, 1998, Exp. Opin. Invest. Drugs 7(4): 607-614; U.S. Pat. Nos. 6,162,963 issued Dec. 19, 2000; 6,150,584 issued Nov. 12, 2000; and, 6,114598 issued Sep. 5, 2000). This method avoids the in vitro manipulation required with phage display technology and efficiently produces high affinity authentic human antibodies.

Reactivity of 103P3E8 antibodies with an 103P3E8-related protein can be established by a number of well known means, including Western blot, immunoprecipitation, ELISA, and FACS analyses using, as appropriate, 103P3E8-related proteins, 103P3E8-expressing cells or extracts thereof. A 103P3E8 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 103P3E8 epitopes are generated using methods generally known in the art. Homodimeric antibodies can also be generated by cross-linking techniques known in the art (e.g., Wolff et al., Cancer Res. 53: 2560-2565).

### VI.) 103P3E8 Transgenic Animals

Nucleic acids that encode a 103P3E8-related protein can also be used to generate either transgenic animals or “knock out” animals which, in turn, are useful in the development and screening of therapeutically useful reagents. In accordance with established techniques, cDNA encoding 103P3E8 can be used to clone genomic DNA that encodes 103P3E8. The cloned genomic sequences can then be used to generate transgenic animals containing cells that express DNA that encode 103P3E8. Methods for generating transgenic animals, particularly animals such as mice or rats, have become conventional in the art and are described, for example, in U.S. Pat. Nos. 4,736,866 issued Apr. 12, 1988, and 4,870,009 issued Sep. 26, 1989. Typically, particular cells would be targeted for 103P3E8 transgene incorporation with tissue-specific enhancers.

Transgenic animals that include a copy of a transgene encoding 103P3E8 can be used to examine the effect of increased expression of DNA that encodes 103P3E8. Such animals can be used as tester animals for reagents thought to confer protection from, for example, pathological conditions associated with its overexpression. In accordance with this aspect of the invention, an animal is treated with a reagent and a reduced incidence of a pathological condition, compared to untreated animals that bear the transgene, would indicate a potential therapeutic intervention for the pathological condition.

Alternatively, non-human homologues of 103P3E8 can be used to construct a 103P3E8 “knock out” animal that has a defective or altered gene encoding 103P3E8 as a result of homologous recombination between the endogenous gene encoding 103P3E8 and altered genomic DNA encoding 103P3E8 introduced into an embryonic cell of the animal. For example, cDNA that encodes 103P3E8 can be used to clone genomic DNA encoding 103P3E8 in accordance with established techniques. A portion of the genomic DNA encoding 103P3E8 can be deleted or replaced with another gene, such as a gene encoding a selectable marker that can be used to monitor integration. Typically, several kilobases of unaltered flanking DNA (both at the 5′ and 3′ ends) are included in the vector (see, e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (see, e.g., Li et al., Cell, 69:915 (1992)). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal, and the embryo brought to term to create a “knock out” animal. Progeny harboring the homologously recombined DNA in their germ cells can be identified by standard techniques and used to breed animals in which all cells of the animal contain the homologously recombined DNA. Knock out animals can be characterized, for example, for their ability to defend against certain pathological conditions or for their development of pathological conditions due to absence of the 103P3E8 polypeptide.

### VII.) Methods for the Detection of 103P3E8

Another aspect of the present invention relates to methods for detecting 103P3E8 polynucleotides and 103P3E8-related proteins, as well as methods for identifying a cell that expresses 103P3E8. The expression profile of 103P3E8 makes it a diagnostic marker for metastasized disease. Accordingly, the status of 103P3E8 gene products provides information useful for predicting a variety of factors including susceptibility to advanced stage disease, rate of progression, and/or tumor aggressiveness. As discussed in detail herein, the status of 103P3E8 gene products in patient samples can be analyzed by a variety protocols that are well known in the art including immunohistochemical analysis, the variety of Northern blotting techniques including in situ hybridization, RT-PCR analysis (for example on laser capture micro-dissected samples), Western blot analysis and tissue array analysis.

More particularly, the invention provides assays for the detection of 103P3E8 polynucleotides in a biological sample, such as serum, bone, prostate, and other tissues, urine, semen, cell preparations, and the like. Detectable 103P3E8 polynucleotides include, for example, a 103P3E8 gene or fragment thereof, 103P3E8 mRNA, alternative splice variant 103P3E8 mRNAs, and recombinant DNA or RNA molecules that contain a 103P3E8 polynucleotide. A number of methods for amplifying and/or detecting the presence of 103P3E8 polynucleotides are well known in the art and can be employed in the practice of this aspect of the invention.

In one embodiment, a method for detecting an 103P3E8 MRNA in a biological sample comprises producing cDNA from the sample by reverse transcription using at least one primer; amplifying the cDNA so produced using an 103P3E8 polynucleotides as sense and antisense primers to amplify 103P3E8 cDNAs therein; and detecting the presence of the amplified 103P3E8 cDNA. Optionally, the sequence of the amplified 103P3E8 cDNA can be determined.

In another embodiment, a method of detecting a 103P3E8 gene in a biological sample comprises first isolating genomic DNA from the sample; amplifying the isolated genomic DNA using 103P3E8 polynucleotides as sense and antisense primers; and detecting the presence of the amplified 103P3E8 gene. Any number of appropriate sense and antisense probe combinations can be designed from the nucleotide sequence provided for the 103P3E8 (FIG. 2 or FIG. 4) and used for this purpose.

The invention also provides assays for detecting the presence of an 103P3E8 protein in a tissue or other biological sample such as serum, semen, bone, prostate, urine, cell preparations, and the like. Methods for detecting a 103P3E8-related protein are also well known and include, for example, immunoprecipitation, immunohistochemical analysis, Western blot analysis, molecular binding assays, ELISA, ELIFA and the like. For example, a method of detecting the presence of a 103P3E8-related protein in a biological sample comprises first contacting the sample with a 103P3E8 antibody, a 103P3E8-reactive fragment thereof, or a recombinant protein containing an antigen binding region of a 103P3E8 antibody; and then detecting the binding of 103P3E8-related protein in the sample.

Methods for identifying a cell that expresses 103P3E8 are also within the scope of the invention. In one embodiment, an assay for identifying a cell that expresses a 103P3E8 gene comprises detecting the presence of 103P3E8 mRNA in the cell. Methods for the detection of particular mRNAs in cells are well known and include, for example, hybridization assays using complementary DNA probes (such as in situ hybridization using labeled 103P3E8 riboprobes, Northern blot and related techniques) and various nucleic acid amplification assays (such as RT-PCR using complementary primers specific for 103P3E8, and other amplification type detection methods, such as, for example, branched DNA, SISBA, TMA and the like). Alternatively, an assay for identifying a cell that expresses a 103P3E8 gene comprises detecting the presence of 103P3E8-related protein in the cell or secreted by the cell. Various methods for the detection of proteins are well known in the art and are employed for the detection of 103P3E8-related proteins and cells that express 103P3E8-related proteins.

103P3E8 expression analysis is also useful as a tool for identifying and evaluating agents that modulate 103P3E8 gene expression. For example, 103P3E8 expression is significantly upregulated in prostate cancer, and is expressed in cancers of the tissues listed in Table I. Identification of a molecule or biological agent that inhibits 103P3E8 expression or over-expression in cancer cells is of therapeutic value. For example, such an agent can be identified by using a screen that quantifies 103P3E8 expression by RT-PCR, nucleic acid hybridization or antibody binding.

### VIII.) Methods for Monitoring the Status of 103P3E8-related Genes and Their Products

Oncogenesis is known to be a multistep process where cellular growth becomes progressively dysregulated and cells progress from a normal physiological state to precancerous and then cancerous states (see, e.g., Alers et al., Lab Invest. 77(5): 437-438 (1997) and Isaacs et al., Cancer Surv. 23: 19-32 (1995)). In this context, examining a biological sample for evidence of dysregulated cell growth (such as aberrant 103P3E8 expression in cancers) allows for early detection of such aberrant physiology, before a pathologic state such as cancer has progressed to a stage that therapeutic options are more limited and or the prognosis is worse. In such examinations, the status of 103P3E8 in a biological sample of interest can be compared, for example, to the status of 103P3E8 in a corresponding normal sample (e.g. a sample from that individual or alternatively another individual that is not affected by a pathology). An alteration in the status of 103P3E8 in the biological sample (as compared to the normal sample) provides evidence of dysregulated cellular growth. In addition to using a biological sample that is not affected by a pathology as a normal sample, one can also use a predetermined normative value such as a predetermined normal level of mRNA expression (see, e.g., Grever et al., J. Comp. Neurol. Dec. 9, 1996;376(2):306-14 and U.S. Pat. No. 5,837,501) to compare 103P3E8 status in a sample.

The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of 103P3E8 expressing cells) as well as the level, and biological activity of expressed gene products (such as 103P3E8 mRNA, polynucleotides and polypeptides). Typically, an alteration in the status of 103P3E8 comprises a change in the location of 103P3E8 and/or 103P3E8 expressing cells and/or an increase in 103P3E8 mRNA and/or protein expression.

103P3E8 status in a sample can be analyzed by a number of means well known in the art, including without limitation, immunohistochemical analysis, in situ hybridization, RT-PCR analysis on laser capture micro-dissected samples, Western blot analysis, and tissue array analysis. Typical protocols for evaluating the status of the 103P3E8 gene and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Thus, the status of 103P3E8 in a biological sample is evaluated by various methods utilized by skilled artisans including, but not limited to genomic Southern analysis (to examine, for example perturbations in the 103P3E8 gene), Northern analysis and/or PCR analysis of 103P3E8 mRNA (to examine, for example alterations in the polynucleotide sequences or expression levels of 103P3E8 mRNAs), and, Western and/or immunohistochemical analysis (to examine, for example alterations in polypeptide sequences, alterations in polypeptide localization within a sample, alterations in expression levels of 103P3E8 proteins and/or associations of 103P3E8 proteins with polypeptide binding partners). Detectable 103P3E8 polynucleotides include, for example, a 103P3E8 gene or fragment thereof, 103P3E8 mRNA, alternative splice variants, 103P3E8 mRNAs, and recombinant DNA or RNA molecules containing a 103P3E8 polynucleotide.

The expression profile of 103P3E8 makes it a diagnostic marker for local and/or metastasized disease, and provides information on the growth or oncogenic potential of a biological sample. In particular, the status of 103P3E8 provides information useful for predicting susceptibility to particular disease stages, progression, and/or tumor aggressiveness. The invention provides methods and assays for determining 103P3E8 status and diagnosing cancers that express 103P3E8, such as cancers of the tissues listed in Table I. For example, because 103P3E8 mRNA is so highly expressed in prostate and other cancers relative to normal prostate tissue, assays that evaluate the levels of 103P3E8 mRNA transcripts or proteins in a biological sample can be used to diagnose a disease associated with 103P3E8 dysregulation, and can provide prognostic information useful in defining appropriate therapeutic options.

The expression status of 103P3E8 provides information including the presence, stage and location of dysplastic, precancerous and cancerous cells, predicting susceptibility to various stages of disease, and/or for gauging tumor aggressiveness. Moreover, the expression profile makes it useful as an imaging reagent for metastasized disease. Consequently, an aspect of the invention is directed to the various molecular prognostic and diagnostic methods for examining the status of 103P3E8 in biological samples such as those from individuals suffering from, or suspected of suffering from a pathology characterized by dysregulated cellular growth, such as cancer.

As described above, the status of 103P3E8 in a biological sample can be examined by a number of well-known procedures in the art. For example, the status of 103P3E8 in a biological sample taken from a specific location in the body can be examined by evaluating the sample for the presence or absence of 103P3E8 expressing cells (e.g. those that express 103P3E8 mRNAs or proteins). This examination can provide evidence of dysregulated cellular growth, for example, when 103P3E8-expressing cells are found in a biological sample that does not normally contain such cells (such as a lymph node), because such alterations in the status of 103P3E8 in a biological sample are often associated with dysregulated cellular growth. Specifically, one indicator of dysregulated cellular growth is the metastases of cancer cells from an organ of origin (such as the prostate) to a different area of the body (such as a lymph node). In this context, evidence of dysregulated cellular growth is important for example because occult lymph node metastases can be detected in a substantial proportion of patients with prostate cancer, and such metastases are associated with known predictors of disease progression (see, e.g., Murphy et al., Prostate 42(4): 315-317 (2000);Su et al., Semin. Surg. Oncol. 18(1): 17-28 (2000) and Freeman et al., J Urol 1995 Aug 154(2 Pt 1):474-8).

In one aspect, the invention provides methods for monitoring 103P3E8 gene products by determining the status of 103P3E8 gene products expressed by cells from an individual suspected of having a disease associated with dysregulated cell growth (such as hyperplasia or cancer) and then comparing the status so determined to the status of 103P3E8 gene products in a corresponding normal sample. The presence of aberrant 103P3E8 gene products in the test sample relative to the normal sample provides an indication of the presence of dysregulated cell growth within the cells of the individual.

In another aspect, the invention provides assays useful in determining the presence of cancer in an individual, comprising detecting a significant increase in 103P3E8 mRNA or protein expression in a test cell or tissue sample relative to expression levels in the corresponding normal cell or tissue. The presence of 103P3E8 mRNA can, for example, be evaluated in tissue samples including but not limited to those listed in Table I. The presence of significant 103P3E8 expression in any of these tissues is useful to indicate the emergence, presence and/or severity of a cancer, since the corresponding normal tissues do not express 103P3E8 mRNA or express it at lower levels.

In a related embodiment, 103P3E8 status is determined at the protein level rather than at the nucleic acid level. For example, such a method comprises determining the level of 103P3E8 protein expressed by cells in a test tissue sample and comparing the level so determined to the level of 103P3E8 expressed in a corresponding normal sample. In one embodiment, the presence of 103P3E8 protein is evaluated, for example, using immunohistochemical methods. 103P3E8 antibodies or binding partners capable of detecting 103P3E8 protein expression are used in a variety of assay formats well known in the art for this purpose.

In a further embodiment, one can evaluate the status of 103P3E8 nucleotide and amino acid sequences in a biological sample in order to identify perturbations in the structure of these molecules. These perturbations can include insertions, deletions, substitutions and the like. Such evaluations are useful because perturbations in the nucleotide and amino acid sequences are observed in a large number of proteins associated with a growth dysregulated phenotype (see, e.g., Marrogi et al., 1999, J. Cutan. Pathol. 26(8):369-378). For example, a mutation in the sequence of 103P3E8 may be indicative of the presence or promotion of a tumor. Such assays therefore have diagnostic and predictive value where a mutation in 103P3E8 indicates a potential loss of function or increase in tumor growth.

A wide variety of assays for observing perturbations in nucleotide and amino acid sequences are well known in the art. For example, the size and structure of nucleic acid or amino acid sequences of 103P3E8 gene products are observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 issued Sep. 7, 1999, and 5,952,170 issued Jan. 17, 1995).

Additionally, one can examine the methylation status of the 103P3E8 gene in a biological sample. Aberrant demethylation and/or hypermethylation of CpG islands in gene 5′ regulatory regions frequently occurs in immortalized and transformed cells, and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6): 1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6): 903-908 (1998)). A variety of assays for examining methylation status of a gene are well known in the art. For example, one can utilize, in Southern hybridization approaches, methylation-sensitive restriction enzymes which cannot cleave sequences that contain methylated CpG sites to assess the methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubel et al. eds., 1995.

Gene amplification is an additional method for assessing the status of 103P3E8. Gene amplification is measured in a sample directly, for example, by conventional Southern blotting or Northern blotting to quantitate the transcription of mRNA (Thomas, 1980, Proc. Natl. Acad. Sci. USA, 77:5201-5205), dot blotting (DNA analysis), or in situ hybridization, using an appropriately labeled probe, based on the sequences provided herein. Alternatively, antibodies are employed that recognize specific duplexes, including DNA duplexes, RNA duplexes, and DNA-RNA hybrid duplexes or DNA-protein duplexes. The antibodies in turn are labeled and the assay carried out where the duplex is bound to a surface, so that upon the formation of duplex on the surface, the presence of antibody bound to the duplex can be detected.

Biopsied tissue or peripheral blood can be conveniently assayed for the presence of cancer cells using for example, Northern, dot blot or RT-PCR analysis to detect 103P3E8 expression. The presence of RT-PCR amplifiable 103P3E8 mRNA provides an indication of the presence of cancer. RT-PCR assays are well known in the art. RT-PCR detection assays for tumor cells in peripheral blood are currently being evaluated for use in the diagnosis and management of a number of human solid tumors. In the prostate cancer field, these include RT-PCR assays for the detection of cells expressing PSA and PSM (Verkaik et al., 1997, Urol. Res. 25:373-384; Ghossein et al., 1995, J. Clin. Oncol. 13:1195-2000; Heston et al., 1995, Clin. Chem. 41:1687-1688).

A further aspect of the invention is an assessment of the susceptibility that an individual has for developing cancer. In one embodiment, a method for predicting susceptibility to cancer comprises detecting 103P3E8 mRNA or 103P3E8 protein in a tissue sample, its presence indicating susceptibility to cancer, wherein the degree of 103P3E8 mRNA expression correlates to the degree of susceptibility. In a specific embodiment, the presence of 103P3E8 in prostate or other tissue is examined, with the presence of 103P3E8 in the sample providing an indication of prostate cancer susceptibility (or the emergence or existence of a prostate tumor). Similarly, one can evaluate the integrity 103P3E8 nucleotide and amino acid sequences in a biological sample, in order to identify perturbations in the structure of these molecules such as insertions, deletions, substitutions and the like. The presence of one or more perturbations in 103P3E8 gene products in the sample is an indication of cancer susceptibility (or the emergence or existence of a tumor).

The invention also comprises methods for gauging tumor aggressiveness. In one embodiment, a method for gauging aggressiveness of a tumor comprises determining the level of 103P3E8 mRNA or 103P3E8 protein expressed by tumor cells, comparing the level so determined to the level of 103P3E8 mRNA or 103P3E8 protein expressed in a corresponding normal tissue taken from the same individual or a normal tissue reference sample, wherein the degree of 103P3E8 mRNA or 103P3E8 protein expression in the tumor sample relative to the normal sample indicates the degree of aggressiveness. In a specific embodiment, aggressiveness of a tumor is evaluated by determining the extent to which 103P3E8 is expressed in the tumor cells, with higher expression levels indicating more aggressive tumors. Another embodiment is the evaluation of the integrity of 103P3E8 nucleotide and amino acid sequences in a biological sample, in order to identify perturbations in the structure of these molecules such as insertions, deletions, substitutions and the like. The presence of one or more perturbations indicates more aggressive tumors.

Another embodiment of the invention is directed to methods for observing the progression of a malignancy in an individual over time. In one embodiment, methods for observing the progression of a malignancy in an individual over time comprise determining the level of 103P3E8 mRNA or 103P3E8 protein expressed by cells in a sample of the tumor, comparing the level so determined to the level of 103P3E8 mRNA or 103P3E8 protein expressed in an equivalent tissue sample taken from the same individual at a different time, wherein the degree of 103P3E8 mRNA or 103P3E8 protein expression in the tumor sample over time provides information on the progression of the cancer. In a specific embodiment, the progression of a cancer is evaluated by determining 103P3E8 expression in the tumor cells over time, where increased expression over time indicates a progression of the cancer. Also, one can evaluate the integrity 103P3E8 nucleotide and amino acid sequences in a biological sample in order to identify perturbations in the structure of these molecules such as insertions, deletions, substitutions and the like, where the presence of one or more perturbations indicates a progression of the cancer.

The above diagnostic approaches can be combined with any one of a wide variety of prognostic and diagnostic protocols known in the art. For example, another embodiment of the invention is directed to methods for observing a coincidence between the expression of 103P3E8 gene and 103P3E8 gene products (or perturbations in 103P3E8 gene and 103P3E8 gene products) and a factor that is associated with malignancy, as a means for diagnosing and prognosticating the status of a tissue sample. A wide variety of factors associated with malignancy can be utilized, such as the expression of genes associated with malignancy (e.g. PSA, PSCA and PSM expression for prostate cancer etc.) as well as gross cytological observations (see, e.g., Bocking et al., 1984, Anal. Quant. Cytol. 6(2):74-88; Epstein, 1995, Hum. Pathol. 26(2):223-9; Thorson et al., 1998, Mod. Pathol. 11(6):543-51; Baisden et al., 1999, Am. J. Surg. Pathol. 23(8):918-24). Methods for observing a coincidence between the expression of 103P3E8 gene and 103P3E8 gene products (or perturbations in 103P3E8 gene and 103P3E8 gene products) and another factor that is associated with malignancy are useful, for example, because the presence of a set of specific factors that coincide with disease provides information crucial for diagnosing and prognosticating the status of a tissue sample.

In one embodiment, methods for observing a coincidence between the expression of 103P3E8 gene and 103P3E8 gene products (or perturbations in 103P3E8 gene and 103P3E8 gene products) and another factor associated with malignancy entails detecting the overexpression of 103P3E8 mRNA or protein in a tissue sample, detecting the overexpression of PSA mRNA or protein in a tissue sample (or PSCA or PSM expression), and observing a coincidence of 103P3E8 mRNA or protein and PSA mRNA or protein overexpression (or PSCA or PSM expression). In a specific embodiment, the expression of 103P3E8 and PSA mRNA in prostate tissue is examined, where the coincidence of 103P3E8 and PSA mRNA overexpression in the sample indicates the existence of prostate cancer, prostate cancer susceptibility or the emergence or status of a prostate tumor.

Methods for detecting and quantifying the expression of 103P3E8 mRNA or protein are described herein, and standard nucleic acid and protein detection and quantification technologies are well known in the art. Standard methods for the detection and quantification of 103P3E8 mRNA include in situ hybridization using labeled 103P3E8 riboprobes, Northern blot and related techniques using 103P3E8 polynucleotide probes, RT-PCR analysis using primers specific for 103P3E8, and other amplification type detection methods, such as, for example, branched DNA, SISBA, TMA and the like. In a specific embodiment, semi-quantitative RT-PCR is used to detect and quantify 103P3E8 mRNA expression. Any number of primers capable of amplifying 103P3E8 can be used for this purpose, including but not limited to the various primer sets specifically described herein. In a specific embodiment, polyclonal or monoclonal antibodies specifically reactive with the wild-type 103P3E8 protein can be used in an immunohistochemical assay of biopsied tissue.

### IX.) Identification of Molecules That Interact With 103P3E8

The 103P3E8 protein and nucleic acid sequences disclosed herein allow a skilled artisan to identify proteins, small molecules and other agents that interact with 103P3E8, as well as pathways activated by 103P3E8 via any one of a variety of art accepted protocols. For example, one can utilize one of the so-called interaction trap systems (also referred to as the “two-hybrid assay”). In such systems, molecules interact and reconstitute a transcription factor which directs expression of a reporter gene, whereupon the expression of the reporter gene is assayed. Other systems identify protein-protein interactions in vivo through reconstitution of a eukaryotic transcriptional activator, see, e.g., U.S. Pat. Nos. 5,955,280 issued Sep. 21, 1999, 5,925,523 issued Jul. 20, 1999, 5,846,722 issued Dec. 8, 1998 and 6,004,746 issued Dec. 21, 1999. Algorithms are also available in the art for genome-based predictions of protein function (see, e.g., Marcotte, et al., Nature 402: Nov. 4, 1999, 83-86).

Alternatively one can screen peptide libraries to identify molecules that interact with 103P3E8 protein sequences. In such methods, peptides that bind to a molecule such as 103P3E8 are identified by screening libraries that encode a random or controlled collection of amino acids. Peptides encoded by the libraries are expressed as fusion proteins of bacteriophage coat proteins, the bacteriophage particles are then screened against the protein of interest.

Accordingly, peptides having a wide variety of uses, such as therapeutic, prognostic or diagnostic reagents, are thus identified without any prior information on the structure of the expected ligand or receptor molecule. Typical peptide libraries and screening methods that can be used to identify molecules that interact with 103P3E8 protein sequences are disclosed for example in U.S. Pat. Nos. 5,723,286 issued Mar. 3, 1998 and 5,733,731 issued Mar. 31, 1998.

Alternatively, cell lines that express 103P3E8 are used to identify protein-protein interactions mediated by 103P3E8. Such interactions can be examined using immunoprecipitation techniques (see, e.g., Hamilton BJ, et al. Biochem. Biophys. Res. Commun. 1999, 261:646-51). 103P3E8 protein can be immunoprecipitated from 103P3E8-expressing cell lines using anti-103P3E8 antibodies. Alternatively, antibodies against His-tag can be used in a cell line engineered to express 103P3E8 (vectors mentioned above). The immunoprecipitated complex can be examined for protein association by procedures such as Western blotting, ³⁵5-methionine labeling of proteins, protein microsequencing, silver staining and two-dimensional gel electrophoresis.

Small molecules and ligands that interact with 103P3E8 can be identified through related embodiments of such screening assays. For example, small molecules can be identified that interfere with protein function, including molecules that interfere with 103P3E8's ability to mediate phosphorylation and de-phosphorylation, interaction with DNA or RNA molecules as an indication of regulation of cell cycles, second messenger signaling or tumorigenesis. Similarly, small molecules that modulate ion channel, protein pump, or cell communication function of 103P3E8 are identified and used to treat patients that have a cancer that expresses the 103P3E8 antigen (see, e.g., Hille, B., Ionic Channels of Excitable Membranes 2^{nd} Ed., Sinauer Assoc., Sunderland, Mass., 1992). Moreover, ligands that regulate 103P3E8 function can be identified based on their ability to bind 103P3E8 and activate a reporter construct. Typical methods are discussed for example in U.S. Pat. No. 5,928,868 issued Jul. 27, 1999, and include methods for forming hybrid ligands in which at least one ligand is a small molecule. In an illustrative embodiment, cells engineered to express a fusion protein of 103P3E8 and a DNA-binding protein are used to co-express a fusion protein of a hybrid ligand/small molecule and a cDNA library transcriptional activator protein. The cells farther contain a reporter gene, the expression of which is conditioned on the proximity of the first and second fusion proteins to each other, an event that occurs only if the hybrid ligand binds to target sites on both hybrid proteins. Those cells that express the reporter gene are selected and the unknown small molecule or the unknown ligand is identified. This method provides a means of identifying both activators and inhibitors of 103P3E8.

An embodiment of this invention comprises a method of screening for a molecule that interacts with an 103P3E8 amino acid sequence shown in FIG. 2, FIG. 3 or FIG. 4, comprising the steps of contacting a population of molecules with the 103P3E8 amino acid sequence, allowing the population of molecules and the 103P3E8 amino acid sequence to interact under conditions that facilitate an interaction, determining the presence of a molecule that interacts with the 103P3E8 amino acid sequence, and then separating molecules that do not interact with the 103P3E8 amino acid sequence from molecules that do. In a specific embodiment, the method further comprises purifying a molecule that interacts with the 103P3E8 amino acid sequence. The identified molecule can be used to modulate a function performed by 103P3E8. In a preferred embodiment, the 103P3E8 amino acid sequence is contacted with a library of peptides.

### X.) Therapeutic Methods and Compositions

The identification of 103P3E8 as a protein that is normally expressed in a restricted set of tissues, but which is also expressed in prostate and other cancers, opens a number of therapeutic approaches to the treatment of such cancers. As discussed herein, it is possible that 103P3E8 functions as a transcription factor involved in activating tumor-promoting genes or repressing genes that block tumorigenesis.

Accordingly, therapeutic approaches that inhibit the activity of the 103P3E8 protein are useful for patients suffering from a cancer that expresses 103P3E8. These therapeutic approaches generally fall into two classes. One class comprises various methods for inhibiting the binding or association of the 103P3E8 protein with its binding partner or with other proteins. Another class comprises a variety of methods for inhibiting the transcription of the 103P3E8 gene or translation of 103P3E8 mRNA.

X.A.) 103P3E8 as a Target for Antibody-Based Therapy

103P3E8 is an attractive target for antibody-based therapeutic strategies. A number of antibody strategies are known in the art for targeting both extracellular and intracellular molecules (see, e.g., complement and ADCC mediated killing as well as the use of intrabodies). Because 103P3E8 is expressed by cancer cells of various lineages and not by corresponding normal cells, systemic administration of 103P3E8-immunoreactive compositions are prepared that exhibit excellent sensitivity without toxic, non-specific and/or non-target effects caused by binding of the immunoreactive composition to non-target organs and tissues. Antibodies specifically reactive with domains of 103P3E8 are useful to treat 103P3E8-expressing cancers systemically, either as conjugates with a toxin or therapeutic agent, or as naked antibodies capable of inhibiting cell proliferation or function.

103P3E8 antibodies can be introduced into a patient such that the antibody binds to 103P3E8 and modulates a function, such as an interaction with a binding partner, and consequently mediates destruction of the tumor cells and/or inhibits the growth of the tumor cells. Mechanisms by which such antibodies exert a therapeutic effect can include complement-mediated cytolysis, antibody-dependent cellular cytotoxicity, modulation of the physiological function of 103P3E8, inhibition of ligand binding or signal transduction pathways, modulation of tumor cell differentiation, alteration of tumor angiogenesis factor profiles, and/or apoptosis.

Those skilled in the art understand that antibodies can be used to specifically target and bind immunogenic molecules such as an immunogenic region of the 103P3E8 sequence shown in FIG. 2 or FIG. 4. In addition, skilled artisans understand that it is routine to conjugate antibodies to cytotoxic agents. When cytotoxic and/or therapeutic agents are delivered directly to cells, such as by conjugating them to antibodies specific for a molecule expressed by that cell (e.g. 103P3E8), the cytotoxic agent will exert its known biological effect (i.e. cytotoxicity) on those cells.

A wide variety of compositions and methods for using antibody-cytotoxic agent conjugates to kill cells are known in the art. In the context of cancers, typical methods entail administering to an animal having a tumor a biologically effective amount of a conjugate comprising a selected cytotoxic and/or therapeutic agent linked to a targeting agent (e.g. an anti-103P3E8 antibody) that binds to a marker (e.g. 103P3E8) expressed, accessible to binding or localized on the cell surfaces. A typical embodiment is a method of delivering a cytotoxic and/or therapeutic agent to a cell expressing 5 103P3E8, comprising conjugating the cytotoxic agent to an antibody that immunospecifically binds to a 103P3E8 epitope, and, exposing the cell to the antibody-agent conjugate. Another illustrative embodiment is a method of treating an individual suspected of suffering from metastasized cancer, comprising a step of administering parenterally to said individual a pharmaceutical composition comprising a therapeutically effective amount of an antibody conjugated to a cytotoxic and/or therapeutic agent.

Cancer immunotherapy using anti-103P3E8 antibodies can be done in accordance with various approaches that have been successfully employed in the treatment of other types of cancer, including but not limited to colon cancer (Arlen et al., 1998, Crit. Rev. Immunol. 18:133-138), multiple myeloma (Ozaki et al., 1997, Blood 90:3179-3186, Tsunenari et al., 1997, Blood 90:2437-2444), gastric cancer (Kasprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). Some therapeutic approaches involve conjugation of naked antibody to a toxin, such as the conjugation of Y⁹¹ or I¹³¹ to anti-CD20 antibodies (e.g., Zevalin™, IDEC Pharmaceuticals Corp. or Bexxar™, Coulter Pharmaceuticals), while others involve co-administration of antibodies and other therapeutic agents, such as Herceptin™ (trastuzumab) with paclitaxel (Genentech, Inc.). To treat prostate cancer, for example, 103P3E8 antibodies can be administered in conjunction with radiation, chemotherapy or hormone ablation.

Although 103P3E8 antibody therapy is useful for all stages of cancer, antibody therapy can be particularly appropriate in advanced or metastatic cancers. Treatment with the antibody therapy of the invention is indicated for patients who have received one or more rounds of chemotherapy. Alternatively, antibody therapy of the invention is combined with a chemotherapeutic or radiation regimen for patients who have not received chemotherapeutic treatment. Additionally, antibody therapy can enable the use of reduced dosages of concomitant chemotherapy, particularly for patients who do not tolerate the toxicity of the chemotherapeutic agent very well.

Cancer patients can be evaluated for the presence and level of 103P3E8 expression, preferably using immunohistochemical assessments of tumor tissue, quantitative 103P3E8 imaging, or other techniques that reliably indicate the presence and degree of 103P3E8 expression. Immunohistochemical analysis of tumor biopsies or surgical specimens is preferred for this purpose. Methods for immunohistochemical analysis of tumor tissues are well known in the art.

Anti-103P3E8 monoclonal antibodies that treat prostate and other cancers include those that initiate a potent immune response against the tumor or those that are directly cytotoxic. In this regard, anti-103P3E8 monoclonal antibodies (mAbs) can elicit tumor cell lysis by either complement-mediated or antibody-dependent cell cytotoxicity (ADCC) mechanisms, both of which require an intact Fc portion of the immunoglobulin molecule for interaction with effector cell Fc receptor sites on complement proteins. In addition, anti-103P3E8 mAbs that exert a direct biological effect on tumor growth are useful to treat cancers that express 103P3E8. Mechanisms by which directly cytotoxic mAbs act include: inhibition of cell growth, modulation of cellular differentiation, modulation of tumor angiogenesis factor profiles, and the induction of apoptosis. The mechanism(s) by which a particular anti-103P3E8 mAb exerts an anti-tumor effect is evaluated using any number of in vitro assays that evaluate cell death such as ADCC, ADMMC, complement-mediated cell lysis, and so forth, as is generally known in the art.

In some patients, the use of murine or other non-human monoclonal antibodies, or human/mouse chimeric mAbs can induce moderate to strong immune responses against the non-human antibody. This can result in clearance of the antibody from circulation and reduced efficacy. In the most severe cases, such an immune response can lead to the extensive formation of immune complexes which, potentially, can cause renal failure. Accordingly, preferred monoclonal antibodies used in the therapeutic methods of the invention are those that are either fully human or humanized and that bind specifically to the target 103P3E8 antigen with high affinity but exhibit low or no antigenicity in the patient.

Therapeutic methods of the invention contemplate the administration of single anti-103P3E8 mAbs as well as combinations, or cocktails, of different mAbs. Such mAb cocktails can have certain advantages inasmuch as they contain mAbs that target different epitopes, exploit different effector mechanisms or combine directly cytotoxic mAbs with mAbs that rely on immune effector functionality. Such mAbs in combination can exhibit synergistic therapeutic effects. In addition, anti-103P3E8 mAbs can be administered concomitantly with other therapeutic modalities, including but not limited to various chemotherapeutic agents, androgen-blockers, immune modulators (e.g., IL-2, GM-CSF), surgery or radiation. The anti-103P3E8 mAbs are administered in their “naked”or unconjugated form, or can have a therapeutic agent(s) conjugated to them.

Anti-103P3E8 antibody formulations are administered via any route capable of delivering the antibodies to a tumor cell. Routes of administration include, but are not limited to, intravenous, intraperitoneal, intramuscular, intratumor, intradermal, and the like. Treatment generally involves repeated administration of the anti-103P3E8 antibody preparation, via an acceptable route of administration such as intravenous injection (IV), typically at a dose in the range of about 0.1 to about 10 mg/kg body weight. In general, doses in the range of 10-500 mg mAb per week are effective and well tolerated.

Based on clinical experience with the Herceptin mAb in the treatment of metastatic breast cancer, an initial loading dose of approximately 4 mg/kg patient body weight IV, followed by weekly doses of about 2 mg/kg IV of the anti- 103P3E8 mAb preparation represents an acceptable dosing regimen. Preferably, the initial loading dose is administered as a 90 minute or longer infusion. The periodic maintenance dose is administered as a 30 minute or longer infusion, provided the initial dose was well tolerated. As appreciated by those of skill in the art, various factors can influence the ideal dose regimen in a particular case. Such factors include, for example, the binding affinity and half life of the Ab or mAbs used, the degree of 103P3E8 expression in the patient, the extent of circulating shed 103P3E8 antigen, the desired steady-state antibody concentration level, frequency of treatment, and the influence of chemotherapeutic or other agents used in combination with the treatment method of the invention, as well as the health status of a particular patient.

Optionally, patients should be evaluated for the levels of 103P3E8 in a given sample (e.g. the levels of circulating 103P3E8 antigen and/or 103P3E8 expressing cells) in order to assist in the determination of the most effective dosing regimen, etc. Such evaluations are also used for monitoring purposes throughout therapy, and are useful to gauge therapeutic success in combination with the evaluation of other parameters (such as serum PSA levels in prostate cancer therapy).

X.B.) Anti-Cancer Vaccines

The invention further provides cancer vaccines comprising a 103P3E8-related protein or 103P3E8-related nucleic acid. In view of the expression of 103P3E8, cancer vaccines prevent and/or treat 103P3E8-expressing cancers without creating non-specific effects on non-target tissues. The use of a tumor antigen in a vaccine that generates humoral and/or cell-mediated immune responses as anti-cancer therapy is well known in the art and has been employed in prostate cancer using human PSMA and rodent PAP immunogens (Hodge et al., 1995, Int. J. Cancer 63:231-237; Fong et al., 1997, J. Immunol. 159:3113-3117).

Genetic immunization methods can be employed to generate prophylactic or therapeutic humoral and cellular immune responses directed against cancer cells expressing 103P3E8. Constructs comprising DNA encoding a 103P3E8-related protein/immunogen and appropriate regulatory sequences can be injected directly into muscle or skin of an individual, such that the cells of the muscle or skin take-up the construct and express the encoded 103P3E8 protein/immunogen. Alternatively, a vaccine comprises a 103P3E8-related protein. Expression of the 103P3E8-related protein immunogen results in the generation of prophylactic or therapeutic humoral and cellular immunity against cells that bear 103P3E8 protein. Various prophylactic and therapeutic genetic immunization techniques known in the art can be used (for review, see information and references published at Internet address www.genweb.com).

Such methods can be readily practiced by employing a 103P3E8-related protein, or an 103P3E8-encoding nucleic acid molecule and recombinant vectors capable of expressing and presenting the 103P3E8 immunogen (which typically comprises a number of antibody or T cell epitopes). Skilled artisans understand that a wide variety of vaccine systems for delivery of immunoreactive epitopes are known in the art (see, e.g., Heryln et al., Ann Med 1999 Feb 31(l):66-78; Maruyama et al., Cancer Immunol Immunother 2000 Jun 49(3):123-32) Briefly, such methods of generating an immune response (e.g. humoral and/or cell-mediated) in a mammal, comprise the steps of: exposing the mammal's immune system to an immunoreactive epitope (e.g. an epitope present in the 103P3E8 protein shown in SEQ ID NO: 2 or analog or homolog thereof) so that the mammal generates an immune response that is specific for that epitope (e.g. generates antibodies that specifically recognize that epitope). In a preferred method, the 103P3E8 immunogen contains a biological motif.

CTL epitopes can be determined using specific algorithms to identify peptides within 103P3E8 protein that are capable of optimally binding to specified HLA alleles (e.g., Table IV (A) and Table IV (B); Epimer™ and Epimatrix™, Brown University (http://www.brown.edu/Research/TB-HIV_Lab/epimatrix/epimatrix.html); and, BIMAS, (http://bimas.dcrt.nih.gov/). In a preferred embodiment, the 103P3E8 immunogen contains one or more amino acid sequences identified using one of the pertinent analytical techniques well known in the art, such as the sequences shown in Tables V-XVIII or a peptide of 8, 9, 10 or 11 amino acids specified by an HLA Class I motif (e.g., Table IV (A)) and/or a peptide of at least 9 amino acids that comprises an HLA Class II motif (e.g., Table IV (B)). As is appreciated in the art, the HLA Class I binding groove is essentially closed ended so that peptides of only a particular size range can fit into the groove and be bound, generally HLA Class I epitopes are 8, 9, 10, or 11 amino acids long. In contrast, the HLA Class II binding groove is essentially open ended; therefore a peptide of about 9 or more amino acids can be bound by an HLA Class II molecule. Due to the binding groove differences between HLA Class I and II, HLA Class I motifs are length specific, i.e., position two of a Class I motif is the second amino acid in an amino to carboxyl direction of the peptide. The amino acid positions in a Class II motif are relative only to each other, not the overall peptide, i.e., additional amino acids can be attached to the amino and/or carboxyl termini of a motif-bearing sequence. HLA Class II epitopes are often 9, 10, 11, 12, 13, 14, 15, 16, 17, 18, 19, 20, 21, 22, 23, 24, or 25 amino acids long, or longer than 25 amino acids.

A wide variety of methods for generating an immune response in a mammal are known in the art (for example as the first step in the generation of hybridomas). Methods of generating an immune response in a mammal comprise exposing the mammal's immune system to an immunogenic epitope on a protein (e.g. the 103P3E8 protein) so that an immune response is generated. A typical embodiment consists of a method for generating an immune response to 103P3E8 in a host, by contacting the host with a sufficient amount of at least one 103P3E8 B cell or cytotoxic T-cell epitope or analog thereof; and at least one periodic interval thereafter re-contacting the host with the 103P3E8 B cell or cytotoxic T-cell epitope or analog thereof. A specific embodiment consists of a method of generating an immune response against a 103P3E8-related protein or a man-made multiepitopic peptide comprising: administering 103P3E8 immunogen (e.g. the 103P3E8 protein or a peptide fragment thereof, an 103P3E8 fusion protein or analog etc.) in a vaccine preparation to a human or another mammal. Typically, such vaccine preparations further contain a suitable adjuvant (see, e.g., U.S. Pat. No. 6,146,635) or a universal helper epitope such as a PADRE™ peptide (Epimmune Inc., San Diego, Calif.; see, e.g., Alexander et al., J. Immunol. 2000 164(3); 164(3): 1625-1633; Alexander et al., Immunity 1994 1(9): 751-761 and Alexander et al., Immunol. Res. 1998 18(2): 79-92). An alternative method comprises generating an immune response in an individual against a 103P3E8 immunogen by: administering in vivo to muscle or skin of the individual's body a DNA molecule that comprises a DNA sequence that encodes an 103P3E8 immunogen, the DNA sequence operatively linked to regulatory sequences which control the expression of the DNA sequence; wherein the DNA molecule is taken up by cells, the DNA sequence is expressed in the cells and an immune response is generated against the immunogen (see, e.g., U.S. Pat. No. 5,962,428). The DNA can be dissociated from an infectious agent. Optionally a genetic vaccine facilitator such as anionic lipids; saponins; lectins; estrogenic compounds; hydroxylated lower alkyls; dimethyl sulfoxide; and urea is also administered.

Thus, viral gene delivery systems are used to deliver a 103P3E8-related nucleic acid molecule. Various viral gene delivery systems that can be used in the practice of the invention include, but are not limited to, vaccinia, fowlpox, canarypox, adenovirus, influenza, poliovirus, adeno-associated virus, lentivirus, and sindbis virus (Restifo, 1996, Curr. Opin. Immunol. 8:658-663). Non-viral delivery systems can also be employed by introducing naked DNA encoding a 103P3E8-related protein into the patient (e.g., intramuscularly or intradermally) to induce an anti-tumor response. In one embodiment, the full-length human 103P3E8 cDNA is employed. In another embodiment, 103P3E8 nucleic acid molecules encoding specific cytotoxic T lymphocyte (CTL) and/or antibody epitopes are employed.

Various ex vivo strategies can also be employed to generate an immune response. One approach involves the use of antigen presenting cells (APCs) such as dendritic cells to present 103P3E8 antigen to a patient's immune system. Dendritic cells express MHC class I and II molecules, B7 co-stimulator, and IL-12, and are thus highly specialized antigen presenting cells. In prostate cancer, autologous dendritic cells pulsed with peptides of the prostate-specific membrane antigen (PSMA) are being used in a Phase I clinical trial to stimulate prostate cancer patients' immune systems (Tjoa et al., 1996, Prostate 28:65-69; Murphy et al., 1996, Prostate 29:371-380). Thus, dendritic cells can be used to present 103P3E8 peptides to T cells in the context of MHC class I or II molecules. In one embodiment, autologous dendritic cells are pulsed with 103P3E8 peptides capable of binding to MHC class I and/or class II molecules. In another embodiment, dendritic cells are pulsed with the complete 103P3E8 protein. Yet another embodiment involves engineering the overexpression of the 103P3E8 gene in dendritic cells using various implementing vectors known in the art, such as adenovirus (Arthur et al., 1997, Cancer Gene Ther. 4:17-25), retrovirus (Henderson et al., 1996, Cancer Res. 56:3763-3770), lentivirus, adeno-associated virus, DNA transfection (Ribas et al., 1997, Cancer Res. 57:2865-2869), or tumor-derived RNA transfection (Ashley et al., 1997, J. Exp. Med. 186:1177-1182). Cells that express 103P3E8 can also be engineered to express immune modulators, such as GM-CSF, and used as immunizing agents.

Anti-idiotypic anti-103P3E8 antibodies can also be used in anti-cancer therapy as a vaccine for inducing an immune response to cells expressing a 103P3E8-related protein. In particular, the generation of anti-idiotypic antibodies is well known in the art; this methodology can readily be adapted to generate anti-idiotypic anti-103P3E8 antibodies that mimic an epitope on a 103P3E8-related protein (see, for example, Wagner et al., 1997, Hybridoma 16: 33-40; Foon et al., 1995, J. Clin. Invest. 96:334-342; Herlyn et al., 1996, Cancer Immunol. Immunother. 43:65-76). Such an anti-idiotypic antibody can be used in cancer vaccine strategies.

### XI.) Inhibition of 103P3E8 Protein Function

The invention includes various methods and compositions for inhibiting the binding of 103P3E8 to its binding partner or its association with other protein(s) as well as methods for inhibiting 103P3E8 function.

XI.A.) Inhibition of 103P3E8 With Intracellular Antibodies

In one approach, a recombinant vector that encodes single chain antibodies that specifically bind to 103P3E8 are introduced into 103P3E8 expressing cells via gene transfer technologies. Accordingly, the encoded single chain anti-103P3E8 antibody is expressed intracellularly, binds to 103P3E8 protein, and thereby inhibits its function. Methods for engineering such intracellular single chain antibodies are well known. Such intracellular antibodies, also known as “intrabodies”, are specifically targeted to a particular compartment within the cell, providing control over where the inhibitory activity of the treatment is focused. This technology has been successfully applied in the art (for review, see Richardson and Marasco, 1995, TIBTECH vol. 13). Intrabodies have been shown to virtually eliminate the expression of otherwise abundant cell surface receptors (see, e.g., Richardson et al., 1995, Proc. Natl. Acad. Sci. USA 92: 3137-3141; Beerli et al., 1994, J. Biol. Chem. 289: 23931-23936; Deshane et al., 1994, Gene Ther. 1: 332-337).

Single chain antibodies comprise the variable domains of the heavy and light chain joined by a flexible linker polypeptide, and are expressed as a single polypeptide. Optionally, single chain antibodies are expressed as a single chain variable region fragment joined to the light chain constant region. Well-known intracellular trafficking signals are engineered into recombinant polynucleotide vectors encoding such single chain antibodies in order to precisely target the intrabody to the desired intracellular compartment. For example, intrabodies targeted to the endoplasmic reticulum (ER) are engineered to incorporate a leader peptide and, optionally, a C-terminal ER retention signal, such as the KDEL amino acid motif. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

In one embodiment, intrabodies are used to capture 103P3E8 in the nucleus, thereby preventing its activity within the nucleus. Nuclear targeting signals are engineered into such 103P3E8 intrabodies in order to achieve the desired targeting. Such 103P3E8 intrabodies are designed to bind specifically to a particular 103P3E8 domain. In another embodiment, cytosolic intrabodies that specifically bind to the 103P3E8 protein are used to prevent 103P3E8 from gaining access to the nucleus, thereby preventing it from exerting any biological activity within the nucleus (e.g., preventing 103P3E8 from forming transcription complexes with other factors).

In order to specifically direct the expression of such intrabodies to particular cells, the transcription of the intrabody is placed under the regulatory control of an appropriate tumor-specific promoter and/or enhancer. In order to target intrabody expression specifically to prostate, for example, the PSA promoter and/or promoter/enhancer can be utilized (See, for example, U.S. Pat. No. 5,919,652 issued Jul. 6, 1999).

XI.B.) Inhibition of 103P3E8 with Recombinant Proteins

In another approach, recombinant molecules bind to 103P3E8 and thereby inhibit 103P3E8 function. For example, these recombinant molecules prevent or inhibit 103P3E8 from accessing/binding to its binding partner(s) or associating with other protein(s). Such recombinant molecules can, for example, contain the reactive part(s) of a 103P3E8 specific antibody molecule. In a particular embodiment, the 103P3E8 binding domain of a 103P3E8 binding partner is engineered into a dimeric fusion protein, whereby the fusion protein comprises two 103P3E8 ligand binding domains linked to the Fc portion of a human IgG, such as human IgG1. Such IgG portion can contain, for example, the C_{H}2 and C_{H}3 domains and the hinge region, but not the C_{H}1 domain. Such dimeric fusion proteins are administered in soluble form to patients suffering from a cancer associated with the expression of 103P3E8, whereby the dimeric fusion protein specifically binds to 103P3E8 and blocks 103P3E8 interaction with a binding partner. Such dimeric fusion proteins are further combined into multimeric proteins using known antibody linking technologies.

XI.C.) Inhibition of 103P3E8 Transcription or Translation

The present invention also comprises various methods and compositions for inhibiting the transcription of the 103P3E8 gene. Similarly, the invention also provides methods and compositions for inhibiting the translation of 103P3E8 mRNA into protein.

In one approach, a method of inhibiting the transcription of the 103P3E8 gene comprises contacting the 103P3E8 gene with a 103P3E8 antisense polynucleotide. In another approach, a method of inhibiting 103P3E8 mRNA translation comprises contacting the 103P3E8 mRNA with an antisense polynucleotide. In another approach, a 103P3E8 specific ribozyme is used to cleave the 103P3E8 message, thereby inhibiting translation. Such antisense and ribozyme based methods can also be directed to the regulatory regions of the 103P3E8 gene, such as the 103P3E8 promoter and/or enhancer elements. Similarly, proteins capable of inhibiting a 103P3E8 gene transcription factor are used to inhibit 103P3E8 mRNA transcription. The various polynucleotides and compositions useful in the aforementioned methods have been described above. The use of antisense and ribozyme molecules to inhibit transcription and translation is well known in the art.

Other factors that inhibit the transcription of 103P3E8 by interfering with 103P3E8 transcriptional activation are also useful to treat cancers expressing 103P3E8. Similarly, factors that interfere with 103P3E8 processing are useful to treat cancers that express 103P3E8. Cancer treatment methods utilizing such factors are also within the scope of the invention.

XI.D.) General Considerations for Therapeutic Strategies

Gene transfer and gene therapy technologies can be used to deliver therapeutic polynucleotide molecules to tumor cells synthesizing 103P3E8 (i.e., antisense, ribozyme, polynucleotides encoding intrabodies and other 103P3E8 inhibitory molecules). A number of gene therapy approaches are known in the art. Recombinant vectors encoding 103P3E8 antisense polynucleotides, ribozymes, factors capable of interfering with 103P3E8 transcription, and so forth, can be delivered to target tumor cells using such gene therapy approaches.

The above therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

The anti-tumor activity of a particular composition (e.g., antisense, ribozyme, intrabody), or a combination of such compositions, can be evaluated using various in vitro and in vivo assay systems. In vitro assays that evaluate therapeutic activity include cell growth assays, soft agar assays and other assays indicative of tumor promoting activity, binding assays capable of determining the extent to which a therapeutic composition will inhibit the binding of 103P3E8 to a binding partner, etc.

In vivo, the effect of a 103P3E8 therapeutic composition can be evaluated in a suitable animal model. For example, xenogenic prostate cancer models can be used, wherein human prostate cancer explants or passaged xenograft tissues are introduced into immune compromised animals, such as nude or SCID mice (Klein et al., 1997, Nature Medicine 3: 402-408). For example, PCT patent application Ser. No. WO98/16628, Sawyers et al., published Apr. 23, 1998, describes various xenograft models of human prostate cancer capable of recapitulating the development of primary tumors, micrometastasis, and the formation of osteoblastic metastases characteristic of late stage disease. Efficacy can be predicted using assays that measure inhibition of tumor formation, tumor regression or metastasis, and the like.

In vivo assays that evaluate the promotion of apoptosis are useful in evaluating therapeutic compositions. In one embodiment, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

Therapeutic formulations can be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A preferred formulation for intravenous injection comprises the therapeutic composition in a solution of preserved bacteriostatic water, sterile unpreserved water, and/or diluted in polyvinylchloride or polyethylene bags containing 0.9% sterile Sodium Chloride for Injection, USP. Therapeutic protein preparations can be lyophilized and stored as sterile powders, preferably under vacuum, and then reconstituted in bacteriostatic water (containing for example, benzyl alcohol preservative) or in sterile water prior to injection.

Dosages and administration protocols for the treatment of cancers using the foregoing methods will vary with the method and the target cancer, and will generally depend on a number of other factors appreciated in the art.

### XII.) Kits

For use in the diagnostic and therapeutic applications described herein, kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise a probe that is or can be detectably labeled. Such probe can be an antibody or polynucleotide specific for a 103P3E8-related protein or a 103P3E8 gene or message, respectively. Where the method utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label. The kit can include all or part of the amino acid sequence of FIG. 2 FIG. 3 or FIG. 4 or analogs thereof, or a nucleic acid molecule that encodes such amino acid sequences.

The kit of the invention will typically comprise the container described above and one or more other containers comprising materials desirable from a commercial and user standpoint, including buffers, diluents, filters, needles, syringes, and package inserts with instructions for use.

A label can be present on the container to indicate that the composition is used for a specific therapy or non-therapeutic application, and can also indicate directions for either in vivo or in vitro use, such as those described above. Directions and or other information can also be included on an insert which is included with the kit.

### EXAMPLES

Various aspects of the invention are further described and illustrated by way of the several examples that follow, none of which are intended to limit the scope of the invention.

### Example 1: SSH-Generated Isolation of a cDNA Fragment of the 103P3E8 Gene

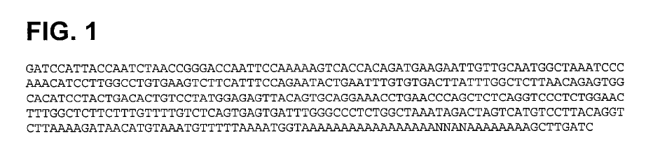

The SSH cDNA fragment 103P3E8 (FIG. 1) was derived from a subtraction utilizing the xenografts LAPC4AD (21 days after castration) minus LAPC4AD (non-castrated mouse). The cDNA clone 103P3E8-clone 7 (FIG. 4) was isolated from an LAPC4AD cDNA library (Lambda ZAP Express, Stratagene).

The 103P3E8 clone 7 cDNA encodes a 625 amino acid ORF (5′ open) predicted to be localized to the nucleus (PSORT) and shows homology the p21ras-like G proteins at the carboxyl-terminus and some homology to intermediate filament proteins. The 103P3E8 clone 7 protein is encompassed within a protein sequence (FIG. 3, underlined region). The clone 7 protein is truncated at the C-terminus and could represent the cancer form of the protein since it was cloned from an LAPC4AD library as opposed to a normal prostate library. It also has a nuclear localization signal and is predicted to be localized to the nucleus using the PSORT program (http://nibb.ac.jp:8800/form.html).

### Materials and Methods

LAPC Xenografts and Human Tissues:

LAPC xenografts were obtained from Dr. Charles Sawyers (UCLA) and generated as described (Klein et al, 1997, Nature Med. 3: 402-408; Craft et al., 1999, Cancer Res. 59: 5030-5036). Androgen dependent and independent LAPC-4 xenografts LAPC-4 AD and AI, respectively) and LAPC-9 AD and AI xenografts were grown in male SCID mice and were passaged as small tissue chunks in recipient males. LAPC-4 and -9 AI xenografts were derived from LAPC-4 or -9 AD tumors, respectively. To generate the AI xenografts, male mice bearing AD tumors were castrated and maintained for 2-3 months. After the tumors re-grew, the tumors were harvested and passaged in castrated males or in female SCID mice.

Cell Lines:

Human cell lines (e.g., HeLa) were obtained from the ATCC and were maintained in DMEM with 5% fetal calf serum.

RNA Isolation:

Tumor tissue and cell lines were homogenized in Trizol reagent (Life Technologies, Gibco BRL) using 10 ml/g tissue or 10 ml/10⁸ cells to isolate total RNA. Poly A RNA was purified from total RNA using Qiagen's Oligotex mRNA Mini and Midi kits. Total and mRNA were quantified by spectrophotometric analysis (O.D. 260/280 nm) and analyzed by gel electrophoresis.

Oligonucleotides:

The following HPLC purified oligonucleotides were used.

DPNCDN (cDNA synthesis primer):

5′ TTTTGATCAAGCTT₃₀3′ (SEQ ID NO: 7)

Adaptor 1:

5′ CTAATACGACTCACTATAGGGCTCGAGCGGCCGCCCGGGCAG3′ (SEQ ID NO: 8) 3′ GGCCCGTCCTAG5′ (SEQ ID NO: 9)

Adaptor 2:

5′ GTAATACGACTCACTATAGGGCAGCGTGGTCGCGGCCGAG3′ (SEQ ID NO:10) 3′ CGGCTCCTAG5′ (SEQ ID NO: 11)

PCR primer 1:

5′ CTAATACGACTCACTATAGGGC3′ (SEQ ID NO: 12)

Nested primer (NP)1:

5′ TCGAGCGGCCGCCCGGGCAGGA3′ (SEQ ID NO: 13)

Nested primer (NP)2:

5′ AGCGTGGTCGCGGCCGAGGA3′ (SEQ ID NO: 14)

Suppression Subtractive Hybridization:

Suppression Subtractive Hybridization (SSH) was used to identify cDNAs corresponding to genes that may be differentially expressed in prostate cancer. The SSH reaction utilized cDNA from two LAPC-4 AD xenografts. Specifically, to isolate genes that are involved in the progression of androgen dependent (AD) prostate cancer to androgen independent (AI) cancer, an experiment was conducted with the LAPC-4 AD xenograft in male SCID mice. Mice that harbored LAPC-4 AD xenografts were castrated when the tumors reached a size of 1 cm in diameter. The tumors regressed in size and temporarily stopped producing the androgen dependent protein PSA. Seven to fourteen days post-castration, PSA levels were detectable again in the blood of the mice. Eventually the tumors develop an AI phenotype and start growing again in the castrated males. Tumors were harvested at different time points after castration to identify genes that are turned on or off during the transition to androgen independence.

The gene 103P3E8 was derived from an LAPC-4 AD tumor (21 days post-castration) minus an LAPC-4 AD tumor (grown in intact male mouse) subtraction. The SSH DNA sequence (FIG. 1) was identified.

The cDNA derived from an LAPC-4 AD tumor (21 days post-castration) was used as the source of the “tester” cDNA, while the cDNA from the LAPC-4 AD tumor (grown in intact male mouse) was used as the source of the “driver” cDNA. Double stranded cDNAs corresponding to tester and driver cDNAs were synthesized from 2 μg of poly(A)⁺RNA isolated from the relevant xenograft tissue, as described above, using CLONTECH's PCR-Select cDNA Subtraction Kit and 1 ng of oligonucleotide DPNCDN as primer. First- and second-strand synthesis were carried out as described in the Kit's user manual protocol (CLONTECH Protocol No. PT1117-1, Catalog No. K1804-1). The resulting cDNA was digested with Dpn II for 3 hrs at 37° C. Digested cDNA was extracted with phenol/chloroform (1: 1) and ethanol precipitated.

Driver cDNA was generated by combining in a 1:1 ratio Dpn II digested cDNA from the relevant xenograft source (see above) with a mix of digested cDNAs derived from the human cell lines HeLa, 293, A431, Colo205, and mouse liver.

Tester cDNA was generated by diluting 1 μl of Dpn II digested cDNA from the relevant xenograft source (see above) (400 ng) in 5 μl of water. The diluted cDNA (2 μl 160 ng) was then ligated to 2 μl of Adaptor 1 and Adaptor 2 (10 μM), in separate ligation reactions, in a total volume of 10 μl at 16° C. overnight, using 400u of T4 DNA ligase (CLONTECH). Ligation was terminated with 1 μl of 0.2 M EDTA and heating at 72° C. for 5 min.

The first hybridization was performed by adding 1.5 μl (600 ng) of driver cDNA to each of two tubes containing 1.5 μl (20 ng) Adaptor 1- and Adaptor 2-ligated tester cDNA. In a final volume of 4 μl, the samples were overlaid with mineral oil, denatured in an MJ Research thermal cycler at 98° C. for 1.5 minutes, and then were allowed to hybridize for 8 hrs at 68° C. The two hybridizations were then mixed together with an additional 1 μl of fresh denatured driver cDNA and were allowed to hybridize overnight at 68° C. The second hybridization was then diluted in 200 μl of 20 mM Hepes, pH 8.3, 50 mM NaCl, 0.2 mM EDTA, heated at 70° C. for 7 min. and stored at −20° C.

PCR Amplification, Cloning and Sequencing of Gene Fragments Generated from SSH:

To amplify gene fragments resulting from SSH reactions, two PCR amplifications were performed. In the primary PCR reaction 1 μl of the diluted final hybridization mix was added to 1 μl of PCR primer 1 (10 μM), 0.5 μl dNTP mix (10 μM), 2.5 μl 10× reaction buffer (CLONTECH) and 10 0.5 μl 50× Advantage cDNA polymerase Mix (CLONTECH) in a final volume of 25 μl. PCR 1 was conducted using the following conditions: 75° C. for 5 min., 94° C. for 25 sec., then 27 cycles of 94° C. for 10 sec, 66° C. for 30 sec, 72° C. for 1.5 min. Five separate primary PCR reactions were performed for each experiment. The products were pooled and diluted 1:10 with water. For the secondary PCR reaction, 1 μl from the pooled and diluted primary PCR reaction was added to the same reaction mix as used for PCR 1, except that primers NP1 and NP2 (10 μM) were used instead of PCR primer 1. PCR 2 was performed using 10-12 cycles of 94° C. for 10 sec, 68° C. for 30 sec, and 72° C. for 1.5 minutes. The PCR products were analyzed using 2% agarose gel electrophoresis.

The PCR products were inserted into pCR2.1 using the T/A vector cloning kit (Invitrogen).

Transformed E. coli were subjected to blue/white and ampicillin selection. White colonies were picked and arrayed into 96 well plates and were grown in liquid culture overnight. To identify inserts, PCR amplification was performed on 1 ml of bacterial culture using the conditions of PCR1 and NP1 and NP2 as primers. PCR products were analyzed using 2% agarose gel electrophoresis.

Bacterial clones were stored in 20% glycerol in a 96 well format. Plasmid DNA was prepared, sequenced, and subjected to nucleic acid homology searches of the GenBank, dBest, and NCI-CGAP databases.

RT-PCR Expression Analysis:

First strand cDNAs can be generated from 1 μg of mRNA with oligo (dT) 12-18 priming using the Gibco-BRL Superscript Preamplification system. The manufacturer's protocol was used which included an incubation for 50 min at 42° C. with reverse transcriptase followed by RNAse H treatment at 37° C. for 20 min. After completing the reaction, the volume can be increased to 200 μl with water prior to normalization. First strand cDNAs from 16 different normal human tissues can be obtained from Clontech.

Normalization of the first strand cDNAs from multiple tissues was performed by using the primers 5′ atatcgccgcgctcgtcgtcgacaa3′ (SEQ ID NO: 15) and 5'agccacacgcagctcattgtagaagg 3′ (SEQ ID NO: 16) to amplify β-actin. First strand cDNA (5 μl) were amplified in a total volume of 50 μl containing 0.4 μM primers, 0.2 μM each dNTPs, 1XPCR buffer (Clontech, 10 mM Tris-HCL, 1.5 mM MgCl₂, 50 mM KCl, pH8.3) and 1X Klentaq DNA polymerase (Clontech). Five μl of the PCR reaction can be removed at 18, 20, and 22 cycles and used for agarose gel electrophoresis. PCR was performed using an MJ Research thermal cycler under the following conditions: Initial denaturation can be at 94° C. for 15 sec, followed by a 18, 20, and 22 cycles of 94° C. for 15, 65° C. for 2 min, 72° C. for 5 sec. A final extension at 72° C. was carried out for 2 min. After agarose gel electrophoresis, the band intensities of the 283 b.p. β-actin bands from multiple tissues were compared by visual inspection. Dilution factors for the first strand cDNAs were calculated to result in equal m-actin band intensities in all tissues after 22 cycles of PCR. Three rounds of normalization can be required to achieve equal band intensities in all tissues after 22 cycles of PCR.

To determine expression levels of the 103P3E8 gene, 5 μl of normalized first strand cDNA were analyzed by PCR using 26, and 30 cycles of amplification. Semi-quantitative expression analysis can be achieved by comparing the PCR products at cycle numbers that give light band intensities.

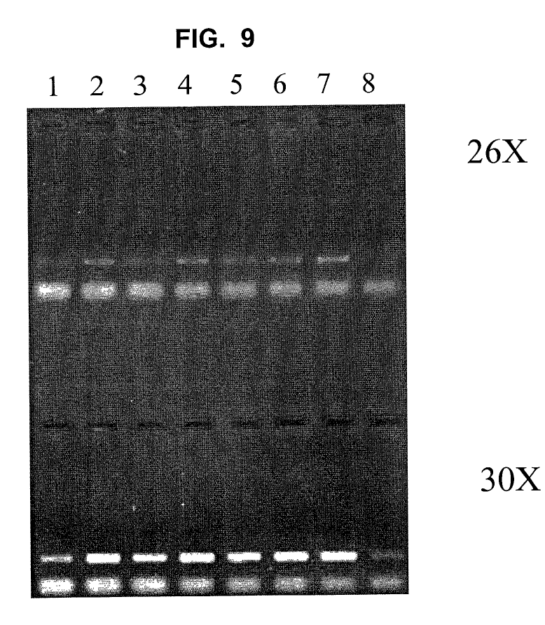

A typical RT-PCR expression analysis is shown in FIG. 9. RT-PCR expression analysis was performed on first strand cDNAs generated using pools of tissues from multiple samples. The cDNAs were shown to be normalized using beta-actin PCR. Expression of 103P3E8 was observed in prostate cancer xenografts, prostate cancer tissue pools, colon cancer tissue pools, kidney cancer tissue pools, and bladder cancer tissue pools.

### Example 2: Full Length Clonin2 of 103P3E8

To isolate genes that are involved in the progression of androgen dependent (AD) prostate cancer to androgen independent (AI) cancer, an experiment was conducted with the LAPC-4 AD xenograft in male SCID mice. Mice that harbored LAPC-4 AD xenografts were castrated when the tumors reached a size of 1 cm in diameter. The tumors regressed in size and temporarily stopped producing the androgen dependent protein PSA. Seven to fourteen days post-castration, PSA levels were detectable again in the blood of the mice. Eventually the tumors develop an AI phenotype and start growing again in the castrated males. Tumors were harvested at different time points after castration to identify genes that are turned on or off during the transition to androgen independence.

The gene 103P3E8 was derived from an LAPC-4AD (21 days post-castration) minus LAPC-4 AD (no castration) subtraction. The SSH DNA sequence (FIG. 1) was designated 103P3E8. cDNA clone 103P3E8-clone 7 (FIG. 4) was identified by screening an LAPC4AD cDNA library (Lambda ZAP Express, Stratagene) using the 103P3E8 SSH DNA as a probe.

103P3E8 clone 7 cDNA was deposited under the terms of the Budapest Treaty on Feb. 2, 2000, with the American Type Culture Collection (ATCC; 10801 University Blvd., Manassas, Va. 20110-2209 USA) as plasmid p103P3E8-7, and has been assigned Designation No. PTA-1 262.

### Example 3: Chromosomal Mapping of the 103P3E8 Gene

The chromosomal localization of 103P3E8 was determined using the NCBI Human Genome web site (http://www.ncbi.nlm.nih.gov/genome/seq/page.cgi?F=HsBlast.html&&ORG=Hs) and confirmed using the Coriell (Camden, N.J.) Human-Rodent Somatic Cell Hybrid Panel #2-version 3. The mapping program placed 103P3E8 on chromosome 9q13-q21.1, a genomic region found to be rearranged in certain cancers. Chromosomal localization of 103P3E8 was also determined by UniGene analysis. Bioinformatics analysis of the UniGene (Hs.34145) corresponding to 103P3E8 (identified using EST AA601537) also indicates that this gene maps to chromosome 9q13-q21 (UniGene database in NCBI at http://www.ncbi.nlm.nih.gov/UniGene/Hs.Home.html). The location of the 103P3E8 gene on chromosome 9 was confirmed by PCR analysis of the Coriell Mapping Panel #2 (Coriell Medical Research Institute).

### Example 4: Expression analysis of 103P3E8 in normal tissues, cancer cell lines and patient samples

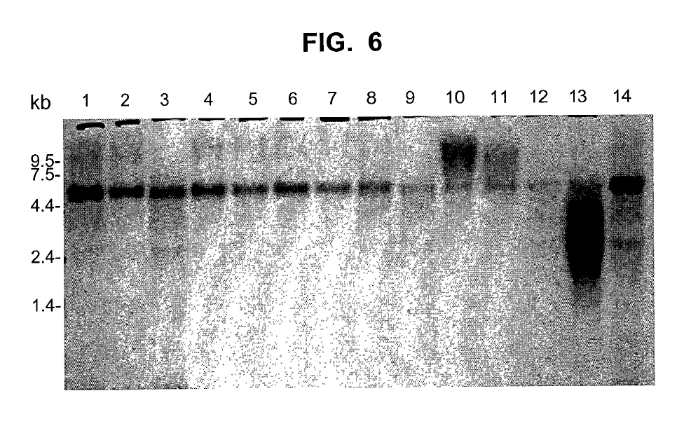

103P3E8 mRNA expression in normal human tissues was analyzed by Northern blotting of two multiple tissue blots (Clontech; Palo Alto, Calif.), comprising a total of 16 different normal human tissues, using labeled 103P3E8 SSH fragment (Example 1) as a probe. RNA samples were quantitatively normalized with a P-actin probe. The results demonstrated expression primarily in prostate, with significantly lower expression detected in testis, colon, pancreas, placenta, lung and kidney (FIG. 5). To analyze 103P3E8 expression in cancer tissues, northern blotting was performed on RNA derived from the LAPC xenografts, and several prostate and non-prostate cancer cell lines. The results show high expression levels of a 5-6 kb transcript in LAPC-4 AD, LAPC-4 AI, LAPC-9 AD, LAPC-9 AI and LAPC-3 AI when compared to normal prostate (FIG. 5C and FIG. 6).

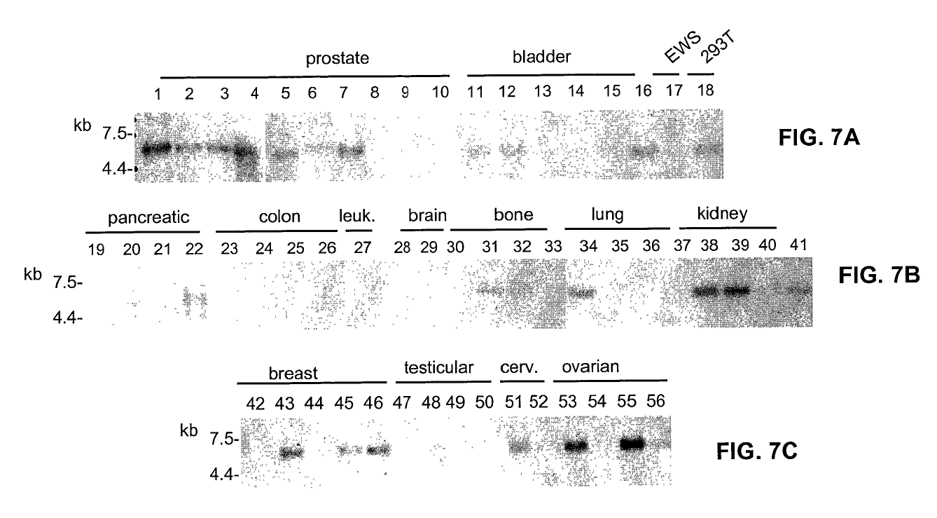

More detailed analysis of the xenografts shows that 103P3E8 is highly expressed in the xenografts even when grown within the tibia of mice (FIG. 6). Expression of 103P3E8 was also detected in several cancer cell lines derived from kidney (769-P, A498) and ovary (OV-1063, SW626) (FIG. 7). Lower expression levels were detected in multiple prostate (TSLPR1, LNCaP), bladder (HT1197, SCABER, 5637), pancraetic (Capan-1), bone (HOS, U2-OS), lung (CALU-1), kidney (SW839), breast (CAMA-1, MCF-7, MDA-MB-435s) and cervical (A431) cancer cell lines.

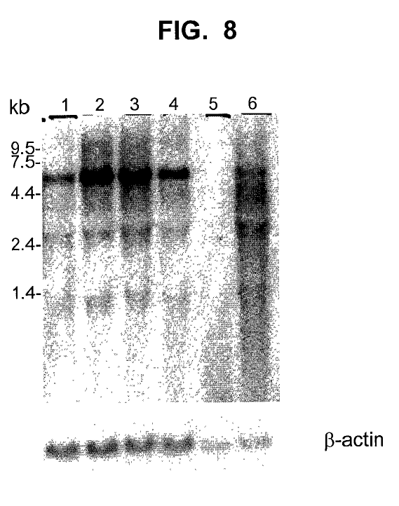

Northern analysis also shows that 103P3E8 is expressed in the normal prostate and prostate tumor tissues derived from prostate cancer patients (FIG. 8). In these cases, 2 out of 3 patients show higher expression in the tumor specimens compared to the normal adjacent tissue. It may be that expression of 103P3E8 increases as the tumor progresses to a more aggressive phenotype. For instance, the xenografts, which are derived from metastasized prostate cancer, show significantly more 103P3E8 expression compared to normal prostate (see FIG. 5). These results suggest that 103P3E8 is a prostate gene that is over expressed in prostate cancer and may have a functional role in prostate cancer pathology.

103P3E8 expression in normal tissues can be further analyzed using a multi-tissue RNA dot blot containing different samples (representing mainly normal tissues as well as a few cancer cell lines).

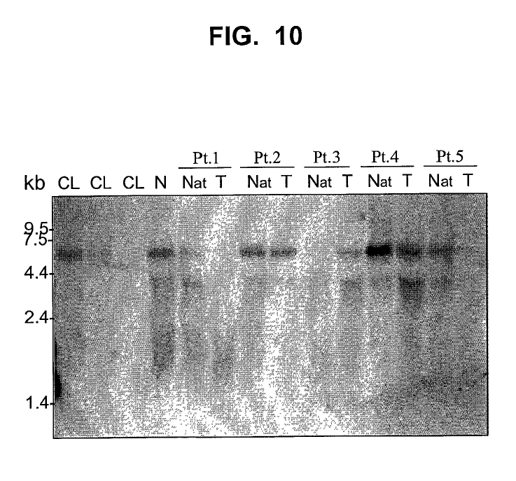

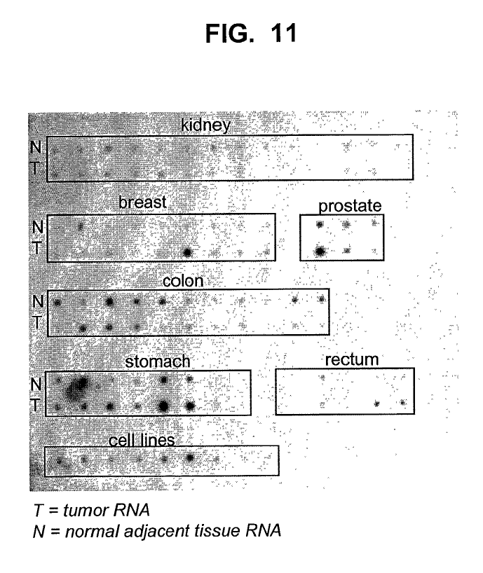

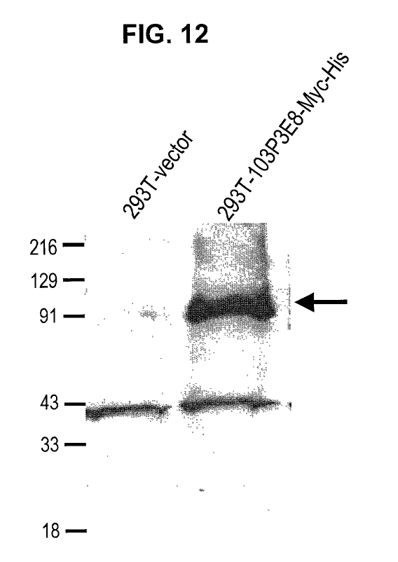

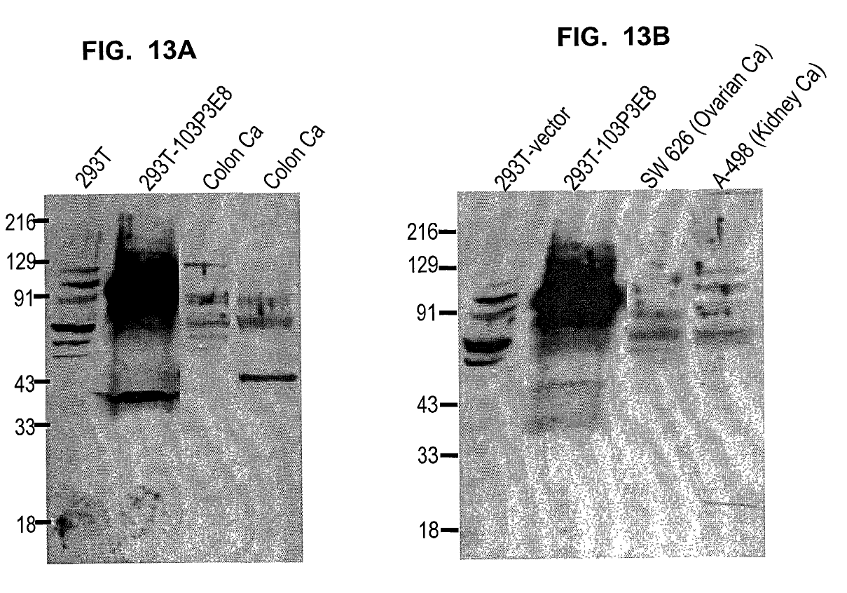

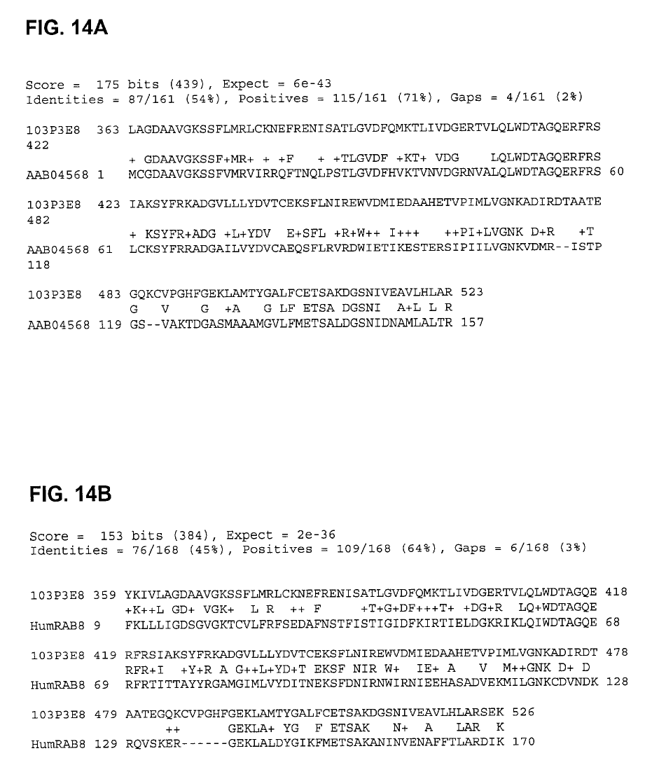

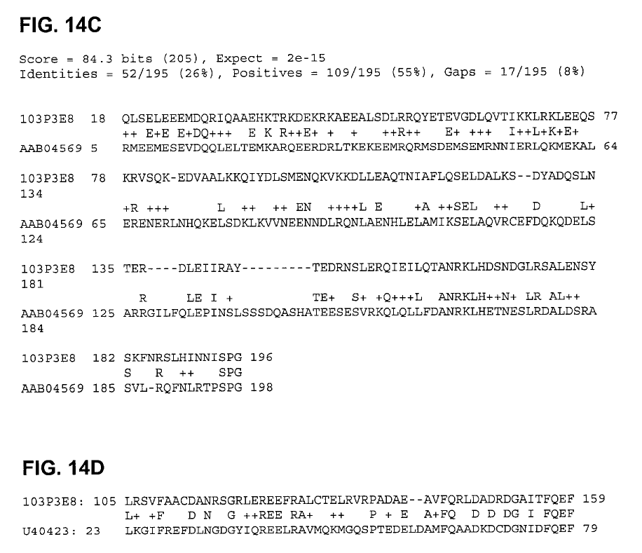

Analysis by RT-PCR showed expression of 103P3E8 in all tumors tested, prostate, bladder, kidney, colon, and lung (FIG. 9). Detailed Northern blot analysis shows that 103P3E8 is expressed in all colon tumor tissues derived from colon cancer patients (FIG. 10). It is also expressed in kidney, breast, prostate, colon, stomach and rectum patient cancer samples by dot-blot analysis (FIG. 11). The expression detected in normal adjacent tissues (isolated from diseased tissues) but not in normal tissues, isolated from healthy donors, indicate that these tissues are not fully normal and that 103P3E8 is expressed in early stage tumors, and thus can be used as a diagnostic target. Analysis of 9 human cancer cell lines showed highest expression of 103P3E8 in the lung carcinoma cell line A549 (FIG. 11)

### Example 5: Generation of 103P3E8 Polyclonal Antibodies

Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. For example, 103P3E8, recombinant bacterial fusion proteins or peptides encoding various regions of the 103P3E8 sequence are used to immunize New Zealand White rabbits. Typically a peptide can be designed from a coding region of 103P3E8. The peptide can be conjugated to keyhole limpet hemocyanin (KLH) and used to immunize a rabbit. Alternatively the immunizing agent may include all or portions of the 103P3E8 protein, analogs or fusion proteins thereof. For example, the 103P3E8 amino acid sequence can be fused to any one of a variety of fusion protein partners that are well known in the art, such as maltose binding protein, LacZ, thioredoxin or an immunoglobulin constant region (see e.g. Current Protocols In Molecular Biology, Volume 2, Unit 16, Frederick M. Ausubel et al. eds., 1995; Linsley, P. S., Brady, W., Urnes, M., Grosmaire, L., Damle, N., and Ledbetter, L.(1991) J Exp. Med. 174, 561-566). Other recombinant bacterial proteins include glutathione-S-transferase (GST), and HIS tagged fusion proteins of 103P3E8 that are purified from induced bacteria using the appropriate affinity matrix.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate).

In a typical protocol, rabbits are initially immunized subcutaneously with up to 200 μg, typically 50-200 μg, of fusion protein or peptide conjugated to KLH mixed in complete Freund's adjuvant. Rabbits are then injected subcutaneously every two weeks with up to 200 μg, typically 50-200 μg, of immunogen in incomplete Freund's adjuvant. Test bleeds are taken approximately 7-10 days following each immunization and used to monitor the titer of the antiserum by ELISA.

To test serum, such as rabbit serum, for reactivity with 103P3E8 proteins, the full-length 103P3E8 cDNA can be cloned into an expression vector such as one that provides a 6His tag at the carboxyl-terminus (pCDNA 3.1 myc-his, Invitrogen). After transfection of the constructs into 293T cells, cell lysates can be probed with anti-His antibody (Santa Cruz Biotechnologies, Santa Cruz, Calif.) and the anti-1 03P3E8 serum using Western blotting. Alternatively specificity of the antiserum is tested by Western blot and immunoprecipitation analyses using lysates of cells that express 103P3E8. Serum from rabbits immunized with GST or MBP fusion proteins is first semi-purified by removal of anti-GST or anti-MBP antibodies by passage over GST and MBP protein columns respectively. Sera from His-tagged protein and peptide immunized rabbits as well as depleted GST and MBP protein sera are purified by passage over an affinity column composed of the respective immunogen covalently coupled to Affigel matrix (BioRad).

Specifically, a GST-fusion protein consisting of amino acids 277-400 of the 103P3E8 coding sequence of FIG. 3 was used to immunize a New Zealand White rabbit. An initial injection of 200 μg of GST-fusion antigen in Freund's Complete Adjuvant (CFA) was given followed every 2 weeks thereafter by 200 μg injections in Freund's Incomplete Adjuvant (IFA). The antiserum generated was depleted of anti-GST-reactive antibodies by passage over a GST-affinity column and then affinity purified using the GST-fusion immunogen.

FIG. 12 shows expression of 103P3E8 protein in 293T cells with the recognition by the anti-103P3E8 polyclonal antibody. 293T cells were transiently transfected with either a pCDNA 3.1 Myc-His epitope tagged expression vector encoding 103P3E8 cDNA or with an empty control vector. Two days later, cells were harvested and lysed in SDS-PAGE sample buffer. Cell lysates (˜25 μg) were separated by SDS-PAGE and transferred to nitrocellulose. The membrane was blocked and then probed with an anti-103P3E8 affinity purified pAb raised to a GST-fusion protein encoding amino acids 277400 of the 103P3E8 coding sequence set forth in FIG. 3. Immunoreactive bands were detected by incubation with an HRP-conjugated anti-rabbit secondary antibody and visualized by enhanced chemiluminescence and exposure to autordiographic film. A predominant anti-103P3E8 immunoreactive band (arrow) was present in cell lysates from 293T-103P3E8 cells and a fainter band in cell lysates of control 293T cells indicating endogenous expression of 103P3E8 in these cells

As shown in FIG. 13 panels (A) and (13), colon, ovarian, and kidney cancer cells also express 103P3E8 protein. Lysates (˜25 μg/lane) from tumor tissue from colon cancer patients (panel A) or of the indicated ovarian and kidney cancer cell lines (panel B) were subjected to Western analysis using a rabbit anti-103P3E8 polyclonal antibody. Lysates of 293T cells transfected with either empty vector or with a 103P3E8 expression vector were included as positive controls. Anti-103P3E8 immunoreactive bands were developed by incubation with an anti-rabbit-HRP conjugated secondary antibody and visualized by enhanced chemiluminescence and exposure to autoradiographic film.

### Example 6: Generation of 103P3E8 Monoclonal Antibodies (mAbs)

In one embodiment, therapeutic mAbs to 103P3E8 will include those that react with epitopes of 103P3E8 involved in function activity. Immunogens for generation of such mAbs are designed to encode regions of the 103P3E8 protein predicted to have functional activity, for example, by domain homology or motif analysis (see, e.g., Tables V-XIX). These immunogens include peptides, recombinant bacterial proteins, and mammalian expressed Tag5 proteins and human and murine IgG FC fusion proteins. To generate mAbs to 103P3E8, mice are first immunized intraperitoneally (IP) with up to 100 μg, typically 5-50 μg, of protein immunogen mixed in complete Freund's adjuvant. Mice are then subsequently immunized IP every 2-4 weeks with up to 100 μg, typically 5-50 μg, of antigen mixed in Freund's incomplete adjuvant. Alternatively, Ribi adjuvant is used immunizations. In addition, a DNA-based immunization protocol is employed in which a mammalian expression vector encoding 103P3E8 sequence is used to immunize mice by direct injection of the plasmid DNA. For example, pCDNA 3.1 encoding either the full length 103P3E8 cDNA or extracellular coding regions of 103P3E8 fused to the coding sequence of murine or human IgG are used. This protocol is used alone or in combination with protein immunogens. Test bleeds are taken 7-10 days following immunization to monitor titer and specificity of the immune response. Once appropriate reactivity and specificity is obtained as determined by ELISA, Western blotting, and immunoprecipitation analyses, fusion and hybridoma generation is then carried with established procedures well known in the art (Harlow and Lane, 1988).

In one embodiment for generating 103P3E8 monoclonal antibodies, a glutathione-S-transferase (GST) fusion protein encompassing the carboxy-terminal domain of 103P3E8 (amino acids 277-400) is expressed, purified, and used as immunogen. Balb C mice are initially immunized intraperitoneally with 25 μg of the GST-103P3E8 fusion protein mixed in complete Freund's adjuvant. Mice are subsequently immunized every two weeks with 25 μg of GST-103P3E8 protein mixed in Freund's incomplete adjuvant for a total of three immunizations. To determine titer of serum from immunized mice, ELISA is carried out using a 103P3E8-specific cleavage fragment of the immunogen in which GST is removed by site specific proteolysis. Reactivity and specificity of serum to fall length 103P3E8 protein is monitored by Western blotting and flow cytometry using 293T cells transfected with an expression vector encoding the 103P3E8 cDNA (Example 7). Mice showing the strongest reactivity are rested for three weeks and given a final injection of 103P3E8 cleavage fragment in PBS and then sacrificed four days later. The spleens of the sacrificed mice are then harvested and fused to SPO/2 myeloma cells using standard procedures (Harlow and Lane, 1988). Supernatants from growth wells following HAT selection are screened by ELISA, Western blot, and flow cytometry to identify 103P3E8 specific antibody-producing clones.

The binding affinity of a 103P3E8 monoclonal antibody is determined using standard technologies. Affinity measurements quantify the strength of antibody to epitope binding and can be used to help define which 103P3E8 monoclonal antibodies are preferred for diagnostic or therapeutic use. The BIAcore system (Uppsala, Sweden) is a preferred method for determining binding affinity. The BIAcore system uses surface plasmon resonance (SPR, Welford K. 1991, Opt. Quant. Elect. 23: 1; Morton and Myszka, 1998, Methods in Enzymology 295: 268) to monitor biomolecular interactions in real time. BIAcore analysis conveniently generates association rate constants, dissociation rate constants, equilibrium dissociation constants, and affinity constants.

### Example 7: Production of Recombinant 103P3E8 in Bacterial and Mammalian Systems

### Bacterial Constructs

pGEX Constructs

To express 103P3E8 in bacterial cells, portions of 103P3E8 were fused to the Glutathione S-transferase (GST) gene by cloning into pGEX-4P-2 (Amersham Pharmacia Biotech, N.J.). The constructs were made in order to generate recombinant 103P3E8 protein sequences with GST fused at the N-terminus and a six histidine epitope at the C-terminus. The six histidine epitope tag is generated by adding the histidine codons to the cloning primer at the 3′ end of the open reading frame (ORF). A Thrombin recognition site permits cleavage of the GST tag from 103P3E8-related protein. The ampicillin resistance gene and pBR322 origin permits selection and maintenance of the plasmid in E. coli. For example, cDNA encoding the following fragment of 103P3E8 protein was cloned into pGEX4P-2: amino acids 180 to 798. In addition, nucleic acids that encode the following fragments are cloned into pGEX-4P-2: amino acids 277 to 470. In addition, nucleic acids that encode the following fragments are cloned into pGEX-4P-2: amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof.

pMAL Constructs

To express 103P3E8 in bacterial cells, all or part of the 103P3E8 nucleic acid sequence (such as amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof) are fused to the maltose-binding protein (MBP) gene by cloning into pMAL-c2X and pMAL-p2X (New England Biolabs, Mass.). The constructs are made to generate recombinant 103P3E8 protein sequences with MBP fused at the N-terminus and a six histidine epitope at the C-terminus. The six histidine epitope tag is generated by adding the histidine codons to the 3′ cloning primer. A Factor Xa recognition site permits cleavage of the GST tag from 103P3E8. The pMAL-c2X and pMAL-p2X vectors are optimized to express the recombinant protein in the cytoplasm or periplasm respectively. Periplasm expression enhances folding of proteins with disulfide bonds. For example, cDNA encoding the following fragments of 103P3E8 protein are cloned into pMAL: amino acids 1 to 179; amino acids 180 to 276; amino acids 277 to 470; 276 to 798; amino acids 471 to 832; amino acids 93 to 832, or any 8, 9, 10, 11, 12, 13, 14 or 15 contiguous amino acids from 103P3E8 or an analog thereof.

pCRII

To generate 103P3E8 sense and anti-sense riboprobes for RNA in situ investigations, a pCRII construct (Invitrogen, Carlsbad Calif.) is generated using cDNA sequence encoding amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof. The pCRII vector has Sp6 and T7 promoters flanking the insert to drive the production of 103P3E8 RNA riboprobes which will be used in RNA in situ hybridization experiments.

### Mammalian Constructs

To express recombinant 103P3E8, the full or partial length 103P3E8 cDNA (such as that encoding amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof) can be cloned into any one of a variety of expression vectors known in the art. The constructs can be transfected into any one of a wide variety of mammalian cells such as 293T cells. Transfected 293T cell lysates can be probed with the anti-103P3E8 polyclonal serum, described in Example 4 above, in a Western blot.

The 103P3E8 gene and cDNA fragments can also be subcloned into the retroviral expression vector pSRαMSVtkneo and used to establish 103P3E8-expressing cell lines as follows: The 103P3E8 coding sequence (from translation initiation ATG and Kozak translation start consensus sequence to the termination codons) is amplified by PCR using ds cDNA template from 103P3E8 cDNA. The PCR product is subcloned into pSRαMSVtkneo vector and transformed into DH5α competent cells. Colonies are picked to screen for clones with unique internal restriction sites on the cDNA. The positive clone is confirmed by sequencing of the cDNA insert. The retroviral vectors can thereafter be used for infection and generation of various cell lines using, for example, NIH 3T3, TsuPr1, 293 or rat-1 cells.

Additional illustrative mammalian and bacterial systems are discussed below.

pcDNA4/HisMax-TOPO Constructs

To express 103P3E8, or any portion thereof (such as amino acids I to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof), in mammalian cells, the 103P3E8 ORF is cloned into pcDNA4/HisMax-TOPO Version A (cat# K864-20, Invitrogen, Carlsbad, Calif.). Protein expression is driven from the cytomegalovirus (CMV) promoter and the SP 1 63 translational enhancer. The recombinant protein has Xpress™ and six histidine epitopes fused to the N-terinus to aid in detection and purification of the recombinant protein. The pcDNA4/HisMax-TOPO vector also contains the bovine growth hormone (BGH) polyadenylation signal and transcription termination sequence to enhance mRNA stability along with the SV40 origin for episomal replication and simple vector rescue in cell lines expressing the large T antigen. The Zeocin resistance gene allows for selection of mammalian cells expressing the protein and the ampicillin resistance gene and ColE1 origin permits selection and maintenance of the plasmid in E. coli.

pcDNA3.1/MycHis Constructs

To express 103P3E8 in mammalian cells, amino acids 276 to 798 with Kozak translation initiation site was cloned into pcDNA3.1/MycHis Version A (Invitrogen, Carlsbad, Calif.). An analogous protocol is followed for any portion of 103P3E8, e.g., amino acids I to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof, along with Kozak sequences is cloned into pcDNA3.1/MycHis_Version A. Protein expression is driven from the cytomegalovirus (CMV) promoter. The recombinant protein has the myc epitope and six histidines fused to the C-terminus to aid in detection and purification of the recombinant protein. The pcDNA3.1/MycHis vector also contains the bovine growth hormone (BGH) polyadenylation signal and transcription termination sequence to enhance mRNA stability, along with the SV40 origin for episomal replication and simple vector rescue in cell lines expressing the large T antigen. The Neomycin resistance gene can be used, as it allows for selection of mammalian cells expressing the protein and the ampicillin resistance gene and ColE1 origin permits selection and maintenance of the plasmid in E. coli.

pcDNA3.1/V5His-TOPO Constructs

To express 103P3E8 in mammalian cells, the cDNA encoding the 103P3E8 ORF, or amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof along with Kozak consensus translation initiation sequence are cloned into pcDNA4NV5His-TOPO (cat# K4800-01, Invitrogen, Carlsbad, Calif.). Protein expression is driven from the cytomegalovirus (CMV) promoter. The recombinant protein has V5™ and six histidine epitopes fused at the C-terminus to aid in detection and purification of the recombinant protein. The pcDNA4/V5His-TOPO vector also contains the bovine growth hormone (BGH) polyadenylation signal and transcription termination sequence to enhance mRNA stability along with the SV40 origin for episomal replication and simple vector rescue in cell lines expressing the large T antigen. The Zeocin resistance gene allows for selection of mammalian cells expressing the protein and the ampicillin resistance gene and ColE1 origin permits selection and maintenance of the plasmid in E. coli.

pcDNA3.1CT-GFP-TOPO Constructs

To express 103P3E8 in mammalian cells and to allow detection of the recombinant protein using fluorescence, the ORF, or amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof along with consensus Kozak translation initiation site are cloned into pcDNA3.1CT-GFP-TOPO (Invitrogen, Calif.). Protein expression is driven from the cytomegalovirus (CMV) promoter. The recombinant protein has the Green Fluorescent Protein (GFP) fused to the C-terminus facilitating non-invasive, in vivo detection and cell biology studies. The pcDNA3.1/MycHis vector also contains the bovine growth hormone (BGH) polyadenylation signal and transcription termination sequence to enhance mRNA stability along with the SV40 origin for episomal replication and simple vector rescue in cell lines expressing the large T antigen. The Neomycin resistance gene allows for selection of mammalian cells that express the protein, and the ampicillin resistance gene and ColE1 origin permits selection and maintenance of the plasmid in E. coli. An additional construct with a N-terminal GFP fusion is made in pcDNA3.1NT-GFP-TOPO spanning the entire length of the 103P3E8 protein.

pAPtag Constructs

The cDNA encoding 103P3E8 amino acids 179 to 798, 1 to 178, 180 to 360, 360 to 798, 1 to 832; or any 8, 9, 10, 11, 12, 13, 14 or 15 contiguous amino acids from 103P3E8 or an analog thereof are cloned into pAPtag-5 (GenHunter Corp. Nashville, Tenn.). This construct generates an alkaline phosphatase fusion at the C-terminus of the 103P3E8 protein while fusing the IgGK signal sequence to N-terminus. The resulting recombinant 103P3E8 protein is optimized for secretion into the media of transfected mammalian cells and can be used to identify proteins such as ligands or receptors that interact with the 103P3E8 protein. Protein expression is driven from the CMV promoter and the recombinant protein also contains myc and six histidines fused to the C-terminus of alkaline phosphatase to aid in detection and purification of the recombinant protein. The Zeosin resistance gene allows for selection of mammalian cells expressing the protein and the ampicillin resistance gene permits selection of the plasmid in E. coli.

ptag5 Constructs

The cDNA encoding for 103P3E8 amino acids amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof are cloned into pTag-5. This vector is similar to pAPtag but without the alkaline phosphatase fusion. This construct generates an immunoglobulin GI Fc fusion at the C-terminus of the 103P3E8 protein while fusing the IgGK signal sequence to the N-terminus. The resulting recombinant 103P3E8 protein is optimized for secretion into the media of transfected mammalian cells, and can be used to identify proteins such as ligands or receptors that interact with the 103P3E8 protein. Protein expression is driven from the CMV promoter and the recombinant protein also contains myc and six histidines fused to the C-terminus to aid in detection and purification of the recombinant protein. The Zeocin resistance gene allows for selection of mammalian cells expressing the protein, and the ampicillin resistance gene permits selection of the plasmid in E. coli.

psecFc Constructs

The cDNA encoding for 103P3E8 amino acids amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof are cloned into psecFc. The psecFc vector was assembled by cloning immunoglobulin G1 Fc (hinge, CH2, CH3 regions) into pSecTag2 (Invitrogen, Calif.). This construct generates an immunoglobulin G1 Fc fusion at the C-terminus of the 103P3E8 protein, while fusing the IgGK signal sequence to N-terminus. The resulting recombinant 103P3E8 protein is optimized for secretion into the media of transfected mammalian cells, and can be used to identify proteins such as ligands or receptors that interact with the 103P3E8 protein. Protein expression is driven from the CMV promoter. The Zeocin resistance gene allows for selection of mammalian cells that express the protein, and the ampicillin resistance gene permits selection of the plasmid in E. coli.

pSRα Constructs

To generate mammalian cell lines that express 103P3E8 constitutively, cDNA coding for amino acids amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof are cloned into pSRα constructs along with a Kozak translation initiation sequence. Amphotropic and ecotropic retroviruses were generated by transfection of pSRα constructs into the 293T-10A1 packaging line or co-transfection of pSRα and a helper plasmid (φ˜) in the 293 cells, respectively. The retrovirus was used to infect a variety of mammalian cell lines, resulting in the integration of the cloned gene, 103P3E8, into the host cell-lines. Protein expression is driven from a long terminal repeat (LTR). The Neomycin resistance gene allows for selection of mammalian cells that express the protein, and the ampicillin resistance gene and ColE1 origin permit selection and maintenance of the plasmid in E. coli.

Additional pSRα constructs were made that fused the FLAG tag to the C-terminus and N-terminus to allow detection using anti-FLAG antibodies. The FLAG sequence 5′ gat tac aag gat gac gac gat aag 3′ (SEQ ID NO: 6) were added to cloning primer at the 5′ and 3′ ends of the ORF.

Additional pSRα constructs are made to produce both N-terminal and C-terminal GFP and myc/6 HIS fusion proteins of the full-length 103P3E8 protein.

### Example 8: Production of Recombinant 103P3E8 in a Baculovirus System To generate a recombinant 103P3E8 protein in a baculovirus expression system, cDNA sequence encoding the 103P3E8 protein or amino acids 1 to 179; amino acids 180 to 276; 276 to 798; amino acids 471 to 832; amino acids 93 to 832, or any 8, 9, 10, 11, 12, 13, 14 or 15 contiguous amino acids from 103P3E8 or an analog thereof are cloned into the baculovirus transfer vector pBlueBac 4.5 (Invitrogen), which provides a His-tag at the N-terminus. Specifically, pBlueBac-103P3E8 is co-transfected with helper plasmid pBac-N-Blue (Invitrogen) into SF9 (Spodoptera frugiperda) insect cells to generate recombinant baculovirus (see Invitrogen instruction manual for details). Baculovirus is then collected from cell supernatant and purified by plaque assay.

Recombinant 103P3E8 protein is then generated by infection of HighFive insect cells (Invitrogen) with the purified baculovirus. Recombinant 103P3E8 protein can be detected using anti-103P3E8 antibody. 103P3E8 protein can be purified and used in various cell-based assays or as immunogen to generate polyclonal and monoclonal antibodies specific for 103P3E8.

### Example 9: 103P3E8 Homology Comparison to Known Sequences

The 103P3E8 cDNA clone encodes a 625 amino acid ORF (5′ open) predicted to be localized to the nucleus based on PSORT analysis (http://psort.nibb.ac.jp:8800/form.html) and the presence of a leucine zipper.

Nucleotide sequence analysis of the 103P3E8 carboxyl-terminal region reveals highest homology to a ras-like GTP-binding protein in C. elegans (Accession No. AAB04568; Wilson et al., 1994, Nature 368:32-38). The two protein sequences are 54% identical and 71% homologous over a 161 amino acid region (FIG. 14). The closest human homolog is RAB8, also known as the c-mel oncogene (Nimmo et al., 1991, Oncogene 6:1347-1351; Zahraoui et al., 1994, J. Cell Biol. 124:101-115), with 45% identity and 64% homology over a 168 amino acid region (FIG. 14). Analysis of the amino-terminal region reveals similarity to a putative intermediate filament protein from C. elegans (Accession No. AAB04569; Wilson et al., 1994, Nature 368:32-38; FIG. 14), and weaker homology to the potential coiled coil region of human smooth muscle myosin heavy chain (Accession No. P35749; Matsuoka et al., 1993, Am. J. Med. Genet. 46:61-67).

The 103P3E8 clone 7 protein is encompassed within a protein sequence (FIG. 3, underlined region). The clone 7 protein is truncated at the C-terminus which could represent the cancer form of the protein since it was cloned from an LAPC4AD library as opposed to a normal prostate library. The 103P3E8 protein has a myosin tail domain at amino acids 266 to 467 and Rab domain at amino acids 634 to 800 of the protein in FIG. 3. The protein sequence shows homology to p21ras-like G proteins at the carboxyl-terminus and some homology to intermediate filament proteins closer to the amino-terminus.

Based on amino acid sequence, 103P3E8 demonstrates significant homology to three classes of proteins. At it amino terminus (aa 1-170), 103P3E8 is homologous to an EF-Hand calcium binding protein with serine/threonine kinase activity (Identity 42%, Homology 53% to U40423). At its C-terminus (aa 400-832), 103P3E8 shows homology to small GTP-binding proteins (Identities 43%, Homology 62% with P20790), including the Ras-related RAB8 protein (identity 43%, homology 59% with Z73946). The middle section of 103P3E8 (aa 200-500) exhibits homology to myosin heavy chain (identity 42%, homology 57% with U40423) and to a cytoplasmic link protein named CLIP-170 (identity 35%, homology 59% with AF030879).

Proteins containing EF-hand motifs are calcium-binding proteins that participate in cell signaling. Ras-like GTP binding proteins, including Rab, play a role in vesicle trafficking, cell signaling, cell growth, and motility. While myosin functions as an intermediate filament and regulates contraction, CLIP-170 associates with intermediate filaments and links endocytic vesicles to microtubules.

Taken together, this information suggests that 103P3E8 can function in a manner similar to the ras oncogene and direct the activation of key signaling cascades involved in tumor growth and progression. It is possible that cellular signaling by 103P3E8 is regulated by calcium as suggested by the presence of an EF-hand domain. Another possible function of 103P3E8 is a role in cellular trafficking and cell motility, especially as several EF-containing proteins as well as CLIP 170 have been shown to regulate vesicle endocytosis (de Beer T et al. Nat Struct Biol. 2000, 7:1018; Pierre P. Cell. 1992, 70:887). Based on the PSORT prediction that 103P3E8 is a nuclear protein, it is also probable that 103P3E8 participates in regulating gene expression.

### Example 10: Identification of Potential Signal Transduction Pathways

Small GTP-binding proteins have been reported to interact with a variety of signaling molecules and regulate signaling pathways (Reuther G. W., Der C. J. Curr Opin Cell Biol. 2000 12:157). The characteristics of Ras-like G proteins makes them excellent targets for anti-neoplastic drug therapy (Scharovsky O. G. et al. J Biomed Sci. 2000. 7:292). Using immunoprecipitation and Western blotting techniques, proteins that associate with 103P3E8 and mediate signaling events are identified. Several pathways known to play a role in cancer biology can be regulated by 103P3E8, including phospholipid pathways such as PI3K, AKT, etc, adhesion and migration pathways, including FAK, Rho, Rac-1, etc as well as mitogenic/survival cascades such as ERK, p38, etc (Cell Growth Differ. 2000,11:279; J Biol Chem. 1999, 274:801; J. Cell Biol. 1997, 138:913; Oncogene. 2000, 19:3003). Using Western blotting techniques, one can evaluate the role that 103P3E8 plays in the regulation of these pathways.

Cells lacking 103P3E8 and cells expressing 103P3E8 are either left untreated or stimulated with cytokines, androgen and anti-integrin antibodies. Cell lysates are analyzed using anti-phosphos-specific antibodies (Cell Signaling, Santa Cruz Biotechnology) in order to detect phosphorylation and regulation of ERK, p38, AKT, PI3K, PLC and other signaling molecules. Using the same Western blotting approach, one can determine when proteins, small molecules or antibodies generated against 103P3E8 have the ability to modulate the activity of one or more signaling pathways. When 103P3E8 plays a role in the regulation of signaling pathways, 103P3E8 is used as a target for diagnostic, preventative and therapeutic purposes.

To determine whether 103P3E8 directly or indirectly activates known signal transduction pathways in cells, luciferase (luc)-based transcriptional reporter assays are carried out in cells that express 103P3E8. These transcriptional reporters contain consensus-binding sites for known transcription factors that lie downstream of well-characterized signal transduction pathways. The reporters and examples of these associated transcription factors, signal transduction pathways, and activation stimuli are listed below.

1. NFkB-luc, NFkB/Rel; Ik-kinase/SAPK; growth/apoptosis/stress

2. SRE-luc, SRF/TCF/ELK1; MAPK/SAPK; growth/differentiation

3. AP-1-luc, FOS/JUN; MAPK/SAPK/PKC; growth/apoptosis/stress

4. ARE-luc, androgen receptor; steroids/MAPK; growth/differentiation/apoptosis

5. p53-luc, p53; SAPK; growth/differentiation/apoptosis

6. CRE-luc, CREB/ATF2; PKA/p38; growth/apoptosis/stress

103P3E8-mediated effects are assayed in cells showing mRNA expression. Luciferase reporter plasmids are introduced, e.g., by lipid-mediated transfection (TFX-50, Promega). Luciferase activity, an indicator of relative transcriptional activity, is measured by incubation of cell extracts with luciferin substrate and luminescence of the reaction is monitored in a luminometer.

Signaling pathways activated by 103P3E8 are mapped and used for the identification and validation of therapeutic targets in these pathways. When 103P3E8 is involved in cell signaling, it is used as a target for diagnostic, preventative and therapeutic purposes.

### Example 11: Involvement of 103P3E8 in Tumor Progression

103P3E8 contributes to the growth of cancer cells, whether by acting as a Ras-like G protein, regulating cell fate or by acting on intermediate filaments. The role of 103P3E8 in tumor growth is investigated in prostate, colon and kidney cell lines as well as NIH 3T3 cells engineered to stably express 103P3E8. Parental 103P3E8 negative cells and 103P3E8-expressing cells are evaluated for cell growth using a well-documented proliferation assay (Fraser S. P., Grimes J. A., Djamgoz M. B. Prostate. 2000;44:61, Johnson D. E., Ochieng J., Evans S. L. Anticancer Drugs. 1996, 7:288).

To determine the role of 103P3E8 in the transformation process, its effect in colony forming assays is investigated by techniques known in the art. For example, parental NIH3T3 cells lacking 103P3E8 are compared to NHI-3T3-103P3E8 cells in a soft agar assay under stringent and more permissive conditions (see e.g., Song Z. et al. Cancer Res. 2000;60:6730).

To determine the role of 103P3E8 in invasion and metastasis of cancer cells, a well-established Transwell Insert System assay (Becton Dickinson) is used (see, e.g., Cancer Res . 1999; 59:6010). Cells lacking 103P3E8 and cells expressing 103P3E8 are loaded with the fluorescent dye, calcein, and plated in the top well of the Transwell insert. Invasion is determined by fluorescence of cells in the lower chamber relative to the fluorescence of the entire cell population.

103P3E8 also plays a role in cell cycle and apoptosis. Parental and 103P3E8-expressing prostate, kidney, bladder, colon and lung cells are compared for differences in cell cycle regulation using a well-established BrdU assay (see, e.g., Abdel-Malek ZA. J Cell Physiol. 1988, 136:247). In short, cells are grown under both optimal (full serum) and limiting (low serum) conditions, are labeled with BrdU and stained with anti-BrdU Ab and propidium iodide. Cells are analyzed for entry into the G1, S, and G2-M phases of the cell cycle. Alternatively, the effect of stress on apoptosis is evaluated in 103P3E8-negative cells and 103P3E8-expressing cells, including normal and tumor prostate, bladder, kidney, colon and lung cells. Engineered and parental cells treated with various chemotherapeutic agents, such as doxorubicin, etoposide, etc, and protein synthesis inhibitors, such as cycloheximide etc, are stained with annexin V-FITC. Cell death is measured by FACS analysis.

When 103P3E8 plays a role in cell growth, transformation, invasion or apoptosis, it is used as a target for diagnostic, preventative and therapeutic purposes.

### Example 12: Analysis of 103P3E8 Expression in Subcellular Fractions

The cellular location of 103P3E8 proteins is assessed using subcellular fractionation techniques widely used in cellular biology (see, e.g., Storrie B, et al. Methods Enzymol. 1990;182:203-25). Prostate, kidney, colon, bladder or other cell lines are separated into nuclear, cytosolic and membrane fractions. The expression of 103P3E8 in the different fractions is tested using Western blotting techniques.

Alternatively, to determine the subcellular localization of 103P3E8, 293T cells are transfected with an expression vector encoding His-tagged 103P3E8 or Flag-tagged 103P3E8 (PCDNA 3.1 MYC/HIS, Invitrogen, Flag-pSRa). The transfected cells are harvested and subjected to a differential subcellular fractionation protocol as previously described (Pemberton, P. A. et al, 1997, J of Histochemistry and Cytochemistry, 45:1697-1706.) This protocol separates the cell into fractions enriched for nuclei, heavy membranes (lysosomes, peroxisomes, and mitochondria), light membranes (plasma membrane and endoplasmic reticulum), and soluble proteins.

Cellular localization is also determined by fluorescent microscopy methods. Green Fluorescent Protein (GFP) tagged 103P3E8 is expressed in a variety of cell lines. Cells are placed on slides and examined by fluorescent microscopy for the location of the GFP-103P3E8.

### Example 13: Function of 103P3E8 as a Small GTP Binding Protein

Ras-like G binding proteins have been shown to cycle between a GDP inactive form and a GTP active state (Trahey M, McCormick F. Science 1987, 238:542). In order to determine whether 103P3E8 binds GTP and therefore acquires an active G protein form, cells expressing either untagged or HIS-tagged 103P3E8 are labeled with ³²P-orthophosphate. Cells are lysed, and 103P3E8 is immunoprecipitated using anti-103P3E8 antibody or anti-HIS tag antibody. The nucleotides are eluted from the immunoprecipitates and evaluated for GTP and GDP content by separating them on a TLC plate (Bhullar RP. Biochem Biophys Acta. 1996, 1311:181). Comparison of cells lacking 103P3E8 and cells expressing 103P3E8 reveals the endogenous level of 103P3E8 activation. Comparison of resting untreated 103P3E8 expressing cells and cells treated with phorbol ester, TPA, cytokines or antibodies to surface proteins indicates the level of 103P3E8 activation in stimulated growing cells. Using this and analogous art-accepted assay systems, it is determined whether antibodies to 103P3E8 alter its activation state. When 103P3E8 functions as a small GTP binding protein, it is used as a target for diagnostic, preventative and therapeutic purposes.

Due to the importance of GTP binding proteins in cell function as well as tumor growth and progression, G-proteins have become the focus of drug development efforts. Several approaches for targeting Ras-like proteins can be used. One approach is to alter the localization of the GTP binding protein and prevent it association with effector molecules. Such an approach is exemplified by farnesyl transferase inhibitors used to inhibit the effect of Ras (Curl, M. Anticancer Drugs 2001, 12:163; End, W. Cancer Res. 2001, 61:131). Inhibitors that prevent Ras interaction with immediate downstream effectors such as Raf-1 have also been designed (Zeng J. Protein Eng. 2001, 14:39). Inhibitors designed against Ras-like proteins and other G-proteins including 103P3E8 are used to modulate 103P3E8 function.

### Example 14: Involvement of 103P3E8 in Cell Communication

Small G proteins have been shown to participate in cell communication by regulating cell adhesion and adherens junctions (Akhtar N. Mol. Cell. Biol. 2001, 12:847). Using cells that express or lack 103P3E8, it is determined whether expression of 103P3E8 modifies cell-cell adhesion and matrix mediated adhesion. Cells expressing or lacking 103P3E8 are compared for their ability to form cell clusters. In addition, cells are compared for their ability to adhere to extracellular matrix and to other cell populations using techniques previously described (see, e.g., Haier et al, Br. J. Cancer. 1999, 80:1867; Lehr and Pienta, J. Natl. Cancer Inst. 1998, 90:118). Briefly, in one embodiment, cells labeled with a fluorescent indicator, such as calcein, are incubated on tissue culture wells coated with media alone or with matrix proteins. Adherent cells are detected by fluorimetric analysis and percent adhesion is calculated. This experimental system is used to identify proteins, antibodies and/or small molecules that modulate cell adhesion to extracellular matrix and cell-cell interaction. Since cell adhesion plays a critical role in tumor growth, progression, and, colonization, a gene involved in this process can serves as a diagnostic, preventative and therapeutic modality. When 103P3E8 functions in cell-cell communication, it is used as a target for diagnostic, preventative and therapeutic purposes

### Example 15: Regulation of Transcription by 103P3E8

The 103P3E8 protein can play a role in transcriptional regulation of eukaryotic genes. Regulation of gene expression is evaluated by studying gene expression in cells expressing or lacking 103P3E8. For this purpose, two types of experiments are performed. In the first set of experiments, RNA from parental and 103P3E8-expressing cells including NIH 3T3, prostate, bladder, kidney, colon and lung cell lines, are extracted and hybridized to commercially available gene arrays (Clontech). Resting cells as well as cells treated with FBS, cytokines or androgen are compared. Differentially expressed genes are identified in accordance with procedures known in the art. The differentially expressed genes are then mapped to biological pathways.

In the second set of experiments, specific transcriptional pathway activation is evaluated using commercially available (Stratagene) luciferase reporter constructs including: NFkB-luc, SRE-luc, ELK1-luc, ARE-luc, p53-luc, and CRE-luc. These transcriptional reporters contain consensus binding sites for known transcription factors that lie downstream of well-characterized signal transduction pathways, and represent a good tools to ascertain pathway activation and screen for positive and negative modulators of pathway activation. When 103P3E8 plays a role in gene regulation, 103P3E8 is used as a target for diagnostic, prognostic, preventative and therapeutic purposes.

### Example 16: In Vivo Assay for 103P3E8 Tumor Growth Promotion

The effect of the 103P3E8 protein on tumor cell growth can be evaluated in vivo by gene overexpression in tumor-bearing mice. For example, SCID mice can be injected SQ on each flank with 1×10⁶ of either PC3, TSUPR1, or DU145 cells containing tkNeo empty vector or 103P3E8. At least two strategies may be used: (1) Constitutive 103P3E8 expression under regulation of a promoter such as a constitutive promoter obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), or from heterologous mammalian promoters, e.g., the actin promoter or an immunoglobulin promoter, provided such promoters are compatible with the host cell systems. (2) Regulated expression under control of an inducible vector system, such as ecdysone, tet, etc., can be used provided such promoters are compatible with the host cell systems. Tumor volume is then monitored at the appearance of palpable tumors and is followed over time to determine if 103P3E8-expressing cells grow at a faster rate and whether tumors produced by 103P3E8-expressing cells demonstrate characteristics of altered aggressiveness (e.g. enhanced metastasis, vascularization, reduced responsiveness to chemotherapeutic drugs). Additionally, mice can be implanted with 1×10⁵ of the same cells orthotopically to determine if 103P3E8 has an effect on local growth in the prostate or on the ability of the cells to metastasize, specifically to lungs, lymph nodes, and bone marrow. Also see Saffran et al, “Anti-PSCA mAbs inhibit tumor growth and metastasis formation and prolong the survival of mice bearing human prostate cancer xenografts” PNAS 10:1073-1078 or www.pnas.org/cgi/doi/10.1073/pnas.051624698.

The assay is also useful to determine the 103P3E8 inhibitory effect of candidate therapeutic compositions, such as for example, 103P3E8 intrabodies, 103P3E8 antisense molecules and ribozymes.

### Example 17: 103P3E8 Monoclonal Antibody-mediated Inhibition of Prostate Tumors In Vivo

The significant expression of 103P3E8, in cancer tissues, together with its restrictive expression in normal tissues along with its expected cell surface expression makes 103P3E8 an excellent target for antibody therapy. Similarly, 103P3E8 is a target for T cell-based immunotherapy. Thus, the therapeutic efficacy of anti-103P3E8 mAbs in human prostate cancer xenograft mouse models is evaluated by using androgen-independent LAPC-4 and LAPC-9 xenografts (Craft, N., et al.,. Cancer Res, 1999. 59(19): p. 5030-6) and the androgen independent recombinant cell line PC3-103P3E8 (see, e.g., Kaighn, M. E., et al, Invest Urol, 1979. 17(1): p. 16-23).

Antibody efficacy on tumor growth and metastasis formation is studied, e.g., in a mouse orthotopic prostate cancer xenograft model. The antibodies can be unconjugated, as discussed in this Example, or can be conjugated to a therapeutic modality, as appreciated in the art. Anti-103P3E8 mAbs inhibit formation of both the androgen-dependent LAPC-9 and androgen-independent PC3-103P3E8 tumor xenografts. Anti-103P3E8 mAbs also retard the growth of established orthotopic tumors and prolonged survival of tumor-bearing mice. These results indicate the utility of anti-103P3E8 mAbs in the treatment of local and advanced stages of prostate cancer. (See, e.g., (Saffran, D., et al., PNAS 10: 1073-1078 or www.pnas.org/cgi/doi/10.1073/pnas.051624698)

Administration of the anti-103P3E8 mAbs led to retardation of established orthotopic tumor growth and inhibition of metastasis to distant sites, resulting in a significant prolongation in the survival of tumor-bearing mice. These studies indicate that 103P3E8 as an attractive target for immunotherapy and demonstrate the therapeutic potential of anti-103P3E8 mAbs for the treatment of local and metastatic prostate cancer. This example demonstrates that unconjugated 103P3E8 monoclonal antibodies are effective to inhibit the growth of human prostate tumor xenografts grown in SCID mice; accordingly a combination of such efficacious monoclonal antibodies is also effective.

### Tumor inhibition using multiple unconjugated 103P3E8 mAbs

### Materials and Methods

103P3E8 Monoclonal Antibodies:

Monoclonal antibodies are raised against 103P3E8 as described in Example 6. The antibodies are characterized by ELISA, Western blot, FACS, and immunoprecipitation for their capacity to bind 103P3E8. Epitope mapping data for the anti-103P3E8 mAbs, as determined by ELISA and Western analysis, recognize epitopes on the 103P3E8 protein. Immunohistochemical analysis of prostate cancer tissues and cells with these antibodies is performed.

The monoclonal antibodies are purified from ascites or hybridoma tissue culture supernatants by Protein-G Sepharose chromatography, dialyzed against PBS, filter sterilized, and stored at −20° C. Protein determinations are performed by a Bradford assay (Bio-Rad, Hercules, Calif.). A therapeutic monoclonal antibody or a cocktail comprising a mixture of individual monoclonal antibodies is prepared and used for the treatment of mice receiving subcutaneous or orthotopic injections of LAPC-9 prostate tumor xenografts.

Prostate Cancer Xenografts and Cell Lines

The LAPC-9 xenograft, which expresses a wild-type androgen receptor and produces prostate-specific antigen (PSA), is passaged in 6- to 8-week-old male ICR-severe combined immunodeficient (SCID) mice (Taconic Farms) by s.c. trocar implant (Craft, N., et al., supra). Single-cell suspensions of LAPC-9 tumor cells are prepared as described in Craft, et al. The prostate carcinoma cell line PC3 (American Type Culture Collection) is maintained in DMEM supplemented with L-glutamine and 10% (vol/vol) FBS.

A PC3-103P3E8 cell population is generated by retroviral gene transfer as described in Hubert, R. S., et al., STEAP: a prostate-specific cell-surface antigen highly expressed in human prostate tumors. Proc Natl Acad Sci U S A, 1999. 96(25): p. 14523-8. Anti-103P3E8 staining is detected by using an FITC-conjugated goat anti-mouse antibody (Southern Biotechnology Associates) followed by analysis on a Coulter Epics-XL flow cytometer.

Xenograft Mouse Models.

Subcutaneous (s.c.) tumors are generated by injection of 1×10⁶ LAPC-9, PC3, or PC3-103P3E8 cells mixed at a 1:1 dilution with Matrigel (Collaborative Research) in the right flank of male SCID mice. To test antibody efficacy on tumor formation, i.p. antibody injections are started on the same day as tumor-cell injections. As a control, mice are injected with either purified mouse IgG (ICN) or PBS; or a purified monoclonal antibody that recognizes an irrelevant antigen not expressed in human cells. In preliminary studies, no difference is found between mouse IgG or PBS on tumor growth. Tumor sizes are determined by vernier caliper measurements, and the tumor volume is calculated as length×width×height. Mice with s.c. tumors greater than 1.5 cm in diameter are sacrificed. PSA levels are determined by using a PSA ELISA kit (Anogen, Mississauga, Ontario). Circulating levels of anti-103P3E8 mAbs are determined by a capture ELISA kit (Bethyl Laboratories, Montgomery, Tex.). (See, e.g., (Saffran, D., et al., PNAS 10:1073-1078 or www.pnas.org/cgi/ doi/10.1073/pnas.051624698)

Orthotopic injections are performed under anesthesia by using ketamine/xylazine. An incision is made through the abdominal muscles to expose the bladder and seminal vesicles, which then are delivered through the incision to expose the dorsal prostate. LAPC-9 cells (5×10⁵) mixed with Matrigel are injected into each dorsal lobe in a 10-μl volume. To monitor tumor growth, mice are bled on a weekly basis for determination of PSA levels. Based on the PSA levels, the mice are segregated into groups for the appropriate treatments. To test the effect of anti-103P3E8 mAbs on established orthotopic tumors, i.p. antibody injections are started when PSA levels reach 2-80 ng/ml.

Anti-103P3E8 mAbs Inhibit Growth of 103P3E8-Expressing Prostate-Cancer Tumors

The effect of anti-103P3E8 mAbs on tumor formation is tested by using the LAPC-9 orthotopic model. As compared with the s.c. tumor model, the orthotopic model, which requires injection of tumor cells directly in the mouse prostate, results in a local tumor growth, development of metastasis in distal sites, deterioration of mouse health, and subsequent death (Saffran, D., et al., PNAS supra; Fu, X., et al., Int J Cancer, 1992. 52(6): p. 987-90; Kubota, T., J Cell Biochem, 1994. 56(1): p. 4-8). The features make the orthotopic model more representative of human disease progression and allowed us to follow the therapeutic effect of mAbs on clinically relevant end points.

Accordingly, LAPC-9 tumor cells are injected into the mouse prostate, and 2 days later, the mice are segregated into two groups and treated with either up to 200μg, usually 10-50μg, of anti-103P3E8 Ab or PBS three times per week for two to five weeks. Mice are monitored weekly for circulating PSA levels as an indicator of tumor growth.

A major advantage of the orthotopic prostate-cancer model is the ability to study the development of metastases. Formation of metastasis in mice bearing established orthotopic tumors is studies by IHC analysis on lung sections using an antibody against a prostate-specific cell-surface protein STEAP expressed at high levels in LAPC-9 xenografts (Hubert, R. S., et al., Proc Natl Acad Sci U S A, 1999. 96(25): p. 14523-8).

Mice bearing established orthotopic LAPC-9 tumors are administered 11 injections of either anti-103P3E8 mAb or PBS over a 4-week period. Mice in both groups are allowed to establish a high tumor burden (PSA levels greater than 300 ng/ml), to ensure a high frequency of metastasis formation in mouse lungs. Mice then are killed and their prostate and lungs are analyzed for the presence of LAPC-9 cells by anti-STEAP IHC analysis.

These studies demonstrate a broad anti-tumor efficacy of anti-103P3E8 antibodies on initiation and progression of prostate cancer in xenograft mouse models. Anti-103P3E8 antibodies inhibit tumor formation of both androgen-dependent and androgen-independent tumors as well as retarding the growth of already established tumors and prolong the survival of treated mice. Moreover, anti-103P3E8 mAbs demonstrate a dramatic inhibitory effect on the spread of local prostate tumor to distal sites, even in the presence of a large tumor burden. Thus, anti-103P3E8 mAbs are efficacious on major clinically relevant end points/PSA levels (tumor growth), prolongation of survival, and health.

### Example 18: Involvement of 103P3E8 in Protein-Protein Association

103P3E8 contains two EF hand motifs and a leucine zipper motif. Both of these motifs are correlated with mediation of protein-protein interactions. In addition, the Ras-like domain of 103P3E8 correlates with mediation of interactions with downstream effectors. The association of proteins into complexes is critical in several biological processes, including signal transduction, cell communication, ubiquitination, transcriptional regulation, etc.

It is determined whether 103P3E8 associates with specific proteins including cytoskeleton, filaments and signaling molecules using co-precipitation and Western blotting techniques (Hamilton B. J., et al. Biochem. Biophys. Res. Commun. 1999, 261:646). Immunoprecipitates from cells expressing 103P3E8 and cells lacking 103P3E8 are compared for specific protein-protein associations. 103P3E8 can specifically associate with GAP-like proteins, guanine nucleotide exchange factors (GNEFs) and protein kinases (Drugan JK. J Biol Chem. 2000, 275:35021). These interactions are studied by Western blotting using specific antibodies. Studies comparing 103P3E8 positive and 34P3D7 negative cells as well as studies comparing unstimulated/resting cells and cells treated with epithelial cell activators, such as cytokines, androgen and anti-integrin Ab reveal unique protein interactions. When 103P3E8 functions in protein-protein interactions, 103P3E8 is used as a target for diagnostic, prognostic, preventative and therapeutic purposes.

### Example 19: Involvement of 103P3E8 in Cellular Trafficking

Rab proteins are GTP binding proteins that regulate vesicular transport involved in endo- and exo-cytosis (Gonzalez L. Cell 1999, 96:755). The homology of 103P3E8 with Rab8 and the presence of EF hands suggest that 103P3E8 participates in the regulation of vesicular trafficking. In order to determine the contribution of 103P3E8 in vesicle movement, 103P3E8-expresing and 103P3E8-lacking cells are compared using bodipy-ceramide labeled bovine serum albumine (Huber L et al. Mol. Cell. Biol. 1995, 15:918). Briefly, cells are allowed to injest the labeled BSA and are placed intermittently at 4° C. and 18° C. to allow for trafficking to take place. Cells are examined under fluorescent microscopy at different time points for the presence of labeled BSA in specific vesicular compartments, including Golgi, endoplasmic reticulum, etc. In another embodiment, the effect of 103P3E8 on membrane transport is examined using biotin-avidin complexes. Cells either expressing or lacking 103P3E8 are transiently incubated with biotin. The cells are placed at 4° C. or transiently warmed to 37° C. for various periods of time. The cells are fractionated and examined by avidin affinity precipitation for the presence of biotin in specific cellular compartments. Using such assay sytems, proteins, antibodies and small molecules are identified that modify the effect of 103P3E8 on vesicular transport. When 103P3E8 functions in vesicular transport, 103P3E8 is used as a target for diagnostic, prognostic, preventative and therapeutic purposes.

Throughout this application, various publications and applications are referenced. The disclosures of these publications and applications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Tissues that Express 103P3E8 When Malignant

Prostate
Bladder
Kidney
Colon
Lung
Breast
Rectum
Stomach

TABLE II
AMINO ACID ABBREVIATIONS

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE III
AMINO ACID SUBSTITUTION MATRIX
Adapted from the GCG Software 9.0 BLOSUM62 amino acid
substitution matrix (block substitution matrix). The
higher the value, the more likely a substitution is found
in related, natural proteins.

| A | C | D | E | F | G | H | I | K | L | M | N | P | Q | R | S | T | V | W | Y | |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

TABLE IV (A)
HLA CLASS I SUPERMOTIFS

| SUPERMOTIF | POSITION 2 | C-TERMINUS |
|---|---|---|
| A2 | L,I,V,M,A,T,Q | L,.I,V,M,A,T |
| A3 | A,V,I,L,M,S,T | R,K |
| B7 | P | A,L,I,M,V,F,W,Y |
| B44 | D,E | F,W,Y,L,I,M,V,A |
| A1 | T,S,L,I,V,M | F,W,Y |
| A24 | F,W,Y,L,V,I,M,T | F,I,Y,W,L,M |
| B27 | R,H,K | A,L,I,V,M,Y,F,W |
| B58 | A,S,T | F,W,Y,L,I,V |
| B62 | L,V,M,P,I,Q | F,W,Y,M,I,V |

TABLE IV (B)
HLA CLASS II SUPERMOTIF

| 1 | 6 | 9 |
|---|---|---|
| W,F,Y,V,.I,L | A,V,I,L,P,C,S,T | A,V,I,L,C,S,T,M,Y |

TABLE V
HLA Peptide Scoring Results-103P3E8-A1-9-mers

| | | | Score (Estimate of Half Time |
|---|---|---|---|
| | | | of Disassociation of a |
| | Start | Subsequence Residue | Molecule Containing This |
| Rank | Position | Listing | Subsequence) |
| 1 | 50 | VAEPAGRAK | 180.000 |
| 2 | 705 | KADGVLLLY | 125.000 |
| 3 | 395 | ELDALKSDY | 25.000 |
| 4 | 372 | SMENQKVKK | 18.000 |
| 5 | 332 | ETEVGDLQV | 11.250 |
| 6 | 273 | STEMENLAI | 11.250 |
| 7 | 296 | ELEEEMDQR | 9.000 |
| 8 | 451 | ALENSYSKF | 9.000 |
| 9 | 393 | QSELDALKS | 6.750 |
| 10 | 96 | DGDGEELAR | 6.250 |
| 11 | 766 | FGEKLAMTY | 5.625 |
| 12 | 111 | ACDANRSGR | 5.000 |
| 13 | 509 | LCDPLQRTN | 5.000 |
| 14 | 620 | LVDDNAKSF | 5.000 |
| 15 | 31 | RREPLGHPR | 4.500 |
| 16 | 249 | LVEPRLIQP | 4.500 |
| 17 | 598 | VSEGSIVSS | 2.700 |
| 18 | 335 | VGDLQVTIK | 2.500 |
| 19 | 358 | KEDVAALKK | 2.500 |
| 20 | 275 | EMENLAIAV | 2.250 |
| 21 | 98 | DGEELARLR | 2.250 |
| 22 | 129 | CTELRVRPA | 2.250 |
| 23 | 236 | REEQVSTLY | 2.250 |
| 24 | 228 | GDEAKFIPR | 2.250 |
| 25 | 210 | SCGPASPGR | 2.000 |
| 26 | 413 | DLEIIRAYT | 1.800 |
| 27 | 357 | QKEDVAALK | 1.800 |
| 28 | 567 | ASDTDVPDI | 1.500 |
| 29 | 647 | SSFLMRLCK | 1.500 |
| 30 | 184 | VSEAGPETH | 1.350 |
| 31 | 542 | DSEVEYKHQ | 1.350 |
| 32 | 617 | QTDLVDDNA | 1.250 |
| 33 | 737 | ETVPIMLVG | 1.250 |
| 34 | 483 | GHSPQPLGY | 1.250 |
| 35 | 177 | PLDPAPAVS | 1.000 |
| 36 | 59 | LAGPPGGSR | 1.000 |
| 37 | 24 | GAGPNRRRR | 1.000 |
| 38 | 339 | QVTIKKLRK | 1.000 |
| 39 | 319 | KAEEALSDL | 0.900 |
| 40 | 382 | LLEAQTNIA | 0.900 |
| 41 | 544 | EVEYKHQRG | 0.900 |
| 42 | 789 | IVEAVLHLA | 0.900 |
| 43 | 778 | FCETSAKDG | 0.900 |
| 44 | 582 | GLEDVASVL | 0.900 |
| 45 | 347 | KLEEQSKRV | 0.900 |
| 46 | 138 | DAEAVFQRL | 0.900 |
| 47 | 730 | MIEDAAHET | 0.900 |
| 48 | 430 | QIEILQTAN | 0.900 |
| 49 | 476 | RSSPKFIGH | 0.750 |
| 50 | 468 | ISPGNTISR | 0.750 |

TABLE VI
HLA Peptide Scoring Results-103P3E8-A1-10-mers

| | | | Score (Estimate of Half Time |
|---|---|---|---|
| | | | of Disassociation of a |
| | Start | Subsequence Residue | Molecule Containing This |
| Rank | Position | Listing | Subsequence) |
| 1 | 249 | LVEPRLIQPY | 450.000 |
| 2 | 420 | YTEDRNSLER | 112.500 |
| 3 | 789 | IVEAVLHLAR | 45.000 |
| 4 | 382 | LLEAQTNIAF | 45.000 |
| 5 | 617 | QTDLVDDNAK | 25.000 |
| 6 | 430 | QIEILQTANR | 18.000 |
| 7 | 275 | EMENLAIAVK | 18.000 |
| 8 | 50 | VAEPAGRAKL | 18.000 |
| 9 | 542 | DSEVEYKHQR | 13.500 |
| 10 | 335 | VGDLQVTIKK | 12.500 |
| 11 | 409 | NTERDLEIIR | 11.250 |
| 12 | 31 | RREPLGHPRR | 9.000 |
| 13 | 319 | KAEEALSDLR | 9.000 |
| 14 | 544 | EVEYKHQRGF | 9.000 |
| 15 | 567 | ASDTDVPDIR | 7.500 |
| 16 | 294 | LSELEEEMDQ | 6.750 |
| 17 | 227 | LGDEAKFIPR | 6.250 |
| 18 | 403 | YADQSLNTER | 5.000 |
| 19 | 519 | EVDSLPESCF | 5.000 |
| 20 | 467 | NISPGNTISR | 5.000 |
| 21 | 509 | LCDPLQRTNC | 5.000 |
| 22 | 273 | STEMENLAIA | 4.500 |
| 23 | 755 | ATEGQKCVPG | 4.500 |
| 24 | 357 | QKEDVAALKK | 4.500 |
| 25 | 254 | LIQPYEHVIK | 4.000 |
| 26 | 147 | DADRDGAITF | 2.500 |
| 27 | 136 | PADAEAVFQR | 2.500 |
| 28 | 320 | AEEALSDLRR | 2.250 |
| 29 | 538 | PNEYDSEVEY | 2.250 |
| 30 | 332 | ETEVGDLQVT | 2.250 |
| 31 | 760 | KCVPGHFGEK | 2.000 |
| 32 | 517 | NCEVDSLPES | 1.800 |
| 33 | 696 | RSIAKSYFRK | 1.500 |
| 34 | 324 | LSDLRRQYET | 1.500 |
| 35 | 646 | KSSFLMRLCK | 1.500 |
| 36 | 598 | VSEGSIVSSS | 1.350 |
| 37 | 691 | GQERFRSIAK | 1.350 |
| 38 | 411 | ERDLEIIRAY | 1.250 |
| 39 | 572 | VPDIRDEETF | 1.250 |
| 40 | 655 | KNEFRENISA | 1.125 |
| 41 | 98 | DGEELARLRS | 1.125 |
| 42 | 727 | WVDMIEDAAH | 1.000 |
| 43 | 676 | IVDGERTVLQ | 1.000 |
| 44 | 747 | KADIRDTAAT | 1.000 |
| 45 | 347 | KLEEQSKRVS | 0.900 |
| 46 | 716 | TCEKSFLNIR | 0.900 |
| 47 | 730 | MIEDAAHETV | 0.900 |
| 48 | 296 | ELEEEMDQRI | 0.900 |
| 49 | 778 | FCETSAKDGS | 0.900 |
| 50 | 797 | AREVKKRTDK | 0.900 |

TABLE VII
HLA Peptide Scoring Results-103P3E8-A2-9-mers

| | | | Score (Estimate of Half Time |
|---|---|---|---|
| | | | of Disassociation of a |
| | Start | Subsequence Residue | Molecule Containing This |
| Rank | Position | Listing | Subsequence) |
| 1 | 242 | TLYQNINLV | 511.903 |
| 2 | 226 | RLGDEAKFI | 235.260 |
| 3 | 440 | KLHDSNDGL | 150.210 |
| 4 | 391 | FLQSELDAL | 110.747 |
| 5 | 581 | FGLEDVASV | 72.870 |
| 6 | 508 | ALCDPLQRT | 70.272 |
| 7 | 347 | KLEEQSKRV | 63.877 |
| 8 | 253 | RLIQPYEHV | 51.121 |
| 9 | 636 | IVLAGDAAV | 38.280 |
| 10 | 613 | ALSPQTDLV | 34.080 |
| 11 | 709 | VLLLYDVTC | 31.249 |
| 12 | 788 | NIVEAVLHL | 27.699 |
| 13 | 771 | AMTYGALFC | 19.734 |
| 14 | 742 | MLVGNKADI | 17.736 |
| 15 | 769 | KLAMTYGAL | 14.580 |
| 16 | 481 | FIGHSPQPL | 13.512 |
| 17 | 675 | LIVDGERTV | 13.331 |
| 18 | 381 | DLLEAQTNI | 11.870 |
| 19 | 278 | NLAIAVKRA | 11.426 |
| 20 | 293 | QLSELEEEM | 9.981 |
| 21 | 433 | ILQTANRKL | 7.263 |
| 22 | 499 | YVDEDCDSL | 6.910 |
| 23 | 176 | GPLDPAPAV | 6.887 |
| 24 | 370 | DLSMENQKV | 5.216 |
| 25 | 258 | YEHVIKNFI | 5.132 |
| 26 | 429 | RQIEILQTA | 4.750 |
| 27 | 792 | AVLHLAREV | 4.503 |
| 28 | 271 | LQSTEMENL | 4.150 |
| 29 | 754 | AATEGQKCV | 3.961 |
| 30 | 337 | DLQVTIKKL | 3.685 |
| 31 | 674 | TLIVDGERT | 3.651 |
| 32 | 126 | RALCTELRV | 3.574 |
| 33 | 246 | NINLVEPRL | 2.937 |
| 34 | 388 | NIAFLQSEL | 2.937 |
| 35 | 183 | AVSEAGPET | 2.673 |
| 36 | 643 | AVGKSSFLM | 2.521 |
| 37 | 289 | KAAMQLSEL | 2.388 |
| 38 | 582 | GLEDVASVL | 2.298 |
| 39 | 119 | RLEREEFRA | 1.844 |
| 40 | 263 | KNFIREIRL | 1.806 |
| 41 | 681 | RTVLQLWDT | 1.785 |
| 42 | 522 | SLPESCFDS | 1.772 |
| 43 | 363 | ALKKQIYDL | 1.720 |
| 44 | 667 | GVDFQMKTL | 1.720 |
| 45 | 551 | RGFQRSHGV | 1.680 |
| 46 | 642 | AAVGKSSFL | 1.632 |
| 47 | 603 | IVSSSRKPI | 1.552 |
| 48 | 512 | PLQRTNCEV | 1.530 |
| 49 | 664 | ATLGVDFQM | 1.481 |
| 50 | 274 | TEMENLAIA | 1.382 |

TABLE VIII
HLA Peptide Scoring Results-A2-103P3E8-10-mers

| | | | Score (Estimate of Half Time |
|---|---|---|---|
| | | | of Disassociation of a |
| | Start | Subsequence Residue | Molecule Containing This |
| Rank | Position | Listing | Subsequence) |
| 1 | 741 | IMLVGNKADI | 47.394 |
| 2 | 721 | FLNIREWVDM | 38.850 |
| 3 | 635 | KIVLAGDAAV | 33.472 |
| 4 | 665 | TLGVDFQMKT | 28.318 |
| 5 | 331 | YETEVGDLQV | 25.506 |
| 6 | 270 | RLQSTEMENL | 24.075 |
| 7 | 274 | TEMENLAIAV | 20.516 |
| 8 | 719 | KSFLNIREWV | 15.845 |
| 9 | 674 | TLIVDGERTV | 13.910 |
| 10 | 407 | SLNTERDLEI | 10.433 |
| 11 | 218 | RAWQDFQARL | 9.358 |
| 12 | 383 | LEAQTNIAFL | 8.933 |
| 13 | 563 | FGGDASDTDV | 8.563 |
| 14 | 329 | RQYETEVGDL | 8.497 |
| 15 | 6 | LLGGAWSPGA | 8.446 |
| 16 | 675 | LIVDGERTVL | 8.394 |
| 17 | 241 | STLYQNINLV | 8.221 |
| 18 | 473 | TISRSSPKFI | 7.890 |
| 19 | 788 | NIVEAVLHLA | 6.442 |
| 20 | 589 | VLDWKPQGSV | 6.148 |
| 21 | 526 | SCFDSGLSTL | 4.866 |
| 22 | 233 | FIPREEQVST | 4.713 |
| 23 | 253 | RLIQPYEHVI | 4.277 |
| 24 | 581 | FGLEDVASVL | 3.990 |
| 25 | 761 | CVPGHFGEKL | 3.480 |
| 26 | 265 | FIREIRLQST | 3.378 |
| 27 | 499 | YVDEDCDSLA | 3.279 |
| 28 | 362 | AALKKQIYDL | 2.525 |
| 29 | 700 | KSYFRKADGV | 2.492 |
| 30 | 770 | LAMTYGALFC | 2.387 |
| 31 | 381 | DLLEAQTNIA | 2.317 |
| 32 | 666 | LGVDFQMKTL | 2.236 |
| 33 | 730 | MIEDAAHETV | 2.090 |
| 34 | 175 | WGPLDPAPAV | 2.088 |
| 35 | 101 | ELARLRSVFA | 2.049 |
| 36 | 715 | VTCEKSFLNI | 1.919 |
| 37 | 612 | SALSPQTDLV | 1.751 |
| 38 | 729 | DMIEDAAHET | 1.655 |
| 39 | 369 | YDLSMENQKV | 1.644 |
| 40 | 708 | GVLLLYDVTC | 1.608 |
| 41 | 292 | MQLSELEEEM | 1.552 |
| 42 | 507 | LALCDPLQRT | 1.497 |
| 43 | 346 | RKLEEQSKRV | 1.465 |
| 44 | 602 | SIVSSSRKPI | 1.435 |
| 45 | 93 | MEADGDGEEL | 1.419 |
| 46 | 659 | RENISATLGV | 1.352 |
| 47 | 628 | FSSQKAYKIV | 1.248 |
| 48 | 649 | FLMRLCKNEF | 1.268 |
| 49 | 656 | NEFRENISAT | 1.233 |
| 50 | 285 | RAQDKAAMQL | 1.216 |

TABLE IX
HLA Peptide Scoring Results-103P3E8-A3-9-mers

| | | | Score (Estimate of Half Time |
|---|---|---|---|
| | | | of Disassociation of a |
| | Start | Subsequence Residue | Molecule Containing This |
| Rank | Position | Listing | Subsequence) |
| 1 | 711 | LLYDVTCEK | 150.000 |
| 2 | 776 | ALFCETSAK | 100.000 |
| 3 | 665 | TLGVDFQMK | 60.000 |
| 4 | 814 | NLTGTNSKK | 30.000 |
| 5 | 372 | SMENQKVKK | 20.000 |
| 6 | 793 | VLHLAREVK | 20.000 |
| 7 | 795 | HLAREVKKR | 6.000 |
| 8 | 697 | SIAKSYFRK | 6.000 |
| 9 | 22 | LLGAGPNRR | 4.000 |
| 10 | 339 | QVTIKKLRK | 4.000 |
| 11 | 296 | ELEEEMDQR | 2.700 |
| 12 | 761 | CVPGHFGEK | 2.700 |
| 13 | 582 | GLEDVASVL | 2.700 |
| 14 | 363 | ALKKQIYDL | 2.700 |
| 15 | 21 | DLLGAGPNR | 2.700 |
| 16 | 451 | ALENSYSKF | 2.000 |
| 17 | 650 | LMRLCKNEF | 2.000 |
| 18 | 440 | KLHDSNDGL | 1.800 |
| 19 | 311 | KTRKDEKRK | 1.500 |
| 20 | 472 | NTISRSSPK | 1.500 |
| 21 | 242 | TLYQNINLV | 1.500 |
| 22 | 585 | DVASVLDWK | 1.350 |
| 23 | 450 | SALENSYSK | 1.350 |
| 24 | 742 | MLVGNKADI | 1.350 |
| 25 | 416 | IIRAYTEDR | 1.200 |
| 26 | 153 | AITFQEFAR | 1.200 |
| 27 | 163 | FLGSLRGGR | 1.200 |
| 28 | 395 | ELDALKSDY | 1.200 |
| 29 | 647 | SSFLMRLCK | 1.000 |
| 30 | 426 | SLERQIEIL | 0.900 |
| 31 | 104 | RLRSVFAAC | 0.900 |
| 32 | 391 | FLQSELDAL | 0.900 |
| 33 | 709 | VLLLYDVTC | 0.900 |
| 34 | 392 | LQSELDALK | 0.900 |
| 35 | 432 | EILQTANRK | 0.900 |
| 36 | 788 | NIVEAVLHL | 0.810 |
| 37 | 723 | NIREWVDMI | 0.810 |
| 38 | 705 | KADGVLLLY | 0.810 |
| 39 | 119 | RLEREEFRA | 0.600 |
| 40 | 101 | ELARLRSVF | 0.600 |

TABLE X
HLA Peptide Scoring Results-103P3E8-A3-10-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 344 | KLRKLEEQSK | 60.000 |
| 2 | 710 | LLLYDVTCEK | 45.000 |
| 3 | 637 | VLAGDAAVGK | 30.000 |
| 4 | 685 | QLWDTAGQER | 20.000 |
| 5 | 391 | FLQSELDALK | 20.000 |
| 6 | 793 | VLHLAREVKK | 20.000 |
| 7 | 769 | KLAMTYGALF | 18.000 |
| 8 | 447 | GLRSALENSY | 12.000 |
| 9 | 742 | MLVGNKADIR | 9.000 |
| 10 | 275 | EMENLAIAVK | 9.000 |
| 11 | 58 | KLAGPPGGSR | 8.100 |
| 12 | 506 | SLALCDPLQR | 8.000 |
| 13 | 664 | ATLGVDFQMK | 6.750 |
| 14 | 650 | LMRLCKNEFR | 4.000 |
| 15 | 382 | LLEAQTNIAF | 4.000 |
| 16 | 691 | GQERFRSIAK | 3.600 |
| 17 | 440 | KLHDSNDGLR | 3.600 |
| 18 | 649 | FLMRLCKNEF | 3.000 |
| 19 | 792 | AVLHLAREVK | 3.000 |
| 20 | 254 | LIQPYEHVIK | 3.000 |
| 21 | 370 | DLSMENQKVK | 3.000 |
| 22 | 21 | DLLGAGPNRR | 2.700 |
| 23 | 253 | RLIQPYEHVI | 2.700 |
| 24 | 643 | AVGKSSFLMR | 2.400 |
| 25 | 141 | AVFQRLDADR | 2.000 |
| 26 | 280 | AJAVKRAQDK | 2.000 |
| 27 | 107 | SVFAACDANR | 2.000 |
| 28 | 738 | TVPIMLVGNK | 1.800 |
| 29 | 338 | LQVTIKKLRK | 1.800 |
| 30 | 334 | EVGDLQVTIK | 1.800 |
| 31 | 626 | KSFSSQKAYK | 1.500 |
| 32 | 356 | SQKEDVAALK | 1.350 |
| 33 | 760 | KCVPGHFGEK | 1.215 |
| 34 | 163 | FLGSLRGGRR | 1.200 |
| 35 | 467 | NISPGNTISR | 1.200 |
| 36 | 407 | SLNTERDLEI | 1.200 |
| 37 | 617 | QTDLVDDNAK | 1.000 |
| 38 | 812 | ITNLTGTNSK | 1.000 |
| 39 | 741 | IMLVGNKADI | 0.900 |
| 40 | 6 | LLGGAWSPGA | 0.900 |
| 41 | 249 | LVEPRLIQPY | 0.900 |
| 42 | 270 | RLQSTEMENL | 0.900 |
| 43 | 775 | GALFCETSAK | 0.900 |
| 44 | 337 | DLQVTLKKLR | 0.900 |
| 45 | 539 | NEYDSEVEYK | 0.900 |
| 46 | 789 | IVEAVLHLAR | 0.800 |
| 47 | 305 | IQAAEHKTRK | 0.600 |
| 48 | 646 | KSSFLMRLCK | 0.600 |
| 49 | 223 | FQARLGDEAK | 0.600 |
| 50 | 22 | LLGAGPNRRR | 0.600 |

TABLE XI
HLA Peptide Scoring Results-103P3E8-A11-9-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 339 | QVTIKKLRK | 4.000 |
| 2 | 761 | CVPGHFGEK | 2.000 |
| 3 | 472 | NTISRSSPK | 1.500 |
| 4 | 311 | KTRKDEKRK | 1.500 |
| 5 | 697 | SIAKSYFRK | 1.200 |
| 6 | 673 | KTLIVDGER | 0.900 |
| 7 | 776 | ALFCETSAK | 0.800 |
| 8 | 711 | LLYDVTCEK | 0.800 |
| 9 | 585 | DVASVLDWK | 0.600 |
| 10 | 450 | SALENSYSK | 0.600 |
| 11 | 255 | IQPYEHVIK | 0.600 |
| 12 | 392 | LQSELDALK | 0.600 |
| 13 | 793 | VLHLAREVK | 0.400 |
| 14 | 665 | TLGVDFQMK | 0.400 |
| 15 | 743 | LVGNKADIR | 0.400 |
| 16 | 814 | NLTGTNSKK | 0.400 |
| 17 | 372 | SMENQKVKK | 0.400 |
| 18 | 358 | KEDVAALKK | 0.360 |
| 19 | 752 | DTAATEGQK | 0.300 |
| 20 | 281 | IAVKRAQDK | 0.300 |
| 21 | 739 | VPIMLVGNK | 0.300 |
| 22 | 798 | REVKKRTDK | 0.270 |
| 23 | 153 | AITFQEFAR | 0.240 |
| 24 | 218 | RAWQDFQAR | 0.240 |
| 25 | 624 | NAKSFSSQK | 0.200 |
| 26 | 306 | QAAEHKTRK | 0.200 |
| 27 | 638 | LAGDAAVGK | 0.200 |
| 28 | 627 | SFSSQKAYK | 0.200 |
| 29 | 224 | QARLGDEAK | 0.200 |
| 30 | 336 | GDLQVTIKK | 0.180 |
| 31 | 432 | EILQTANRK | 0.180 |
| 32 | 350 | EQSKRVSQK | 0.180 |
| 33 | 540 | EYDSEVEYK | 0.120 |
| 34 | 692 | QERFRSIAK | 0.120 |
| 35 | 377 | KVKKDLLEA | 0.120 |
| 36 | 507 | LALCDPLQR | 0.120 |
| 37 | 601 | GSIVSSSRK | 0.090 |
| 38 | 338 | LQVTIKKLR | 0.090 |
| 39 | 647 | SSFLMRLGK | 0.080 |
| 40 | 163 | FLGSLRGGR | 0.080 |
| 41 | 22 | LLGAGPNRR | 0.080 |
| 42 | 485 | SPQPLGYDR | 0.080 |
| 43 | 416 | IIRAYTEDR | 0.080 |
| 44 | 813 | TNLTGTNSK | 0.060 |
| 45 | 643 | AVGKSSFLM | 0.060 |
| 46 | 354 | RVSQKEDVA | 0.060 |
| 47 | 276 | MENLATAVK | 0.060 |
| 48 | 133 | RVRPADAEA | 0.060 |
| 49 | 305 | IQAAEHKTR | 0.060 |
| 50 | 664 | ATLGVDFQM | 0.045 |

TABLE XII
HLA Peptide Scoring Results-103P3E8-A11-10-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 691 | GQERFRSIAK | 3.600 |
| 2 | 792 | AVLHLAREVK | 3.000 |
| 3 | 738 | TVPIMLVGNK | 2.000 |
| 4 | 338 | LQVTIKKLRK | 1.800 |
| 5 | 664 | ATLGVDFQMK | 1.500 |
| 6 | 344 | KLRKLEEQSK | 1.200 |
| 7 | 812 | ITNLTGTNSK | 1.000 |
| 8 | 617 | QTDLVDDNAK | 1.000 |
| 9 | 760 | KCVPGHFGEK | 0.900 |
| 10 | 775 | GALFCETSAK | 0.900 |
| 11 | 643 | AVGKSSFLMR | 0.800 |
| 12 | 107 | SVFAACDANR | 0.800 |
| 13 | 789 | IVEAVLHLAR | 0.800 |
| 14 | 141 | AVFQRLDADR | 0.800 |
| 15 | 356 | SQKEDVAALK | 0.600 |
| 16 | 710 | LLLYDVTCEK | 0.600 |
| 17 | 305 | IQAAEHKTRK | 0.600 |
| 18 | 223 | FQARLGDEAK | 0.600 |
| 19 | 334 | EVGDLQVTIK | 0.600 |
| 20 | 152 | GAITFQEFAR | 0.540 |
| 21 | 368 | IYDLSMENQK | 0.400 |
| 22 | 254 | LIQPYEHVIK | 0.400 |
| 23 | 793 | VLHLAREVKK | 0.400 |
| 24 | 280 | AIAVKRAQDK | 0.400 |
| 25 | 409 | NTERDLEIIR | 0.400 |
| 26 | 420 | YTEDRNSLER | 0.400 |
| 27 | 391 | FLQSELDALK | 0.400 |
| 28 | 637 | VLAGDAAVGK | 0.400 |
| 29 | 696 | RSIAKSYFRK | 0.270 |
| 30 | 158 | KLAGPPGGSR | 0.240 |
| 31 | 440 | KLHDSNDGLR | 0.240 |
| 32 | 9 | GAWSPGAPHR | 0.240 |
| 33 | 61 | GPPGGSRWPR | 0.240 |
| 34 | 49 | QVAEPAGRAK | 0.200 |
| 35 | 162 | GFLGSLRGGR | 0.180 |
| 36 | 302 | DQRIQAAEHK | 0.180 |
| 37 | 467 | NISPGNTISR | 0.160 |
| 38 | 506 | SLALCDPLQR | 0.160 |
| 39 | 685 | QLWDTAGQER | 0.160 |
| 40 | 646 | KSSFLMRLCK | 0.120 |
| 41 | 449 | RSALENSYSK | 0.120 |
| 42 | 471 | GNTISRSSPK | 0.120 |
| 43 | 626 | KSFSSQKAYK | 0.120 |
| 44 | 539 | NEYDSEVEYK | 0.120 |
| 45 | 742 | MLVGNKADIR | 0.120 |
| 46 | 244 | YQNINLVEPR | 0.120 |
| 47 | 319 | KAEEALSDLR | 0.120 |
| 48 | 70 | RPSREGPAPR | 0.120 |
| 49 | 304 | RIQAAEHKTR | 0.120 |
| 50 | 275 | EMENLAIAVK | 0.120 |

TABLE XIII
HLA Peptide Scoring Results-103P3E8-A24-9-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 330 | QYETEVGDL | 300.000 |
| 2 | 419 | AYTEDRNSL | 288.000 |
| 3 | 455 | SYSKFNRSL | 200.000 |
| 4 | 124 | EFRALCTEL | 26.400 |
| 5 | 257 | PYEHVIKNF | 21.000 |
| 6 | 702 | YFRKADGVL | 20.000 |
| 7 | 527 | CFDSGLSTL | 20.000 |
| 8 | 155 | TFQEFARGF | 18.000 |
| 9 | 319 | KAEEALSDL | 14.400 |
| 10 | 138 | DAEAVFQRL | 12.096 |
| 11 | 515 | RTNCEVDSL | 12.000 |
| 12 | 582 | GLEDVASVL | 10.080 |
| 13 | 400 | KSDYADQSL | 9.600 |
| 14 | 440 | KLHDSNDGL | 9.600 |
| 15 | 498 | SYVDEDCDS | 9.000 |
| 16 | 289 | KAAMQLSEL | 8.800 |
| 17 | 246 | NINLVEPRL | 8.400 |
| 18 | 374 | ENQKVKKDL | 8.400 |
| 19 | 786 | GSNIVEAVL | 8.400 |
| 20 | 263 | KNFIREIRL | 8.000 |
| 21 | 769 | KLAMTYGAL | 8.000 |
| 22 | 773 | TYGALFCET | 7.920 |
| 23 | 505 | DSLALCDPL | 7.200 |
| 24 | 219 | AWQDFQARL | 7.200 |
| 25 | 788 | NIVEAVLHL | 7.200 |
| 26 | 337 | DLQVTIKKL | 6.600 |
| 27 | 433 | ILQTANRKL | 6.600 |
| 28 | 340 | VTIKKLRKL | 6.600 |
| 29 | 14 | GAPHRSRDL | 6.000 |
| 30 | 402 | DYADQSLNT | 6.000 |
| 31 | 391 | FLQSELDAL | 6.000 |
| 32 | 612 | SALSPQTDL | 6.000 |
| 33 | 156 | FQEFARGFL | 6.000 |
| 34 | 642 | AAVGKSSFL | 6.000 |
| 35 | 678 | DGERTVLQL | 6.000 |
| 36 | 406 | QSLNTERDL | 6.000 |
| 37 | 426 | SLERQIEIL | 6.000 |
| 38 | 384 | EAQTNIAFL | 6.000 |
| 39 | 241 | STLYQNTNL | 6.000 |
| 40 | 555 | RSHGVQESF | 5.600 |
| 41 | 712 | LYDVTCEKS | 5.500 |
| 42 | 388 | NJAFLQSEL | 5.280 |
| 43 | 490 | GYDRSSRSS | 5.000 |
| 44 | 701 | SYFRKADGV | 5.000 |
| 45 | 444 | SNDGLRSAL | 4.800 |
| 46 | 356 | SQKEDVAAL | 4.800 |
| 47 | 135 | RPADAEAVF | 4.800 |
| 48 | 481 | FIGHSPQPL | 4.800 |
| 49 | 499 | YVDEDCDSL | 4.800 |
| 50 | 94 | EADGDGEEL | 4.400 |

TABLE XIV
HLA Peptide Scoring Results-103P3E8-A24-10-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 498 | SYVDEDCDSL | 360.000 |
| 2 | 701 | SYFRKADGVL | 200.000 |
| 3 | 712 | LYDVTCEKSF | 100.000 |
| 4 | 480 | KFIGHSPQPL | 72.000 |
| 5 | 155 | TFQEFARGFL | 36.000 |
| 6 | 657 | EFRENISATL | 33.600 |
| 7 | 390 | AFLQSELDAL | 30.000 |
| 8 | 158 | EFARGFLGSL | 20.000 |
| 9 | 694 | RFRSIAKSYF | 20.000 |
| 10 | 702 | YFRKADGVLL | 20.000 |
| 11 | 119 | RLEREEFRAL | 14.400 |
| 12 | 285 | RAQDKAAMQL | 14.400 |
| 13 | 257 | PYEHVIKNFI | 12.600 |
| 14 | 270 | RLQSTEMENL | 12.000 |
| 15 | 581 | FGLEDVASVL | 10.080 |
| 16 | 418 | RAYTEDRNSL | 9.600 |
| 17 | 329 | RQYETEVGDL | 9.600 |
| 18 | 443 | DSNDGLRSAL | 8.640 |
| 19 | 245 | QNINLVEPRL | 8.400 |
| 20 | 218 | RAWQDFQARL | 8.000 |
| 21 | 387 | TNIAFLQSEL | 7.920 |
| 22 | 50 | VAEPAGRAKL | 7.920 |
| 23 | 425 | NSLERQIEIL | 7.200 |
| 24 | 675 | LIVDGERTVL | 7.200 |
| 25 | 666 | LGVDFQMKTL | 7.200 |
| 26 | 734 | AAHETVPIML | 6.720 |
| 27 | 432 | EILQTANRKL | 6.600 |
| 28 | 761 | CVPGHFGEKL | 6.600 |
| 29 | 26 | GPNRRRREPL | 6.000 |
| 30 | 374 | ENQKVKKDLL | 6.000 |
| 31 | 362 | AALKKQIYDL | 6.000 |
| 32 | 787 | SNIVEAVLHL | 6.000 |
| 33 | 14 | GAPHRSRDLL | 6.000 |
| 34 | 629 | SSQKAYKIVL | 6.000 |
| 35 | 523 | LPESCFDSGL | 6.000 |
| 36 | 355 | VSQKEDVAAL | 6.000 |
| 37 | 633 | AYKIVLAGDA | 6.000 |
| 38 | 785 | DGSNIVEAVL | 5.600 |
| 39 | 627 | SFSSQKAYKI | 5.500 |
| 40 | 490 | GYDRSSRSSY | 5.000 |
| 41 | 773 | TYGALFCETS | 5.000 |
| 42 | 526 | SCFDSGLSTL | 4.800 |
| 43 | 574 | DIRDEETFGL | 4.800 |
| 44 | 234 | IPREEQVSTL | 4.800 |
| 45 | 198 | EGDEDAAAAL | 4.800 |
| 46 | 339 | QVTIKKLRKL | 4.400 |
| 47 | 116 | RSGRLEREEF | 4.400 |
| 48 | 757 | EGQKCVPGHF | 4.200 |
| 49 | 769 | KLAMTYGALF | 4.000 |
| 50 | 605 | SSSRKPISAL | 4.000 |

TABLE XV
HLA Peptide Scoring Results-103P3E8-B7-9-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 15 | APHRSRDLL | 360.000 |
| 2 | 159 | FARGFLGSL | 120.000 |
| 3 | 762 | VPGHFGEKL | 80.000 |
| 4 | 606 | SSRKPISAL | 60.000 |
| 5 | 42 | SPRPTFPQV | 40.000 |
| 6 | 642 | AAVGKSSFL | 36.000 |
| 7 | 714 | DVTCEKSFL | 20.000 |
| 8 | 234 | IPREEQVST | 20.000 |
| 9 | 612 | SALSPQTDL | 18.000 |
| 10 | 643 | AVGKSSFLM | 15.000 |
| 11 | 289 | KAAMQLSEL | 12.000 |
| 12 | 14 | GAPHRSRDL | 12.000 |
| 13 | 384 | EAQTNIAFL | 12.000 |
| 14 | 77 | APRGAPEPS | 12.000 |
| 15 | 363 | ALKKQIYDL | 12.000 |
| 16 | 268 | EIRLQSTEM | 10.000 |
| 17 | 734 | AAHETVPIM | 9.000 |
| 18 | 676 | IVDGERTVL | 9.000 |
| 19 | 176 | GPLDPAPAV | 6.000 |
| 20 | 667 | GVDFQMKTL | 6.000 |
| 21 | 499 | YVDEDCDSL | 6.000 |
| 22 | 27 | PNRRRREPL | 6.000 |
| 23 | 133 | RVRPADAEA | 5.000 |
| 24 | 241 | STLYQNINL | 4.000 |
| 25 | 433 | ILQTANRKL | 4.000 |
| 26 | 391 | FLQSELDAL | 4.000 |
| 27 | 263 | KNFIREIRL | 4.000 |
| 28 | 630 | SQKAYKIVL | 4.000 |
| 29 | 440 | KLHDSNDGL | 4.000 |
| 30 | 170 | GRRRDWGPL | 4.000 |
| 31 | 505 | DSLALCDPL | 4.000 |
| 32 | 481 | FIGHSPQPL | 4.000 |
| 33 | 788 | NJVEAVLHL | 4.000 |
| 34 | 537 | DPNEYDSEV | 4.000 |
| 35 | 124 | EFRALCTEL | 4.000 |
| 36 | 375 | NQKVKKDLL | 4.000 |
| 37 | 356 | SQKEDVAAL | 4.000 |
| 38 | 246 | NTNLVEPRL | 4.000 |
| 39 | 769 | KLAMTYGAL | 4.000 |
| 40 | 474 | ISRSSPKFI | 4.000 |
| 41 | 786 | GSNIVEAVL | 4.000 |
| 42 | 340 | VTIKKLRKL | 4.000 |
| 43 | 723 | NIREWYDMI | 4.000 |
| 44 | 316 | EKRKAEEAL | 4.000 |
| 45 | 388 | NIAFLQSEL | 4.000 |
| 46 | 271 | LQSTEMENL | 4.000 |
| 47 | 120 | LEREEFRAL | 4.000 |
| 48 | 374 | ENQKVKKDL | 4.000 |
| 49 | 406 | QSLNTERDL | 4.000 |
| 50 | 515 | RTNCEVDSL | 4.000 |

TABLE XVI
HLA Peptide Scoring Results-103P3E8-B7-10-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 234 | IPREEQVSTL | 800.000 |
| 2 | 26 | GPNRRRREPL | 120.000 |
| 3 | 574 | DIRDEETFGL | 40.000 |
| 4 | 169 | GGRRRDWGPL | 40.000 |
| 5 | 362 | AALKKQIYDL | 36.000 |
| 6 | 734 | AAHETVPIML | 36.000 |
| 7 | 523 | LPESCFDSGL | 24.000 |
| 8 | 761 | GVPGHFGEKL | 20.000 |
| 9 | 68 | WPRPSREGPA | 20.000 |
| 10 | 42 | SPRPTFPQVA | 20.000 |
| 11 | 339 | QVTIKKLRKL | 20.000 |
| 12 | 37 | HPRRSSPRPT | 20.000 |
| 13 | 418 | RAYTEDRNSL | 18.000 |
| 14 | 14 | GAPHRSRDLL | 18.000 |
| 15 | 285 | RAQDKAAMQL | 12.000 |
| 16 | 218 | RAWQDFQARL | 12.000 |
| 17 | 641 | DAAVGKSSFL | 12.000 |
| 18 | 133 | RVRPADAEAV | 10.000 |
| 19 | 77 | APRGAPEPSR | 9.000 |
| 20 | 642 | AAVGKSSFLM | 9.000 |
| 21 | 605 | SSSRKPISAL | 6.000 |
| 22 | 611 | ISALSPQTDL | 6.000 |
| 23 | 443 | DSNDGLRSAL | 6.000 |
| 24 | 675 | LIVDGERTVL | 6.000 |
| 25 | 50 | VAEPAGRAKL | 5.400 |
| 26 | 454 | NSYSKFNRSL | 4.000 |
| 27 | 329 | RQYETEVGDL | 4.000 |
| 28 | 581 | FGLEDVASVL | 4.000 |
| 29 | 459 | FNRSLHINNI | 4.000 |
| 30 | 240 | VSTLYQNINL | 4.000 |
| 31 | 270 | RLQSTEMENL | 4.000 |
| 32 | 657 | EFRENISATL | 4.000 |
| 33 | 785 | DGSNIVEAVL | 4.000 |
| 34 | 702 | YFRIKADGVL | 4.000 |
| 35 | 526 | SCFDSGLSTL | 4.000 |
| 36 | 355 | VSQKEDVAAL | 4.000 |
| 37 | 387 | TNIAFLQSEL | 4.000 |
| 38 | 787 | SNIVEAVLHL | 4.000 |
| 39 | 245 | QNINLVEPRL | 4.000 |
| 40 | 644 | VGKSSFLMIR | 4.000 |
| 41 | 666 | LGVDFQMKTL | 4.000 |
| 42 | 425 | NSLERQIEIL | 4.000 |
| 43 | 511 | DPLQRTNCEV | 4.000 |
| 44 | 432 | EILQTANRKL | 4.000 |
| 45 | 374 | ENQKVKKDLL | 4.000 |
| 46 | 629 | SSQKAYKIVL | 4.000 |
| 47 | 405 | DQSLNTERDL | 4.000 |
| 48 | 111 | ACDANRSGRL | 3.600 |
| 49 | 663 | SATLGVDFQM | 3.000 |
| 50 | 102 | LARLRSVFAA | 3.000 |

TABLE XVII
HLA Peptide Scoring Results-103P3E8-B35-9-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 135 | RPADAEAVF | 80.000 |
| 2 | 215 | SPGRAWQDF | 20.000 |
| 3 | 15 | APHRSRDLL | 20.000 |
| 4 | 626 | KSFSSQKAY | 20.000 |
| 5 | 762 | VPGHFGEKL | 20.000 |
| 6 | 234 | IPREEQVST | 18.000 |
| 7 | 606 | SSRKPISAL | 15.000 |
| 8 | 42 | SPRPTFPQV | 12.000 |
| 9 | 734 | AAHETVPIM | 12.000 |
| 10 | 212 | GPASPGRAW | 10.000 |
| 11 | 555 | RSHGVQESF | 10.000 |
| 12 | 159 | FARGFLGSL | 9.000 |
| 13 | 356 | SQKEDVAAL | 9.000 |
| 14 | 537 | DPNEYDSEV | 8.000 |
| 15 | 176 | GPLDPAPAV | 8.000 |
| 16 | 289 | KAAMQLSEL | 6.000 |
| 17 | 474 | ISRSSPKFI | 6.000 |
| 18 | 361 | VAALKKQIY | 6.000 |
| 19 | 268 | EIRLQSTEM | 6.000 |
| 20 | 77 | APRGAPEPS | 6.000 |
| 21 | 406 | QSLNTERDL | 5.000 |
| 22 | 719 | KSFLNIREW | 5.000 |
| 23 | 662 | ISATLGVDF | 5.000 |
| 24 | 786 | GSNIVEAVL | 5.000 |
| 25 | 505 | DSLALCDPL | 5.000 |
| 26 | 117 | SGRLEREEF | 4.500 |
| 27 | 293 | QLSELEEEM | 4.000 |
| 28 | 440 | KLHDSNDGL | 4.000 |
| 29 | 256 | QPYEHVIKN | 4.000 |
| 30 | 323 | ALSDLRRQY | 4.000 |
| 31 | 425 | NSLERQIEI | 4.000 |
| 32 | 782 | SAKDGSNIV | 3.600 |
| 33 | 319 | KAEEALSDL | 3.600 |
| 34 | 705 | KADGVLLLY | 3.600 |
| 35 | 363 | ALKKQIYDL | 3.000 |
| 36 | 642 | AAVGKSSFL | 3.000 |
| 37 | 375 | NQKVKKDLL | 3.000 |
| 38 | 14 | GAPHRSRDL | 3.000 |
| 39 | 630 | SQKAYKIVL | 3.000 |
| 40 | 515 | RTNCEVDSL | 3.000 |
| 41 | 612 | SALSPQTDL | 3.000 |
| 42 | 781 | TSAKDGSNI | 3.000 |
| 43 | 758 | GQKCVPGHF | 3.000 |
| 44 | 533 | STLRDPNEY | 3.000 |
| 45 | 770 | LAMTYGALF | 3.000 |
| 46 | 384 | EAQTNIAFL | 3.000 |
| 47 | 400 | KSDYADQSL | 3.000 |
| 48 | 650 | LMRLCKNEF | 3.000 |
| 49 | 641 | DAAVGKSSF | 3.000 |
| 50 | 722 | LNIREWVDM | 3.000 |

TABLE XVIII
HLA Peptide Scoring Results-103P3E8-B35-10-mers

| | | | Score (Estimate of Half |
|---|---|---|---|
| | | | Time of Disassociation |
| | Start | Subsequence Residue | of a Molecule Containing |
| Rank | Position | Listing | This Subsequence) |
| 1 | 234 | IPREEQVSTL | 120.000 |
| 2 | 256 | QPYEHVIKNF | 40.000 |
| 3 | 26 | GPNRRRREPL | 20.000 |
| 4 | 532 | LSTLRDPNEY | 15.000 |
| 5 | 116 | RSGRLEREEF | 15.000 |
| 6 | 224 | QARLGDEAKF | 13.500 |
| 7 | 285 | RAQDKAAMQL | 12.000 |
| 8 | 425 | NSLERQIEIL | 10.000 |
| 9 | 443 | DSNDGLRSAL | 10.000 |
| 10 | 574 | DJRDEETFGL | 9.000 |
| 11 | 418 | RAYTEDRNSL | 9.000 |
| 12 | 572 | VPDIRDEETF | 9.000 |
| 13 | 355 | VSQKEDVAAL | 7.500 |
| 14 | 447 | GLRSALENSY | 6.000 |
| 15 | 42 | SPRPTFPQVA | 6.000 |
| 16 | 642 | AAVGKSSFLM | 6.000 |
| 17 | 450 | SALENSYSKF | 6.000 |
| 18 | 456 | YSKFNRSLHI | 6.000 |
| 19 | 329 | RQYETEVGDL | 6.000 |
| 20 | 733 | DAAHETVPIM | 6.000 |
| 21 | 322 | EALSDLRRQY | 6.000 |
| 22 | 523 | LPESCFDSGL | 6.000 |
| 23 | 734 | AAHETVPIML | 6.000 |
| 24 | 663 | SATLGVDFQM | 6.000 |
| 25 | 218 | RAWQDFQARL | 6.000 |
| 26 | 68 | WPRPSREGPA | 6.000 |
| 27 | 37 | HPRRSSPRPT | 6.000 |
| 28 | 272 | QSTEMENLAI | 6.000 |
| 29 | 611 | ISALSPQTDL | 5.000 |
| 30 | 629 | SSQKAYKJVL | 5.000 |
| 31 | 454 | NSYSKFNRSL | 5.000 |
| 32 | 240 | VSTLYQNINL | 5.000 |
| 33 | 214 | ASPGRAWQDF | 5.000 |
| 34 | 605 | SSSRKPISAL | 5.000 |
| 35 | 169 | GGRRRDWGPL | 4.500 |
| 36 | 593 | KPQGSVSEGS | 4.000 |
| 37 | 511 | DPLQRTNCEV | 4.000 |
| 38 | 176 | GPLDPAPAVS | 4.000 |
| 39 | 609 | KPISALSPQT | 4.000 |
| 40 | 86 | RPPPPGGMEA | 4.000 |
| 41 | 566 | DASDTDVPDI | 3.600 |
| 42 | 362 | AALKKQIYDL | 3.000 |
| 43 | 84 | PSRPPPPGGM | 3.000 |
| 44 | 641 | DAAVGKSSFL | 3.000 |
| 45 | 675 | LIVDGERTVL | 3.000 |
| 46 | 14 | GAPHRSRDLL | 3.000 |
| 47 | 644 | VGKSSFLMRL | 3.000 |
| 48 | 270 | RLQSTEMENL | 3.000 |
| 49 | 721 | FLNIREWVDM | 3.000 |
| 50 | 615 | SPQTDLVDDN | 2.000 |

TABLE XIX
Motif-bearing Subseiuences of the 103P3E8 Protein

```
Calculated MW 92.5 kDa, p1 5.35
Protein Motifs
Nuclear protein, with the Nuclear localization sequences being:
      RRRR (5) at 29
      PNRRRRE (5) at 27
      KKQIYDLSMENQKVKKD at 365
Protein Motifs present in 103P3E8:
N-glycosylation site
Number of matches: 5
    1 115-118 NRSG
    2 264-267 NFTR
    3 460-463 NRSL
    4 661-664 NISA
    5 814-S17 NLTG
cAMP- and cGMP- dependent protein kinase phosphorylation site
Number of matches: 3
    1  39-42 RRSS
    2 353-356 KRVS
    3 801-8O4 KKRT
Protein kinase C phosphorylation site
Number of matches: 17
      1 42-44 SPR
      2 117-119 SGR
      3 166-168 SLR
      4 312-314 TRK
      5 341-343 TIK
      6 352-354 SKR
      7 356-358 SQK
      8 630-632 SQK
      9 410-412 TER
    10 478-480 SPK
    11 494-496 SSR
    12 606-608 SSR
    13 534-536 TLR
    14 494-496 SSR
    15 606-608 SSR
    16 607-609 SRK
    17 356-358 SQK
Casein kinase II phosphorylation site
Number of matches: 20
      1 155-158 TFQE
      2 194-197 SEED
      3 274-277 TEME
      4 295-298 SELE
      5 312-315 TRKD
      6 356-359 SQKE
      7 394-397 SELD
      8 410-413 TERD
      9 450-453 SALE
    10 498-501 SYVD
    11 516-519 TNCE
    12 522-525 SLPE
    13 526-529 SCFD
    14 534-537 TLRD
    15 543-546 SEVE
    16 568-571 SDTD
    17 588-591 SVLD
    18 597-600 SVSE
    19 782-785 SAKD
    20 804-807 TDKD
Tyrosine kinase phosphorylation site
449-456 RSALENSY
N-myristoylation site
Number of matches: 9
    1  24-29 GAGPNR
    2  91-96 GGMEAD
    3 165-170 GSLRGG
    4 447-452 GLRSAL
    5 471-476 GNTISR
    6 558-563 GVQESF
    7 564-569 GGDASD
    8 601-606 GSIVSS
    9 775-780 GALFCE
Amidation site
      169-172 GGRR
ATP/GTP-binding site motif A (P-loop)
      640-647 GDAAVGKS
EF-hand calcium-binding domain
Number of matches: 2
    1 113-125 DANRSGRLEREEF
    2 147-159 DADRDGAITFQEF
Leucine zipper pattern
      427-448 LERQIEILQTANRKLHDSNDGL
```

TABLE XX
Frequently Occurring Motifs

| | av. % | | |
|---|---|---|---|
| Name | identity | Description | Potential Function |
| zf-C2H2 | 34% | Zinc finger, C2H2 type | Nucleic acid-binding protein functions as |
| | | | transcription factor, nuclear location |
| | | | probable |
| cytochrome_b_N | 68% | Cytochrome b(N- | membrane bound oxidase, generate |
| | | terminal)/b6/petB | superoxide |
| ig | 19% | Immunoglobulin domain | domains are one hundred amino acids long |
| | | | and include a conserved intradomain |
| | | | disulfide bond. |
| WD40 | 18% | WD domain, G-beta repeat | tandem repeats of about 40 residues, each |
| | | | containing a Trp-Asp motif. Function in |
| | | | signal transduction and protein interaction |
| PDZ | 23% | PDZ domain | may function in targeting signaling |
| | | | molecules to sub-membranous sites |
| LRR | 28% | Leucine Rich Repeat | short sequence motifs involved in protein- |
| | | | protein interactions |
| pkinase | 23% | Protein kinase domain | conserved catalytic core common to both |
| | | | serine/threonine and tyrosine protein |
| | | | kinases containing an ATP binding site and |
| | | | a catalytic site |
| PH | 16% | PH domain | pleckstrin homology involved in |
| | | | intracellular signaling or as constituents of |
| | | | the cytoskeleton |
| EGF | 34% | EGF-like domain | 30-40 amino-acid long found in the |
| | | | extracellular domain of membrane-bound |
| | | | proteins or in secreted proteins |
| rvt | 49% | Reverse transcriptase | |
| | | (RNA-dependent DNA | |
| | | polymerase) | |
| ank | 25% | Ank repeat | Cytoplasmic protein, associates integral |
| | | | membrane proteins to the cytoskeleton |
| oxidored_ql | 32% | NADH- | membrane associated. Involved in proton |
| | | Ubiquinone/plastoquinone | translocation across the membrane |
| | | (complex I), various chains | |
| efhand | 24% | EF hand | calcium-binding domain, consists of a 12 |
| | | | residue loop flanked on both sides by a 12 |
| | | | residue alpha-helical domain |
| rvp | 79% | Retroviral aspartyl protease | Aspartyl or acid proteases, centered on a |
| | | | catalytic aspartyl residue |
| Collagen | 42% | Collagen triple helix repeat | extracellular structural proteins involved in |
| | | (20 copies) | formation of connective tissue. The |
| | | | sequence consists of the G-X-Y and the |
| | | | polypeptide chains forms a triple helix. |
| fn3 | 20% | Fibronectin type III domain | Located in the extracellular ligand-binding |
| | | | region of receptors and is about 200 amino |
| | | | acid residues long with two pairs of |
| | | | cysteines involved in disulfide bonds |
| 7tm_1 | 19% | 7 transmembrane receptor | seven hydrophobic transmembrane regions, |
| | | (rhodopsin family) | with the N-terminus located extracellularly |
| | | | while the C-terminus is cytoplasmic. |
| | | | Signal through G proteins |

1. A method for monitoring 103P3E8 gene products in a biological sample from a patient who has or who is suspected of having cancer, the method comprising:
determining the status of 103P3E8 gene products expressed by cells in a tissue sample from an individual;
comparing the status so determined to the status of 103P3E8 gene products in a corresponding normal sample; and
identifying the presence of aberrant 103P3E8 gene products in the sample relative to the normal sample.

2. A method of monitoring the presence of cancer in an individual comprising:
performing the method of claim 1 whereby the presence of elevated 103P3E8 mRNA or protein expression in the test sample relative to the normal tissue sample provides an indication of the presence or status of a cancer.

3. The method of claim 2, wherein the cancer occurs in a tissue set forth in Table I.

4. A pharmaceutical composition comprising a substance that modulates the status of a cell that expresses 103P3E8.

5. A pharmaceutical composition of claim 4 that comprises an 103P3E8-related protein and a physiologically acceptable carrier.

6. A pharmaceutical composition of claim 4 that comprises an antibody or fragment thereof that specifically binds to a 103P3E8-related protein and a physiologically acceptable carrier.

7. A pharmaceutical composition of claim 4 that comprises a polynucleotide that encodes a single chain monoclonal antibody that immunospecifically binds to an 103P3E8-related protein and a physiologically acceptable carrier.

8. A pharmaceutical composition of claim 4 that comprises a polynucleotide comprising a 103P3E8-related protein coding sequence and a physiologically acceptable carrier.

9. A pharmaceutical composition of claim 4 that comprises an antisense polynucleotide complementary to a polynucleotide having a 103P3E8 coding sequence and a physiologically acceptable carrier.

10. A pharmaceutical composition of claim 4 that comprises a ribozyme capable of cleaving a polynucleotide having 103P3E8 coding sequence and a physiologically acceptable carrier.

11. A method of treating a patient with a cancer that expresses 103P3E8, the method comprising steps of:
administering to said patient a vector that comprises the composition of claim 7, such that the vector delivers the single chain monoclonal antibody coding sequence to the cancer cells and the encoded single chain antibody is expressed intracellularly therein.

12. A method of inhibiting in a patient the development of a cancer that expresses 103P3E8, the method comprising:
administering to the patient an effective amount of the composition of claim 4.

13. A method of generating a mammalian immune response directed to 103P3E8, the method comprising:
exposing the mammal's immune system to an immunogenic portion of an 103P3E8-related protein or a nucleotide sequence that encodes said protein, whereby an immune response is generated to 103P3E8.

14. A method of delivering a cytotoxic agent to a cell that expresses 103P3E8, said method comprising:
conjugating the cytotoxic agent to an antibody or fragment thereof that specifically binds to a 103P3E8 epitope; and,
exposing the cell to the antibody-agent conjugate.

15. A method of inducing an immune response to a 103P3E8 protein, said method comprising:
providing a 103P3E8-related protein that comprises at least one T cell or at least one B cell epitope;
contacting the epitope with an immune system T cell or B cell respectively, whereby the immune system T cell or B cell is induced.

16. The method of claim 15, wherein the immune system cell is a B cell, whereby the induced B cell generates antibodies that specifically bind to the 103P3E8-related protein.

17. The method of claim 15, wherein the immune system cell is a T cell that is a cytotoxic T cell (CTL), whereby the activated CTL kills an autologous cell that expresses the 103P3E8 protein.

18. The method of claim 15, wherein the immune system cell is a T cell that is a helper T cell (HTL), whereby the activated HTL secretes cytokines that facilitate the cytotoxic activity of a CTL or the antibody producing activity of a B cell.

19. An antibody or fragment thereof that specifically binds to a 103P3E8-related protein.

20. The antibody or fragment thereof of claim 19, which is monoclonal.

21. A recombinant protein comprising the antigen-binding region of a monoclonal antibody of claim 20.

22. The antibody or fragment thereof of claim 19, which is labeled with a detectable marker.

23. The recombinant protein of claim 21, which is labeled with a detectable marker.

24. The antibody fragment of claim 19, which is an Fab, F(ab′)2, Fv or sFv fragment.

25. The antibody of claim 19, which is a human antibody.

26. The recombinant protein of claim 21, which comprises murine antigen binding region residues and human constant region residues.

27. A non-human transgenic animal that produces an antibody of claim 19.

28. A hybridoma that produces an antibody of claim 20.

29. A single chain monoclonal antibody that comprises the variable domains of the heavy and light chains of a monoclonal antibody of claim 20.

30. A vector comprising a polynucleotide that encodes a single chain monoclonal antibody of claim 29 that immunospecifically binds to a 103P3E8-related protein.

31. An assay for detecting the presence of a 103P3E8-related protein or polynucleotide in a biological sample from a patient who has or who is suspected of having cancer, comprising steps of:
contacting the sample with an antibody or another polynucleotide, respectively, that specifically binds to the 103P3E8-related protein or polynucleotide, respectively; and,
detecting the binding of 103P3E8-related protein or polynucleotide, respectively, in the sample thereto.

32. An assay of claim 31 for detecting the presence of a 103P3E8-related protein or polynucleotide in a biological sample from a patient who has or who is suspected of having cancer, comprising the steps of:
obtaining a sample from a patient who has or who is suspected of having cancer, evaluating said sample in the presence of a 103P3E8-related protein or polynucleotide, whereby said evaluating step produces a result that indicates the presence or amount of 103P3E8-related protein or polynucleotide, respectively.

33. An assay of claim 31 for detecting the presence of an 103P3E8 polynucleotide in a biological sample, comprising:
(a) contacting the sample with a polynucleotide probe that specifically hybridizes to a polynucleotide encoding an 103P3E8-related protein having an amino acid sequence shown in FIG. 2 or FIG. 4; and
(b) detecting the presence of a hybridization complex formed by the hybridization of the probe with 103P3E8 polynucleotide in the sample, wherein the presence of the hybridization complex indicates the presence of 103P3E8 polynucleotide within the sample.

34. An assay for detecting the presence of 103P3E8 mRNA in a biological sample from a patient who has or who is suspected of having cancer, said method comprising:
(a) producing cDNA from the sample by reverse transcription using at least one primer;
(b) amplifying the cDNA so produced using 103P3E8 polynucleotides as sense and antisense primers to amplify 103P3E8 cDNAs therein, wherein the 103P3E8 polynucleotides used as the sense and antisense probes are capable of amplifying the 103P3E8 cDNA contained within the plasmid as deposited with American Type Culture Collection as Accession No. PTA-1262;
(c) detecting the presence of the amplified 103P3E8 cDNA.
